(19) United States
(12) Patent Application Publication
(10) Pub. No.: US 2026/0266839 A1
(43) Pub. Date: Sep. 10, 2026

(54) METHODS AND COMPOSITIONS FOR DIAGNOSIS OF ECTOPIC PREGNANCY, INTRAUTERINE PREGNANCY, AND SPONTANEOUS ABORTION

(71) Applicants: The Trustees of the University of Pennsylvania, Philadelphia, PA (US); The Wistar Institute of Anatomy and Biology, Philadelphia, PA (US)

(72) Inventors: Kurt T. Barnhart, Bryn Mawr, PA (US); David W. Speicher, Berwyn, PA (US)

(21) Appl. No.: 19/166,374

(22) PCT Filed: Mar. 18, 2024
(86) PCT No.: PCT/US2024/020415

§ 371 Date: Sep. 17, 2025

Related U.S. Application Data
(60) Provisional application No. 63/491,011, filed on Mar. 17, 2023.

(51) Int. Cl. G01N 33/68; C12Q 1/6883

(52) U.S. Cl. CPC G01N 33/689; C12Q 1/6883; C12Q 2600/158; G01N 2800/368

(57) ABSTRACT

Methods and compositions are provided for diagnosing ectopic pregnancy or nonviable pregnancy in a mammalian subject by detecting changes in expression of the selected genes, gene fragments or transcripts or expression products, or changes in the expression levels of one or more of proteins or peptide fragments.

### CROSS-REFERENCE TO RELATED APPLICATIONS

This application claims priority of U.S. Provisional application No. 63/491,011 filed Mar. 17, 2023, the entire contents being incorporated herein by reference as though set forth in full.

### STATEMENT REGARDING FEDERALLY SPONSORED RESEARCH OR DEVELOPMENT

This invention was made with government support under grant numbers HD076279 and HD110448 awarded by the National Institutes of Health. The government has certain rights in the invention.

### BACKGROUND

In a field of consistently improving techniques and technologies, the distinction between a normal versus abnormal early pregnancy remains a vexing clinical challenge for health care providers and their female patients. The importance of being able to accurately make the diagnosis of either a viable intrauterine pregnancy (IUP), pregnancy loss (SAB), or ectopic pregnancy (EP) cannot be overstated, with each of these outcomes necessitating unique treatment pathways as well as contributing strikingly different rates of maternal morbidity and mortality.

The ability to detect pathology at an early stage that facilitates noninvasive intervention, reduces the chance of severe morbidity, and avoids potentially detrimental intervention from misdiagnosis, is a goal for all clinicians. The initial triage of women presenting with early pregnancy currently includes the diagnostic integration of patient symptoms, laboratory serum human chorionic gonadotropin (hCG) levels, and pelvic ultrasound with no single entity being diagnostic for any specific pregnancy outcome. Despite improving technical skill and technology, ultrasound at first evaluation of early pregnancy may be inconclusive in up to 40% of women, particularly in those with pregnancies less than seven weeks gestation and hCG values below the discriminatory zone.

In reproductive medicine, biomarker development has become a rapidly evolving field channeled towards conditions that affect both acute and long-term maternal health. In conditions with the potential for the highest levels of morbidity and mortality such as ectopic pregnancy (EP), biomarkers can change the way we diagnosis and manage women with a possible abnormal early pregnancy.

For more than three decades, various biomarkers have been proposed in individual studies, but only a fraction of these studies have taken the subsequent steps of biomarker assay development that ultimately led to integration into clinical practice. It has been proposed to predict both viability (intrauterine pregnancy [IUP] vs. EP and spontaneous abortion [SAB]) and location (EP vs. IUP and SAB). Proposed models using putative markers have not achieved sufficiently high test characteristics for clinical utility. In this study, we sought to increase the pool of biomarkers by assessing novel biomarkers with a combination of identification of new markers based on biological plausibility and markers identified with agnostic discovery. The use of new markers can further improve accuracy and discrimination ultimately leading to widespread clinical use.

What is needed is improved compositions and methods for diagnosing ectopic and non-viable pregnancies.

### SUMMARY OF THE INVENTION

In one aspect, the invention includes diagnostic reagents or kits for use in diagnosing an ectopic pregnancy or a non-viable pregnancy in a mammalian subject comprising (a) a ligand that that binds to a peptide or protein selected from the biomarkers identified in Tables 2A and 2B, or (b) a combination of ligand (a), wherein each ligand binds to a different peptide or protein; wherein at least one of the ligands is associated with a detectable label or with a substrate. In certain embodiments, the peptide or proteins are selected from the group consisting of: i. disintegrin and metalloproteinase domain-containing protein 12 (ADAM12), ii. Glycoprotein hormones alpha chain (CGA), iii. beta human chorionic gonadotropin (hCG), iv. Pappalysin-1 (PAPPA), v. pregnancy-specific beta-1-glycoprotein 1 (PSG1), vi. pregnancy-specific beta-1-1glycoprotein 3 (PSG3), vii. pregnancy-specific beta-1-glycoprotein 9 (PSG9), viii. soluble fms-like tyrosine kinase-1 (sFLT), ix. growth-differentiation faction-15 (GDF15), x. progesterone (PRG), and xi. tissue factor pathway inhibitor 2 (TFPI2), or xii. a combination of ligands, each binding a peptide or protein of (i) through (xi).

In certain embodiments, the reagent or kit diagnoses pregnancy viability and the peptide or protein is PSG3. In certain embodiments, the reagent or kit diagnoses pregnancy viability and the peptides or proteins are PAPPA and PSG3. In certain embodiments, the reagent or kit diagnoses pregnancy viability and the peptides or proteins are CGA and PAPPA. In certain embodiments, the reagent or kit diagnoses pregnancy viability and the peptides or proteins are CGA and PSG3. In certain embodiments, the reagent or kit diagnoses pregnancy viability and the peptides or proteins are CGA, PAPPA, and PSG3. In certain embodiments, the reagent or kit diagnoses pregnancy viability and the peptides or proteins are PSG3, PSG9, CGA, hCG, PRG, and GF15. In certain embodiments, the reagent or kit diagnoses pregnancy viability and the peptides or proteins are PSG3 and PSG9. In certain embodiments, the reagent or kit diagnoses pregnancy viability and the peptides or proteins are ADAM12, CGA, hCG, PAPPA, PSG1, PSG3, PSG9, sFLT, GDF15, and PRG. In certain embodiments, the reagent or kit diagnoses pregnancy viability and the peptides or proteins are all of biomarkers (i)-(xi).

In certain embodiments, the reagent or kit diagnoses pregnancy location and the peptides or proteins are PSG3 and TFPI2. In certain embodiments, the reagent or kit diagnoses pregnancy location and the peptide or protein is sFLT. In certain embodiments, the reagent or kit diagnoses pregnancy location and the peptide or protein is PSG1. In certain embodiments, the reagent or kit diagnoses pregnancy location and the peptides or proteins are sFLT, PSG3, and TFPI2. In certain embodiments, the reagent or kit diagnoses pregnancy location and the peptides or proteins are PSG1, PSG3, and TFPI2. In certain embodiments, the reagent or kit diagnoses pregnancy location and the peptides or proteins are ADAM12, CGA, hCG, PAPPA, PSG1, PSG3, PSG9, sFLT, GDF15, and TFPI2. In certain embodiments, the reagent or kit diagnoses pregnancy location and the peptides or proteins are all of biomarkers (i)-(xi).

In another aspect, the invention includes diagnostic reagent or kit for use in diagnosing an ectopic pregnancy in a mammalian subject comprising: (a) a ligand that that binds to a peptide or protein selected from the group consisting of: i. pregnancy specific beta-1-glycoprotein 9 (PSG9), ii. pregnancy specific beta-1 glycoprotein 1 (PSG1), iii. insulin-like growth factor binding protein 1 (IGFBP1), iv. kisspeptin (KISS1), v. pregnancy specific beta-1 glycoprotein 3 (PSG3), and vi. beta-parvin (PARVB); or (b) a combination of ligand (a), wherein each ligand binds to a different peptide or protein (i) through (vi); wherein at least one of the ligands is associated with a detectable label or with a substrate. In certain embodiments, the reagents or kits further include a ligand that binds to a protein or peptide fragment selected from the group consisting of: vii. EH domain-containing protein 3 (EHD3); viii. WAP four-disulfide core domain protein 2 (HE4); and ix. quiescin sulfhydryl oxidase 2 (QSOX2); or (d) a combination of ligands, each ligand binding a different peptide or protein of (vii) through (ix).

In yet another aspect, the invention includes diagnostic reagents or kits for use in diagnosing an ectopic pregnancy in a mammalian subject comprising: (a) a polynucleotide or oligonucleotide sequence, which hybridizes to a gene, gene fragment, gene transcript or expression product selected from the group consisting of: i. pregnancy specific beta-1-glycoprotein 9 (PSG9), ii. pregnancy specific beta-1 glycoprotein 1 (PSG1), iii. insulin-like growth factor binding protein 1 (IGFBP1), iv. kisspeptin (KISS1), v. pregnancy specific beta-1 glycoprotein 3 (PSG3), and vi. beta-parvin (PARVB); or (b) a combination of ligands (a) (i) through (a) (vi) wherein at least one polynucleotide or oligonucleotide sequence is associated with a detectable label or with a substrate. In certain embodiments, the diagnostic reagents or kits further comprise a polynucleotide or oligonucleotide sequence, which hybridizes to a gene, gene fragment, gene transcript or expression product of a biomarker selected from the group consisting of: vii. EH domain-containing protein 3 (EHD3); viii. WAP four-disulfide core domain protein 2 (HE4); and ix. quiescin sulfhydryl oxidase 2 (QSOX2); or (d) a combination of sequences, each sequence hybridizing to a different biomarker of (vii) through (ix).

In another aspect, the invention includes diagnostic reagent or kit for use in diagnosing a nonviable pregnancy in a mammalian subject comprising: (a) a ligand that that binds to a peptide or protein selected from the group consisting of: i. pregnancy specific beta-1-glycoprotein 9 (PSG9), ii. pregnancy specific beta-1 glycoprotein 3 (PSG3), iii. EH domain-containing protein 3 (EHD3), iv. kisspeptin (KISS1), v. WAP four-disulfide core domain protein 2 (HE4), vi. quiescin sulfhydryl oxidase 2 (QSOX2), and vii. pregnancy specific beta-1 glycoprotein 1 (PSG1); or (b) a combination of ligand (a), wherein each ligand binds to a different peptide or protein (i) through (vii); wherein at least one of the ligands is associated with a detectable label or with a substrate. In certain embodiments, the reagents or kits further include a ligand that binds to a protein or peptide fragment selected from the group consisting of: viii. vii. insulin-like growth factor binding protein 1 (IGFBP1); and ix. beta-parvin (PARVB); or (d) a combination of ligands, each ligand binding a different peptide or protein of (viii) through (ix).

In another aspect, the invention includes diagnostic reagent or kit for use in diagnosing a nonviable pregnancy in a mammalian subject comprising: (a) a polynucleotide or oligonucleotide sequence, which hybridizes to a gene, gene fragment, gene transcript or expression product of a biomarker selected from the group consisting of: i. pregnancy specific beta-1-glycoprotein 9 (PSG9), ii. pregnancy specific beta-1 glycoprotein 3 (PSG3), iii. EH domain-containing protein 3 (EHD3), iv. kisspeptin (KISS1), v. WAP four-disulfide core domain protein 2 (HE4), vi. quiescin sulfhydryl oxidase 2 (QSOX2), and vii. pregnancy specific beta-1 glycoprotein 1 (PSG1); or (b) a combination of ligand (a), wherein each ligand binds to a different peptide or protein (i) through (vii); wherein at least one of the polynucleotide or oligonucleotide sequence is associated with a detectable label or with a substrate. In certain embodiments, the reagents or kits further include a polynucleotide or oligonucleotide sequence, which hybridizes to a gene, gene fragment, gene transcript or expression product of a biomarker selected from the group consisting of: viii. insulin-like growth factor binding protein 1 (IGFBP1); and ix. beta-parvin (PARVB); or (d) a combination of sequences, each sequence hybridizing to a different biomarker of (viii) through (ix).

In certain embodiments, the above reagents and kits comprises a substrate upon which said polynucleotide or oligonucleotide or ligand is immobilized. In another embodiment said ligands or said expression products are proteins or peptides. In certain embodiments, the ligand is an antibody or fragment thereof. In certain embodiments, the polynucleotide or oligonucleotide sequence or ligand is associated with a detectable label. In certain embodiments, the reagents or kits further comprise a microarray, a microfluidics card, a chip or a chamber. In certain embodiments, said polynucleotide or oligonucleotide is part of a primer-probe set, and said kit comprises both primer and probe, wherein each said primer-probe set amplifies a different gene, gene fragment or gene expression product.

In one aspect, the invention includes methods for diagnosing an ectopic pregnancy or a non-viable pregnancy in a mammalian subject comprising (a) measuring in a biological fluid sample of the subject the expression level of a gene, gene fragment, gene transcript or expression product selected from the biomarkers identified in Tables 2A and 2B, comparing said subject's selected gene, gene fragment, gene transcript or expression product expression level with the level of the same gene, gene fragment, gene transcript or expression product in the biological fluid of a reference or control female mammalian subject having a normal intrauterine pregnancy (IUP), wherein changes in expression of the subject's selected gene, gene fragment, gene transcript or expression products from those of the reference or control correlates with a diagnosis of ectopic pregnancy or non-viable pregnancy. In certain embodiments, the biomarkers are selected from the group consisting of: i. disintegrin and metalloproteinase domain-containing protein 12 (ADAM12), ii. Glycoprotein hormones alpha chain (CGA), iii. beta human chorionic gonadotropin (hCG), iv. Pappalysin-1 (PAPPA), v. pregnancy-specific beta-1-glycoprotein 1 (PSG1), vi. pregnancy-specific beta-1-1glycoprotein 3 (PSG3), vii. pregnancy-specific beta-1-glycoprotein 9 (PSG9), viii. soluble fms-like tyrosine kinase-1 (sFLT), ix. growth-differentiation faction-15 (GDF15), x. progesterone (PRG), and xi. tissue factor pathway inhibitor 2 (TFPI2), or xii. a combination of ligands, each binding a peptide or protein of (i) through (xi).

In certain embodiments, the method diagnoses pregnancy viability and the peptide or protein is PSG3. In certain embodiments, the method diagnoses pregnancy viability and the peptides or proteins are PAPPA and PSG3. In certain embodiments, the method diagnoses pregnancy viability and the peptides or proteins are CGA and PAPPA. In certain embodiments, the method diagnoses pregnancy viability and the peptides or proteins are CGA and PSG3. In certain embodiments, the method diagnoses pregnancy viability and the peptides or proteins are CGA, PAPPA, and PSG3. In certain embodiments, the method diagnoses pregnancy viability and the peptides or proteins are PSG3, PSG9, CGA, hCG, PRG, and GF15. In certain embodiments, the method diagnoses pregnancy viability and the peptides or proteins are PSG3 and PSG9. In certain embodiments, the method diagnoses pregnancy viability and the peptides or proteins are ADAM12, CGA, hCG, PAPPA, PSG1, PSG3, PSG9, sFLT, GDF15, and PRG. In certain embodiments, the method diagnoses pregnancy viability and the peptides or proteins are all of biomarkers (i)-(xi).

In certain embodiments, the method diagnoses pregnancy location and the peptides or proteins are PSG3 and TFPI2. In certain embodiments, the method diagnoses pregnancy location and the peptide or protein is sFLT. In certain embodiments, the method diagnoses pregnancy location and the peptide or protein is PSG1. In certain embodiments, the method diagnoses pregnancy location and the peptides or proteins are sFLT, PSG3, and TFPI2. In certain embodiments, the method diagnoses pregnancy location and the peptides or proteins are PSG1, PSG3, and TFPI2. In certain embodiments, the method diagnoses pregnancy location and the peptides or proteins are ADAM12, CGA, hCG, PAPPA, PSG1, PSG3, PSG9, sFLT, GDF15, and TFPI2. In certain embodiments, the method diagnoses pregnancy location and the peptides or proteins are all of biomarkers (i)-(xi).

In another aspect, the invention includes methods for diagnosing an ectopic pregnancy in a mammalian subject comprising: (a) measuring in a biological fluid sample of the subject the expression level of a gene, gene fragment, gene transcript or expression product selected from the group consisting of: i. pregnancy specific beta-1-glycoprotein 9 (PSG9), ii. pregnancy specific beta-1 glycoprotein 1 (PSG1), iii. insulin-like growth factor binding protein 1 (IGFBP1), iv. kisspeptin (KISS1), v. pregnancy specific beta-1 glycoprotein 3 (PSG3), and vi. beta-parvin (PARVB); and (b) comparing said subject's selected gene, gene fragment, gene transcript or expression product expression level with the level of the same gene, gene fragment, gene transcript or expression product in the biological fluid of a reference or control female mammalian subject having a normal intrauterine pregnancy (IUP), wherein changes in expression of the subject's selected gene, gene fragment, gene transcript or expression products from those of the reference or control correlates with a diagnosis of ectopic pregnancy. In certain embodiments, the methods further comprise measuring in said biological fluid sample of the subject the expression level of a gene, gene fragment, gene transcript or expression product selected from the group consisting of: vii. EH domain-containing protein 3 (EHD3); viii. WAP four-disulfide core domain protein 2 (HE4); and ix. quiescin sulfhydryl oxidase 2 (QSOX2); or (d) a combination of ligands, each ligand binding a different peptide or protein of (vii) through (ix).

In another aspect, the invention includes methods for diagnosing a nonviable pregnancy in a mammalian subject comprising: (a) measuring in a biological fluid sample of the subject the expression level of a gene, gene fragment, gene transcript or expression product selected from the group consisting of: i. pregnancy specific beta-1-glycoprotein 9 (PSG9), ii. pregnancy specific beta-1 glycoprotein 3 (PSG3), iii. EH domain-containing protein 3 (EHD3), iv. kisspeptin (KISS1), v. WAP four-disulfide core domain protein 2 (HE4), vi. quiescin sulfhydryl oxidase 2 (QSOX2), and vii. pregnancy specific beta-1 glycoprotein 1 (PSG1); and (b) comparing said subject's selected gene, gene fragment, gene transcript or expression product expression level with the level of the same gene, gene fragment, gene transcript or expression product in the biological fluid of a reference or control female mammalian subject having a normal intrauterine pregnancy (IUP), wherein changes in expression of the subject's selected gene, gene fragment, gene transcript or expression product s from those of the reference or control correlates with a diagnosis of ectopic pregnancy. In certain embodiments, the methods further comprise measuring in said biological fluid sample of the subject the expression level of a gene, gene fragment, gene transcript or expression product selected from the group consisting of: viii. insulin-like growth factor binding protein 1 (IGFBP1); and ix. beta-parvin (PARVB); or (d) a combination of sequences, each sequence hybridizing to a different biomarker of (viii) through (ix).

In certain embodiments, said change in expression level of each said selected gene, gene fragment, gene transcript or expression product comprises an upregulation in comparison to said reference or control or a downregulation in comparison to said reference or control.

In another aspect, the invention includes methods for diagnosing an ectopic pregnancy in a mammalian subject comprising: (a) measuring in a biological fluid sample of the subject the expression level of a protein or peptide fragment thereof selected from the group consisting of: i. pregnancy specific beta-1-glycoprotein 9 (PSG9), ii. pregnancy specific beta-1 glycoprotein 1 (PSG1), iii. insulin-like growth factor binding protein 1 (IGFBP1), iv. kisspeptin (KISS1), v. pregnancy specific beta-1 glycoprotein 3 (PSG3), and vi. beta-parvin (PARVB); and (b) comparing said subject's expression level with the selected protein or peptide fragment with the level of the same protein or peptide in the biological fluid of a reference or control female mammalian subject having a normal intrauterine pregnancy (IUP), wherein changes in expression of the subject's selected protein or peptide fragment from those of the reference or control correlates with a diagnosis of ectopic pregnancy. In certain embodiments, the methods further comprise measuring in said biological fluid sample of the subject the expression level of a protein or peptide fragment thereof selected from the group consisting of: vii. EH domain-containing protein 3 (EHD3); viii. WAP four-disulfide core domain protein 2 (HE4); and ix. quiescin sulfhydryl oxidase 2 (QSOX2); or (d) a combination of ligands, each ligand binding a different peptide or protein of (vii) through (ix).

In another aspect, the invention includes methods for diagnosing a nonviable pregnancy in a mammalian subject comprising: (a) measuring in a biological fluid sample of the subject the expression level of a protein or peptide fragment thereof selected from the group consisting of: i. pregnancy specific beta-1-glycoprotein 9 (PSG9), ii. pregnancy specific beta-1 glycoprotein 3 (PSG3), iii. EH domain-containing protein 3 (EHD3), iv. kisspeptin (KISS1), v. WAP four-disulfide core domain protein 2 (HE4), vi. quiescin sulfhydryl oxidase 2 (QSOX2), and vii. pregnancy specific beta-1 glycoprotein 1 (PSG1); and (b) comparing said subject's expression level with the selected protein or peptide fragment with the level of the same protein or peptide in the biological fluid of a reference or control female mammalian subject having a normal intrauterine pregnancy (IUP), wherein changes in expression of the subject's selected protein or peptide fragment from those of the reference or control correlates with a diagnosis of nonviable pregnancy. In certain embodiments, the methods further comprise measuring in said biological fluid sample of the subject the expression level of a protein or peptide fragment thereof selected from the group consisting of: viii. insulin-like growth factor binding protein 1 (IGFBP1); and ix. beta-parvin (PARVB); or (d) a combination of sequences, each sequence hybridizing to a different biomarker of (viii) through (ix).

In certain embodiments, the change in expression level of each said selected protein or peptide fragment comprises an upregulation in comparison to said reference or control or a downregulation in comparison to said reference or control.

In certain embodiments, these methods further comprise measuring the expression level of a protein or peptide fragment, or a polynucleotide or oligonucleotide sequence, which hybridizes to a gene, gene fragment, gene transcript or expression product selected from Tables 2A and 2B. In certain embodiments, the method further comprises terminating the pregnancy.

In yet another aspect, the invention includes methods of diagnosing pregnancy location in a pregnant subject, the method comprising a) obtaining a measurement of Activin A in a sample from the subject, and b) obtaining a measurement of glycodelin in a sample from the subject, and c) diagnosing the pregnancy as ectopic when the sample contains less than or equal to 275.1 pg/mL Activin A, and/or the sample contains from 0.17 ng/ml to 1.39 ng/ml glycodelin. In certain embodiments, the method further comprises diagnosing the pregnancy as intrauterine when the sample contains less than or equal to 0.17 ng/ml or greater than 1.39 ng/ml glycodelin.

In one aspect, provided is a method of diagnosing pregnancy location in a pregnant subject, the method comprising a) obtaining a measurement of Activin A in a sample from the subject, b) obtaining a measurement of ADAM 12 in a sample from the subject, and c) diagnosing the pregnancy as ectopic when the sample contains less than or equal to 179.45 pg/ml Activin A and the sample contains less than or equal to 0.42 ng/ml or greater than 0.89 ng/mL ADAM 12. In certain embodiments, the method further comprises diagnosing the pregnancy is intrauterine when the sample contains greater than 179.45 pg/mL Activin A or the sample contains from 0.42 ng/ml to 0.89 ng/mL ADAM12.

In yet another aspect, the invention includes methods of diagnosing pregnancy viability in a pregnant subject, the method comprising a) obtaining a measurement of progesterone in a sample from the subject, b) diagnosing the pregnancy as nonviable when the sample contains greater than or equal to 7.99 ng/ml progesterone. In certain embodiments, the method further comprises diagnosing the pregnancy as intrauterine when the sample contains less than 7.99 ng/mL.

In another aspect, the invention includes methods of diagnosing pregnancy viability in a pregnant subject, the method comprising a) obtaining a measurement of progesterone in a sample from the subject, b) obtaining a measurement of PAPPA in a sample from the subject, and c) diagnosing the pregnancy as nonviable when the sample contains less than or equal to 17.28 ng/ml progesterone or the sample contains less than or equal to 0.22 ng/mL PAPPA and greater than 17.84 ng/mL ADAM 12. In certain embodiments, the method further comprises diagnosing the pregnancy as intrauterine when the sample contains greater than 17.28 pg/mL progesterone and greater than 0.22 ng/mL of PAPPA or the sample contains between 17.28 pg/mL and 17.84 ng/mL progesterone and less than or equal to 0.22 ng/ml PAPPA.

In one aspect, the invention includes methods of diagnosing pregnancy location in a pregnant subject, the method comprising a) obtaining a measurement of sFLT in a sample from the subject, b) diagnosing the pregnancy as ectopic when the sample contains less than 598.0 pg/ml sFLT. In another aspect, the invention includes methods of diagnosing pregnancy location in a pregnant subject, the method comprising a) obtaining a measurement of PSG1 in a sample from the subject, b) diagnosing the pregnancy as ectopic when the sample contains less than 22.5 ng/ml progesterone. In another aspect, the invention includes methods diagnosing pregnancy location in a pregnant subject, the method comprising a) obtaining a measurement of PSG3 in a sample from the subject, b) obtaining a measurement of TFPI2 in a sample from the subject, and c) diagnosing the pregnancy as ectopic when the sample contains less than 34.16 ng/ml PSG3 and/or the sample contains less than 282.5 pg/mL TFPI2. In another aspect, the invention includes methods diagnosing pregnancy location in a pregnant subject, the method comprising a) obtaining a measurement of sFLT in a sample from the subject, b) obtaining a measurement of PSG3 in a sample from the subject, c) obtaining a measurement of TFPI2 in a sample from the subject, and d) diagnosing the pregnancy as ectopic when the sample contains less than 598.0 pg/mL sFLT, less than 34.16 ng/ml PSG3, and/or less than 282.5 pg/mL TFPI2. In another aspect, the invention includes methods diagnosing pregnancy viability in a pregnant subject, the method comprising a) obtaining a measurement of PSG3 in a sample from the subject, b) diagnosing the pregnancy as nonviable when the sample contains less than 16.7 ng/mL PSG3. In another aspect, the invention includes methods diagnosing pregnancy viability in a pregnant subject, the method comprising a) obtaining a measurement of PAPA in a sample from the subject, b) obtaining a measurement of PSG3 in a sample from the subject, and c) diagnosing the pregnancy as nonviable when the sample contains less than 25.57 ng/ml PSG3 or greater than 0. 29.57 ng/mL PSG3 and greater than 0.52 ng/ml PAPPA. In another aspect, the invention includes methods diagnosing pregnancy viability in a pregnant subject, the method comprising a) obtaining a measurement of CGA in a sample from the subject, b) obtaining a measurement of PAPPA in a sample from the subject, and c) diagnosing the pregnancy as nonviable when the sample contains less than 10960 mIU/mL CGA or greater than 10960 mIU/mL CGA in a sample and greater than 0.585 ng/ml PAPPA. In another aspect, the invention includes methods diagnosing pregnancy viability in a pregnant subject, the method comprising a) obtaining a measurement of PSG3 in a sample from the subject, b) obtaining a measurement of PAPPA in a sample from the subject, c) obtaining a measurement of CGA in a sample from the subject, and d) diagnosing the pregnancy as nonviable when the sample contains less than less than 16.7 pg/mL PSG3 and less than 10960 mIU/ml CGA or greater than 10960 mIU/mL CGA and greater than 0.585 ng/ml PAPPA in a sample.

In certain embodiments, the above methods further comprise terminating the pregnancy when the pregnancy is diagnosed as ectopic or nonviable.

Other aspects and advantages of the invention will be readily apparent from the following detailed description of the invention.

### DESCRIPTION OF THE FIGURES

FIG. 1A-1B: Derived decision trees to predict Pregnancy Location (FIG. 1A) and derived decision trees to predict Pregnancy Viability (FIG. 1B) are provided. Final diagnosis of each individual was only made if classification of the subject was in agreement from both trees. If both trees classified the subject as a case or a control, definitive classification was determined. If the classification did not agree (i.e., one tree classified the subject as case and the other a control) the subjects who were not definitively diagnosed and were labeled 10 ‘indeterminate”.

FIG. 2: Area Under the Curve (AUC) with 95% Confidence Intervals for an embodiment of the invention for Location or Viability.

FIG. 3: Combined Area Under the Curve (AUC) with 95% CI for an embodiment of the invention for Location and/or Viability.

FIG. 4: A graphical depiction of the development of the Random Forest model.

FIG. 5A-5B: Comparison models for pregnancy viability (FIG. 5A) and location (FIG. 5B).

### DETAILED DESCRIPTION OF THE INVENTION

Provided herein are diagnostic compositions, and methods of using the same, to help identify women with an ectopic pregnancy based on a panel of multiple markers used simultaneously. In addition, provided herein is a clinical test for location of pregnancy (i.e., prediction of the presence or absence of an ectopic pregnancy). Use of these compositions and methods will change clinical care, enhancing early diagnosis and preventing complication inherent to ectopic pregnancy including death, internal bleeding, urgent need for surgery, loss of fertility and loss of a fallopian tube. Early diagnosis also would allow non-invasive medical intervention of an identified ectopic pregnancy. A woman can be spared unnecessary surgical intervention (uterine evacuation or laparoscopy often needed for diagnosis or treatment) and offered medical management or personalized care to treatment an ectopic pregnancy. If an intrauterine pregnancy (non-ectopic pregnancy) is predicted accurately, a woman can be offered reassurance of an ongoing pregnancy and safely triaged for obstetrical care. Either of these strategies could dramatically decrease the frequency of outpatient surveillance and use of invasive and costly diagnostic tests and procedures.

It is to be noted that the term “a” or “an” as used herein refers to one or more. As such, the terms “a” (or “an”), “one or more,” and “at least one” are used interchangeably herein.

As used herein, the term “about” means a variability of 10% from the reference given, unless otherwise specified.

“Patient” or “subject” as used herein means a female mammalian animal, including a human, a veterinary or farm animal, a domestic animal or pet, and animals normally used for clinical research. In one embodiment, the subject of these methods and compositions is a human.

“Control” or “Control subject” as used herein refers to both an individual female with IUP or the pooled biological fluids (e.g., sera) from multiple females with IUP or numerical or graphical averages of the expression levels of the selected biomarkers obtained from large groups of females with IUP. Such controls are the types that are commonly used in similar diagnostic assays for other biomarkers. Selection of the particular class of controls depends upon the use to which the diagnostic methods and compositions are to be put by the physician. As used herein, the term “predetermined control” refers to a numerical level, average, mean or average range of the expression of a biomarker in a defined population. The predetermined control level is preferably provided by using the same assay technique as is used for measurement of the subject's biomarker levels, to avoid any error in standardization. For example, the control may comprise a single healthy pregnant mammalian subject at the same time of pregnancy as the subject. In another embodiment, the control comprises a population of multiple healthy pregnant mammalian subjects at the same time of pregnancy as the subject or multiple healthy IUP mammalian subjects. In another embodiment, the control comprises the same subject at an earlier time in the pregnancy. In addition, a predetermined control may also be a negative predetermined control. In one embodiment, a negative predetermined control comprises one or multiple subjects who have EP or SAB. The control can refer to a numerical average, mean or average range of the expression of one or more biomarkers, in a defined population, rather than a single subject.

The phrase “intrauterine pregnancy” or “IUP” or “viable pregnancy” refers to a pregnancy that results in a live birth or confirmed second trimester viability.

The phrase “ectopic pregnancy” or “EP” refers to a pregnancy in which the fetus develops outside of the uterus. Typically, implantation occurs in the fallopian tube. Ectopic pregnancy generally requires surgical removal of the fetus, or treatment with methotrexate within 48 hours of diagnosis. Ectopic pregnancy can be identified by ultrasound identification of a gestational sac with a yolk sac or crown rump in adnexa, lack of products of conception or chorionic villi after dilation and curettage in setting of abnormal hCG rise, or Surgical identification of an ectopic pregnancy within 48 hours of serum collection for EP.

The phrase “non-viable pregnancy”, “pregnancy loss”, “spontaneous abortion”, and “SAB” refers to an intrauterine pregnancy that cannot possibly result in a liveborn baby. Non-viable pregnancy includes fetal demise, where crown rump length [CRL]≥0.7 cm without fetal cardiac activity; or ultrasound with no fetal cardiac activity after a previous ultrasound showed fetal cardiac activity within 48 hours of serum collection, or the is no fetal cardiac activity within the first 7 weeks of pregnancy, and anembryonic gestation, where no fetus is present in the gestational sac; a gestational sac ≥1.6 cm with no yolk sac [YS]; or little or no growth of the gestational sac from the initial scan.

“Sample” as used herein means any biological fluid or tissue that contains the biomarkers. The most suitable samples for use in the methods and with the compositions are blood samples, including serum, plasma, whole blood, and peripheral blood. It is also anticipated that other biological fluids, such as saliva or urine, vaginal or cervical secretions, amniotic fluid, and placental fluid may be used similarly. Such samples may further be diluted with saline, buffer or a physiologically acceptable diluent. Alternatively, such samples are concentrated by conventional means.

By “change in expression” is meant an increased expression level of a selected biomarker, or upregulation of the genes or transcript encoding it in comparison to the reference or control; a decreased expression level of a selected biomarker or a downregulation of the genes or transcript encoding it in comparison to the reference or control; or a combination of certain increased/upregulated and decreased/down regulated biomarkers. The degree of change in target expression can vary with each individual and is subject to variation with each population and days or weeks of the pregnancy. For example, in one embodiment, a large change, e.g., 2-3 fold increase or decrease in a small number of biomarkers, e.g., from 1 to 9 characteristic biomarkers, is statistically significant. In another embodiment, a smaller relative change in about 5, 10, 15, 20, 25, or more biomarkers is statistically significant.

By “target biomarker” or “target biomarker signature” as used herein is meant those proteins/peptides or the genes/transcripts encoding same, the expression of which changes (either in an up-regulated or down-regulated manner) characteristically in the presence of an ectopic pregnancy or non-viable pregnancy from that in an IUP. In one embodiment, at least one target biomarker forms a suitable biomarker signature for use in the methods and compositions. In one embodiment, at least two target biomarkers form a suitable biomarker signature for use in the methods and compositions. Specific biomarker signatures can include any combination of EP biomarkers employing at least one biomarker from (i) to (xi) identified herein and including all 26 biomarkers in Table 2A and all 16 biomarkers in Table 2B, as well as other combinations with the biomarkers. One skilled in the art may readily reproduce the compositions and methods described herein by use of the sequences of the biomarkers, all of which are publicly available from conventional sources, such as GenBank.

The term “microarray” refers to an ordered arrangement of hybridizable array elements, e.g., primers, probes, ligands, on a substrate.

The term “ligand” refers to a molecule that binds to a protein or peptide, and includes antibodies and fragments thereof.

The term “polynucleotide,” when used in singular or plural form, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA: DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

As used herein, “labels” or “reporter molecules” are chemical or biochemical moieties useful for labeling a nucleic acid (including a single nucleotide), polynucleotide, oligonucleotide, or protein ligand, e.g., amino acid, peptide sequence, protein, or antibody. “Labels” and “reporter molecules” include fluorescent agents, chemiluminescent agents, chromogenic agents, quenching agents, radionucleotides, enzymes, substrates, cofactors, inhibitors, radioactive isotopes, magnetic particles, and other moieties known in the art. “Labels” or “reporter molecules” are capable of generating a measurable signal and may be covalently or noncovalently joined to an oligonucleotide or nucleotide (e.g., a non-natural nucleotide) or ligand.

It should be understood that while various embodiments in the specification are presented using “comprising” language, under various circumstances, a related embodiment is also described using “consisting of” or “consisting essentially of” language. It is to be noted that the term “a” or “an”, refers to one or more, for example, “an immunoglobulin molecule,” is understood to represent one or more immunoglobulin molecules. As such, the terms “a” (or “an”), “one or more,” and “at least one” is used interchangeably herein.

Unless defined otherwise in this specification, technical and scientific terms used herein have the same meaning as commonly understood by one of ordinary skill in the art and by reference to published texts, which provide one skilled in the art with a general guide to many of the terms used in the present application.

### Useful Target Biomarkers for Determining Pregnancy Location and Viability

The “targets” of the compositions and methods of these inventions include, in one aspect, the genes, gene fragments, transcripts and the expression products, including the proteins and peptide fragments thereof listed in Tables 2A and 2B. As described in the Examples below, the inventors identified 26 proteins that differed in expression between the conditions of SAB, EP, and IUP. Further analysis resulted in the identification of highly significant protein biomarkers that can reliably distinguish between the conditions of SAB, EP, and IUP. In certain embodiments, compositions and methods for distinguishing ectopic pregnancy from normal intrauterine pregnancy utilize at least one of the biomarkers, or one of the specifically identified isoforms or fragments of known markers. In other embodiments, compositions and methods for distinguishing ectopic pregnancy from normal intrauterine pregnancy utilize at least two or more of the specific target biomarker protein forms identified herein. In still other embodiments, compositions and methods for distinguishing ectopic pregnancy from normal intrauterine pregnancy will utilize at least three or more of the specific target biomarker protein forms identified herein. In still other embodiments, compositions and methods for distinguishing ectopic pregnancy from normal intrauterine pregnancy will utilize at least five or more of the specific target biomarker protein forms identified herein. In still other embodiments, at least 6 or more biomarkers will be employed in the methods and compositions described herein for diagnosis of EP. In still other embodiments, compositions and methods for distinguishing ectopic pregnancy from normal intrauterine pregnancy will utilize at least seven or more of the specific target biomarker protein forms identified herein. In still other embodiments, compositions and methods for distinguishing ectopic pregnancy from normal intrauterine pregnancy will utilize at least eight or more of the specific target biomarker protein forms identified herein. In still other embodiments, compositions and methods for distinguishing ectopic pregnancy from normal intrauterine pregnancy will utilize at least nine or more of the specific target biomarker protein forms identified herein. In still other embodiments, compositions and methods for distinguishing ectopic pregnancy from normal intrauterine pregnancy will utilize at least ten or more of the specific target biomarker protein forms identified herein. In still other embodiments, compositions and methods for distinguishing ectopic pregnancy from normal intrauterine pregnancy will utilize at least eleven or more of the specific target biomarker protein forms identified herein.

In certain embodiments, compositions and methods for distinguishing non-viable pregnancy from viable pregnancy utilize at least one of the novel biomarkers, or one of the specifically identified isoforms or fragments of known markers. In other embodiments, compositions and methods for distinguishing non-viable pregnancy from viable pregnancy utilize at least two or more of the specific target biomarker protein forms identified herein. In still other embodiments, compositions and methods for distinguishing non-viable pregnancy from viable pregnancy will utilize at least three or more of the specific target biomarker protein forms identified herein. In still other embodiments, compositions and methods for distinguishing non-viable pregnancy from viable pregnancy will utilize at least five or more of the specific target biomarker protein forms identified herein. In still other embodiments, at least 6 or more biomarkers will be employed in the methods and compositions described herein for diagnosis of SAB. In still other embodiments, compositions and methods for distinguishing non-viable pregnancy from viable pregnancy will utilize at least 7 or more of the specific target biomarker protein forms identified herein. In still other embodiments, compositions and methods for distinguishing non-viable pregnancy from viable pregnancy will utilize at least 8 or more of the specific target biomarker protein forms identified herein. In still other embodiments, compositions and methods for distinguishing non-viable pregnancy from viable pregnancy will utilize at least 9 or more of the specific target biomarker protein forms identified herein. In still other embodiments, compositions and methods for distinguishing non-viable pregnancy from viable pregnancy will utilize at least 10 or more of the specific target biomarker protein forms identified herein. In still other embodiments, compositions and methods for distinguishing non-viable pregnancy from viable pregnancy will utilize at least 11 or more of the specific target biomarker protein forms identified herein.

In another embodiment a target of the methods and compositions described herein are specific isoforms of a family of related proteins produced by the placenta, called pregnancy specific beta-1 glycoprotein (PSG; also called serum specific protein-1 (SP1)). The inventors determined that certain isoforms not previously associated with EP and SAB can be used as biomarkers/targets in the methods and compositions described herein. Thus, in one embodiment a target for use herein is PSG, isoform 1 (PSG1). The amino acid sequence and nucleic acid sequence for PSG1 are publicly available, see, e.g., GENBANK Accession No. NM 006905.3 and XP_005259122.1.

In another embodiment, a target for use herein is PSG, isoform 3 (PSG3). The amino acid sequence and nucleotide sequence for PSG3 are publicly available, see, e.g., GENBANK Accession No. NM_021016.4 and NP_066296.2.

In another embodiment a target for use herein is PSG, isoform 9 (PSG9). The amino acid sequence and nucleotide sequence for PSG9 are publicly available, see, e.g., GENBANK Accession No. NP_002775.3 and NM 002784.5.

In another embodiment a target of the methods and compositions described herein is CGA, known as Glycoprotein hormones alpha chain. The amino acid sequence and nucleic acid sequence for CGA are publicly available, see, e.g., GENBANK Accession No. NP_001239312.1 and NM_001252383.2.

In one embodiment a target of the methods and compositions described herein is hCG, known as beta human chorionic gonadotropin. The amino acid sequence and nucleic acid sequence for hCG are publicly available, see, e.g., GENBANK Accession No. V00518.1 and CAA23777.1.

In another embodiment a target of the methods and compositions described herein is PAPPA, known as Pappalysin-1 (also known as pregnancy-associated plasma protein A). The amino acid sequence and nucleic acid sequence for PAPPA are publicly available, see, e.g., GENBANK Accession No. NP_002572.2 and NM_002581.5.

In one embodiment a target of the methods and compositions described herein is sFLT, known as soluble fms-like tyrosine kinase-1. The amino acid sequence and nucleic acid sequence for sFLT are publicly available, see, e.g., GENBANK Accession No. NM 002019.4 and NP_002010.2.

In one embodiment a target of the methods and compositions described herein is TFPI2, known as tissue factor pathway inhibitor 2. The amino acid sequence and nucleic acid sequence for TFPI2 are publicly available, see, e.g., GENBANK Accession No. AF217542.1 and AAK13254.1.

In one embodiment a target of the methods and compositions described herein is ADAM12, known as disintegrin and metalloproteinase domain-containing protein 12. The amino acid sequence and nucleic acid sequence for ADAM12 are publicly available, see, e.g., GENBANK Accession No. NP_001275902.1 and NM_001288973.2.

In one embodiment a target of the methods and compositions described herein is GDF15, known as growth-differentiation faction-15. The amino acid sequence and nucleic acid sequence for GDF15 are publicly available, see, e.g., GENBANK Accession No. NM 004864.4 and NP_004855.2.

In one embodiment a target of the methods and compositions described herein is PRG, known as progesterone. The amino acid sequence and nucleic acid sequence for PRG are publicly available, see, e.g., GENBANK Accession NM_001202474.3 and NP_001189403.1.

In one embodiment a target of the methods and compositions described herein is ANGPT2, known as angiopoietin 2. The amino acid sequence and nucleic acid sequence for ANGPT2 are publicly available, see, e.g., GENBANK Accession NP_001138.1 and NM 001147.3.

In one embodiment a target of the methods and compositions described herein is GD, or PAEP, also known as glycodelin or progestogen-associated endometrial protein. The amino acid sequence and nucleic acid sequence for glycodelin are publicly available, see, e.g., GENBANK Accession NP_001018059.1 and NM_001018049.3.

In one embodiment a target of the methods and compositions described herein is activin A. The amino acid sequence and nucleic acid sequence for activin A are publicly available, see, e.g., GENBANK Accession KAI4036582.1 and NM 001105.5.

In one embodiment a target of the methods and compositions described herein is IGFBP1, known as insulin-like growth factor binding protein 1. The amino acid sequence and nucleic acid sequence for IGFBP1 are publicly available, see, e.g., GENBANK Accession No. NP_000587.1 and NM_000596.4.

In another embodiment a target of the methods and compositions described herein is KISS1, known as kissspeptin. The amino acid sequence and nucleic acid sequence for KISS1 are publicly available, see, e.g., GENBANK Accession No. NP_002247.3 and NM 002256.4.

In another embodiment a target of the methods and compositions described herein is PARVB, known as beta parvin. The amino acid sequence and nucleic acid sequence for PARVB are publicly available, see, e.g., GENBANK Accession No. NM_001003828.3 and XP_024308003.1.

In another embodiment a target of the methods and compositions described herein is EHD3, known as EH domain-containing protein 3. The amino acid sequence and nucleic acid sequence for EHD3 are publicly available, see, e.g., GENBANK Accession No. NP_055415.1 and NM 014600.3.

In another embodiment a target of the methods and compositions described herein is HE4, known as WAP four-disulfide core domain 2. The amino acid sequence and nucleic acid sequence for HE4 are publicly available, see, e.g., GENBANK Accession No. AAH46106.1 and BC046106.1.

In another embodiment a target of the methods and compositions described herein is QSOX2, known as quiescin sulfhydryl oxidase 2. The amino acid sequence and nucleic acid sequence for QSOX2 are publicly available, see, e.g., GENBANK Accession No. NP_859052.3 and NM_181701.4.

In still other embodiments, the target for use in the methods and compositions described herein can include various combinations of these target biomarkers and/or fragments thereof.

In another embodiment a target combination, protein biomarker signature for use herein includes other known EP biomarkers in combination with the markers above (e.g., choriogonadotropin subunit beta precursor (CGB), chorionic somatomammotropin hormone precursor (CSH1).

Among desirable biomarker signatures for the discrimination of pregnancy viability are signatures including at least one biomarker, at least two biomarkers, or all three biomarkers selected from PSG3, PAPPA, and CGA. In one embodiment, all three biomarkers are included.

Among desirable biomarkers for the discrimination of pregnancy location are signatures including at least one biomarker, at least two biomarkers, at least three biomarkers, or all four biomarkers selected from PSG3, PSG1, sFLT, and TFPI2.

Still other desirable biomarker signatures for the discrimination of pregnancy viability are signatures including at least one biomarker, at least two biomarkers, at least 3, 4, 5, 6, 7, 8, 9, 10, or all 11 biomarkers selected from PSG1, sFLT, GDF15 PSG9, hCG, CG-Alpha, Adam 12, ANGPT2, TFPI2, PSG3, and PRG.

Further desirable biomarker signatures for the discrimination of pregnancy location are signatures including at least one biomarker, at least two biomarkers, at least three biomarkers, at least five biomarkers or all six biomarkers selected from pregnancy-specific beta-1-glycoprotein 9 (PSG9), pregnancy-specific beta-1-glycoprotein 1 (PSG1), insulin-like growth factor binding protein 1 (IGFBP1), kisspeptin (KISS1), pregnancy-specific beta-1-1glycoprotein 3 (PSG3), and beta parvin (PARVB). Other suitable biomarker signatures include combinations of at least one of the above noted 6 biomarkers with at least one of the following biomarkers: EH domain-containing protein 3 (EHD3), WAP four-disulfide core domain protein 2 (HE4), and quiescin sulfhydryl oxidase 2 (QSOX2). Still another embodiment of a biomarker signature contains all of the above-recited biomarkers. In another embodiment, a biomarker signature contains at least one, at least 5, at least 10, at least 15, at least 25, at least 30, at least 35, at least 40, or all 42, additional biomarkers from those identified in Table 2A and 2B.

Alternatively, desirable biomarker signatures for the discrimination of pregnancy location and/or viability are signatures including at least one biomarker, at least two biomarkers, at least three biomarkers, at least five biomarkers, at least ten or all eleven biomarkers selected from disintegrin and metalloproteinase domain-containing protein 12 (ADAM12), Glycoprotein hormones alpha chain (CGA), beta human chorionic gonadotropin (hCG), Pappalysin-1 (PAPPA), pregnancy-specific beta-1-glycoprotein 1 (PSG1), pregnancy-specific beta-1-1glycoprotein 3 (PSG3), pregnancy-specific beta-1-glycoprotein 9 (PSG9), soluble fms-like tyrosine kinase-1 (sFLT), growth-differentiation factor-15 (GDF15), progesterone (PRG), and tissue factor pathway inhibitor 2 (TFPI2). In another embodiment, a biomarker signature contains at least one, at least 5, at least 10, at least 15, at least 25, at least 30, at least 35, at least 40, or all 42, additional biomarker from those identified in Table 2A and 2B.

Among desirable biomarker signatures for the discrimination of pregnancy viability are signatures including at least one biomarker, at least two biomarkers, at least three biomarkers, at least five biomarkers or all seven biomarkers selected from pregnancy-specific beta-1-glycoprotein 9 (PSG9), pregnancy-specific beta-1-glycoprotein 1 (PSG1), EH domain-containing protein 3 (EHD3), WAP four-disulfide core domain protein 2 (HE4), kisspeptin (KISS1), pregnancy-specific beta-1-1glycoprotein 3 (PSG3), and quiescin sulfhydryl oxidase 2 (QSOX2). Other suitable biomarker signatures include combinations of at least one of the above noted biomarkers with at least one of the following biomarkers: beta parvin (PARVB) and insulin-like growth factor binding protein 1 (IGFBP1). Still another embodiment of a biomarker signature contains all of the above-recited biomarkers. In another embodiment, a biomarker signature contains at least one, at least 5, at least 10, at least 15, at least 25, at least 30, at least 35, at least 40, or all 42, additional biomarker from those identified in Tables 2A and 2B.

### Diagnostic Reagents and Kits

### Labeled or Immobilized Biomarkers or Peptides

In one embodiment, diagnostic reagents for use in the methods of determining pregnancy location includes one target biomarker identified herein, associated with a detectable label or portion of a detectable label system. In another embodiment, a diagnostic reagent includes one target biomarker herein, immobilized on a substrate. In still another embodiment, combinations of such labeled or immobilized biomarkers are suitable reagents and components of a diagnostic kit. Among such immobilized or labeled biomarkers are those selected from the biomarkers:
i. pregnancy specific beta-1-glycoprotein 9 (PSG9),
ii. pregnancy specific beta-1 glycoprotein 1 (PSG1),
iii. insulin-like growth factor binding protein 1 (IGFBP1),
iv. kisspeptin (KISS1),
v. pregnancy specific beta-1 glycoprotein 3 (PSG3), and
vi. beta-parvin (PARVB).

In another aspect, suitable embodiments of such labeled or immobilized reagents include at least one, 2, 3, 4, 5, or all 6 of biomarkers (i) to (vi) or their unique peptide fragments therein. In another aspect, the immobilized or labeled biomarkers further comprises:
vii. EH domain-containing protein 3 (EHD3);
viii. WAP four-disulfide core domain protein 2 (HE4); and
ix. quiescin sulfhydryl oxidase 2 (QSOX2).

In another aspect, other suitable embodiments of such labeled or immobilized reagents include an additional at least one, 2, or 3 of biomarkers (vii) through (ix) or their unique peptide fragments therein.

In another embodiment, diagnostic reagents for use in the methods of determining pregnancy viability includes one target biomarker identified herein, associated with a detectable label or portion of a detectable label system. In another embodiment, a diagnostic reagent includes one target biomarker herein, immobilized on a substrate. In still another embodiment, combinations of such labeled or immobilized biomarkers are suitable reagents and components of a diagnostic kit. Among such immobilized or labeled biomarkers are those selected from the biomarkers:
i. pregnancy specific beta-1-glycoprotein 9 (PSG9),
ii. pregnancy specific beta-1 glycoprotein 3 (PSG3),
iii. EH domain-containing protein 3 (EHD3),
iv. kisspeptin (KISS1),
v. WAP four-disulfide core domain protein 2 (HE4),
vi. quiescin sulfhydryl oxidase 2 (QSOX2); and
vii. pregnancy specific beta-1 glycoprotein 1 (PSG1).

In another aspect, suitable embodiments of such labeled or immobilized reagents include at least one, 2, 3, 4, 5, 6, or all 7 of biomarkers (i) to (vii) or their unique peptide fragments therein. In another aspect, the immobilized or labeled biomarkers further comprises:
viii. insulin-like growth factor binding protein 1 (IGFBP1); and
ix. beta-parvin (PARVB).

In another aspect, other suitable embodiments of such labeled or immobilized reagents include an additional at least one of biomarkers (viii) through (ix) or their unique peptide fragments therein.

In another embodiment, diagnostic reagents for use in the methods of determining pregnancy location and/or viability includes one target biomarker identified herein, associated with a detectable label or portion of a detectable label system. In another embodiment, a diagnostic reagent includes one target biomarker herein, immobilized on a substrate. In still another embodiment, combinations of such labeled or immobilized biomarkers are suitable reagents and components of a diagnostic kit. Among such immobilized or labeled biomarkers are those selected from the biomarkers:
i. disintegrin and metalloproteinase domain-containing protein 12 (ADAM12),
ii. Glycoprotein hormones alpha chain (CGA),
iii. beta human chorionic gonadotropin (hCG),
iv. Pappalysin-1 (PAPPA),
v. pregnancy-specific beta-1-glycoprotein 1 (PSG1),
vi. pregnancy-specific beta-1-1glycoprotein 3 (PSG3),
vii. pregnancy-specific beta-1-glycoprotein 9 (PSG9),
viii. soluble fms-like tyrosine kinase-1 (sFLT),
ix. growth-differentiation faction-15 (GDF15), and
x. progesterone (PRG), and
xi. tissue factor pathway inhibitor 2 (TFPI2).

In another aspect, suitable embodiments of such labeled or immobilized reagents include at least one, 2, 3, 4, 5, 6, 7, 8, 9, 10, or all 11 of biomarkers (i) to (xi) or their unique peptide fragments therein.

In certain embodiments, the diagnostic reagents detect pregnancy viability and the peptides or proteins are (vi) PSG3, (vii) PSG9, (ii) CGA, (iii) hCG, (ix) GDF15 and (x) PRG. In certain embodiments, the diagnostic reagents detect pregnancy location and the peptides or proteins are (v) PSG1, (viii) sFLT, (ix) GDF15, (vii) PSG9, (iii) hCG, and (ii) CGA. In certain embodiments, the diagnostic reagents detect pregnancy viability and the peptides or proteins are (vi) PSG3, and (ii) CGA. In certain embodiments, the diagnostic reagents detect pregnancy viability or pregnancy location and the peptides or proteins are PSG1, sFLT, GDF15 PSG9, hCG, CG-Alpha, Adam 12, ANGPT2, TFPI2, PSG3, and PRG.

In certain embodiments, the diagnostic reagents detect pregnancy location and the peptide or protein is (viii) sFLT. In certain embodiments, the diagnostic reagents detect pregnancy location and the peptide or protein is (v) PSG1. In certain embodiments, the diagnostic reagents detect pregnancy location and the peptides or proteins are (vi) PSG3, and (xi) TFPI2. In certain embodiments, the diagnostic reagents detect pregnancy location and the peptides or proteins are (viii) sFLT, (vi) PSG3, and (xi) TFPI2. In certain embodiments, the diagnostic reagents detect pregnancy location and the peptides or proteins are (i) ADAM12, (ii) CGA, (iii) hCG, (iv) PAPPA, (v) PSG1, (vi) PSG3, (vii) PSG9, (viii) sFLT, (ix) GDF15, (x) PRG, and (xi) TFPI2. In certain embodiments, the diagnostic reagents detect pregnancy location and the peptides or proteins are (i) ADAM12, (ii) CGA, (iii) hCG, (iv) PAPPA, (v) PSG1, (vi) PSG3, (vii) PSG9, (viii) sFLT, (ix) GDF15, (x) PRG, and (xi) TFPI2.

In certain embodiments, the diagnostic reagents detect pregnancy viability and the peptide or protein is (vi) PSG3. In certain embodiments, the diagnostic reagents detect pregnancy viability and the peptides or proteins are (iv) PAPPA and (vi) PSG3. In certain embodiments, the diagnostic reagents detect pregnancy viability and the peptides or proteins are (ii) CGA and (iv) PAPPA. In certain embodiments, the diagnostic reagents detect pregnancy viability and the peptides or proteins are (ii) CGA, (iv) PAPPA, and (vi) PSG3. In certain embodiments, the diagnostic reagents detect pregnancy viability and the peptides or proteins are (vii) PSG9 and (vi) PSG3. In certain embodiments, the diagnostic reagents detect pregnancy viability and the peptides or proteins are (ii) CGA, (iii) hCG, (vi) PSG3, (vii) PSG9, (ix) GDF15, and (x) PRG. In certain embodiments, the diagnostic reagents detect pregnancy viability and the peptides or proteins are (i) ADAM12, (ii) CGA, (iii) hCG, (iv) PAPPA, (v) PSG1, (vi) PSG3, (vii) PSG9, (viii) sFLT, (ix) GDF15, and (x) PRG.

Any combination of labeled or immobilized biomarkers can be assembled in a diagnostic kit for the purposes of diagnosing EP or SAB. For example, one embodiment of a diagnostic kit includes labeled or immobilized reagents (i) through (v). Still other components of the biomarker signatures, associated with detectable labels or immobilized on substrates provide additional diagnostic kits. Still other components of the biomarker signatures are 5 labeled or immobilized biomarkers or fragments thereof as listed in Tables 2A and 2B.

TABLE 2A
Biomarker Molecules

| Biomarker | | |
|---|---|---|
| (Gene Name) | Protein Description | Biologic Plausibility |
| PSG9 | Pregnancy-specific | Trophoblast function, cell adhesion, |
| | beta-1-glycoprotein 9 | hemostasis |
| PSG1 | Isoform 2 of | Trophoblast function, cell adhesion, |
| | Pregnancy-specific beta-1- | hemostasis |
| | glycoprotein 1 | |
| IGFBP1 | Insulin-like growth factor | Endometrial function, implantation, |
| | binding protein 1 | cell migration |
| KISS1 | Metastasis- suppressor of KISS- | Trophoblast invasion and migration, |
| | 1 (Kisspeptin) | cell adhesion, |
| PSG3 | Pregnancy-specific beta-1- | Trophoblast function, cell adhesion, |
| | glycoprotein 3 | hemostasis |
| PARVB | Parvin, beta isoform | Cell adhesion, differentiation, actin |
| | a, beta parvin | binding |
| MMP9 | Matrix Metallopeptidase 9 | Hematopoietic endopeptidase, |
| | | hemostasis |
| HE4 | WAP four-disulfide core domain | Endopeptidase inhibitor, |
| | protein 2 (WFDC2) | endometrial and fallopian tube |
| | | glandular cell function |
| ISM2 | Isthmin 2 (Thrombospondin, | Trophoblast function, endothelial |
| | type I domain containing 3 | protein |
| | isoform 1) | |
| PAEP | Progestogen associated | Endometrial and fallopian tube |
| | endometrial protein, Placental | function, cell processes, small |
| | protein 14, Glycodelin | molecular binding |
| CLIC1 | Chloride Intracellular | Chloride channel ion transport, |
| | Channel 1 | trophoblast function |
| QSOX2 | Quiescin Sulfhydryl Oxidase 2 | Oxidoreductase, trophoblast |
| | | function |
| MYLK | Myosin Light Chain Kinase | Smooth muscle contraction |
| NOTUM | Palmitoleoyl-protein | Trophoblast function, cell signaling |
| | carboxylesterase NOTUM | (Wnt pathway) |
| WDR1 | WD repeat-domain | Trophoblast, fallopian tube, and |
| | containing protein 1 | endometrial gland actin binding |
| EHD3 | EH domain- | Trophoblast function, cell structure |
| | containing protein 3 | regulation, protein transport |
| PLEK | Pleckstrin | Cytoskeleton & endometrial cell |
| | | signaling, hemostasis |
| INHBB | Activin B | Cell processes and ligand binding, |
| | | growth factor, apoptosis |

TABLE 2B
Additional Biomarkers

sFLT
GDF15
hCG
CG-ALPHA
Adam12
SIGLEC-6
ANGPT2
TFPI2
Activin A
PLGF
PAPPA
PRG
HAGH
ELAFIN
Fibronectin
OPN

For these reagents, the labels may be selected from among many known diagnostic labels, including those described above. Similarly, the substrates for immobilization may be any of the common substrates, glass, plastic, a microarray, a microfluidics card, a chip or a chamber.

Labeled or Immobilized Ligands that Bind the Biomarkers or Peptides

In another embodiment, the diagnostic reagent is a ligand that binds to a biomarker recited above or a unique peptide thereof. Such a ligand desirably binds to a protein biomarker or a unique peptide contained therein, and can be an antibody which specifically binds a single biomarker described above, or a unique peptide in that single biomarker. Various forms of antibody, e.g., polyclonal, monoclonal, recombinant, chimeric, as well as fragments and components (e.g., CDRs, single chain variable regions, etc.) may be used in place of antibodies. The ligand itself may be labeled or immobilized.

In another aspect, suitable embodiments of such labeled or immobilized reagents include at least one, 2, 3, 4, 5, 6, 7, 8, 9, 10, or 11 ligands. Each ligand binds to a single biomarker or their unique peptide fragments therein. In another aspect, other suitable embodiments of such labeled or immobilized reagents include an additional at least one, 2, or 3 ligands, wherein each ligand binds to a single biomarker or their unique peptide fragments therein.

Any combination of labeled or immobilized biomarker-binding ligands can be assembled in a diagnostic kit for the purposes of diagnosing EP or SAB. For example, one embodiment of a diagnostic kit includes labeled or immobilized reagents that bind to biomarkers (i) through (v).

Labeled or Immobilized Polynucleotide/Oligonucleotides that Hybridize to Genes, Gene Fragments, Gene Transcripts of Other Sequences Encoding the Biomarkers or Peptides

In another embodiment, the diagnostic reagent is a polynucleotide or oligonucleotide sequence that hybridizes to gene, gene fragment, gene transcript or nucleotide sequence encoding a biomarker of any one or more of the biomarkers described above or encoding a unique peptide thereof. Such a polynucleotide/oligonucleotide can be a probe or primer, and may itself be labeled or immobilized. In another aspect, suitable embodiments of such labeled or immobilized reagents include at least one, 2, 3, 4, 5, 6, 7, 8, 9, 10, or 11 polynucleotide/oligonucleotide. Each polynucleotide/oligonucleotide hybridizes to a gene, gene fragment, gene transcript or expression product encoding a single biomarker or their unique peptide fragments therein. In another aspect, other suitable embodiments of such labeled or immobilized reagents include an additional at least one, 2, 3, 4 or 5 polynucleotide/oligonucleotides, wherein each sequence hybridizes to a gene, gene fragment, gene transcript of expression product encoding a single biomarker or their unique peptide fragments therein. In certain embodiments, the diagnostic reagent hybridizes to mRNA.

Any combination of labeled or immobilized biomarker-hybridizable sequences can be assembled in a diagnostic kit for the purposes of diagnosing EP or SAB. For example, one embodiment of a diagnostic kit includes labeled or immobilized reagents that hybridize to at least one of the biomarkers described above. Another embodiment of a diagnostic kit includes labeled or immobilized reagents that hybridize all of the biomarkers described above. Still other components of the many biomarker signatures that may be formed by various combinations of polynucleotide/oligonucleotide sequences that hybridize to the biomarkers described above, or their unique fragments associated with detectable labels or immobilized on substrates provide additional diagnostic kits. In one embodiment, these polynucleotide or oligonucleotide reagent(s) are part of a primer-probe set, and the kit comprises both primer and probe. Each said primer-probe set amplifies a different gene, gene fragment or gene expression product that encodes a different biomarker of any combination of the markers described above, optionally including one or more additional biomarkers. In still another embodiment, additional polynucleotide or oligonucleotide sequences in the diagnostic reagent or kit, hybridize to a gene, gene fragment, gene transcript or expression product identified in Tables 2A and 2B.

For use in the compositions the PCR primers and probes are preferably designed based upon intron sequences present in the biomarker gene(s) to be amplified selected from the gene expression profile. The design of the primer and probe sequences is within the skill of the art once the particular gene target is selected. The particular methods selected for the primer and probe design and the particular primer and probe sequences are not limiting features of these compositions. A ready explanation of primer and probe design techniques available to those of skill in the art is summarized in U.S. Pat. No. 7,081,340, with reference to publicly available tools such as DNA BLAST software, the Repeat Masker program (Baylor College of Medicine), Primer Express (Applied Biosystems); MGB assay-by-design (Applied Biosystems); Primer3 (Steve Rozen and Helen J. Skaletsky (2000) Primer3 on the WWW for general users and for biologist programmers and other publications.

In general, optimal PCR primers and probes used in the compositions described herein are generally 17-30 bases in length, and contain about 20-80%, such as, for example, about 50-60% G+C bases. Melting temperatures of between 5° and 80° C., e.g., about 50 to 70° C. are typically preferred.

Thus, a composition for diagnosing ectopic pregnancy or non-viable pregnancy in a mammalian subject as described herein can be a kit containing multiple reagents or one or more individual reagents. For example, one embodiment of a composition includes a substrate upon which the biomarkers, polynucleotides or oligonucleotides, or ligands are immobilized. In another embodiment, the composition is a kit also contains optional detectable labels, immobilization substrates, optional substrates for enzymatic labels, as well as other laboratory items.

The compositions based on the biomarkers selected from Tables 2A or 2B or described herein, optionally associated with detectable labels, can be presented in the format of a microfluidics card, a chip or chamber, or a kit adapted for use with the assays described in the Examples, ELISAs or PCR, RT-PCR or Q PCR techniques described herein.

The selection of the ligands, poly/oligonucleotide sequences, their length, suitable labels and substrates used in the composition are routine determinations made by one of skill in the art in view of the teachings of which biomarkers form signature suitable for the diagnosis of ectopic pregnancy or non-viable pregnancy.

### Methods of Determining Pregnancy Location and Viability

### Protein Assays

In one embodiment, a method for diagnosing an ectopic pregnancy or a non-viable pregnancy in a female mammalian subject includes measuring in a biological fluid sample of the subject the expression level of a protein or peptide fragment thereof selected from at least one biomarker described above. Alternatively, the method includes measuring a combination of two or more biomarkers described above. The method further involves comparing the subject's expression level of the selected biomarker or biomarker fragment with the level of the same protein or peptide in the biological fluid of a reference or control female mammalian subject having a normal intrauterine pregnancy (IUP). Changes in expression of the subject's selected biomarker protein or peptide fragment from those of the reference or control correlates with a diagnosis of ectopic pregnancy.

In another embodiment, the above method further includes measuring in the biological fluid sample of the subject the expression level of an additional biomarker protein or peptide fragment. In another embodiment, the above method further includes measuring in the biological fluid sample of the subject the expression level of two or more additional biomarker protein or peptide fragments.

In another embodiment, the above method further includes measuring in the biological fluid sample of the subject the expression level of an additional biomarker protein or peptide fragment of a biomarker identified in Tables 2A and 2B.

In certain embodiments, the method of determining pregnancy location and/or viability includes measuring in the biological fluid sample of the subject the expression level of a biomarker protein or peptide fragment of a biomarker selected from:
i. disintegrin and metalloproteinase domain-containing protein 12 (ADAM12),
ii. Glycoprotein hormones alpha chain (CGA),
iii. beta human chorionic gonadotropin (hCG),
iv. Pappalysin-1 (PAPPA),
v. pregnancy-specific beta-1-glycoprotein 1 (PSG1),
vi. pregnancy-specific beta-1-1glycoprotein 3 (PSG3),
vii. pregnancy-specific beta-1-glycoprotein 9 (PSG9),
viii. soluble fms-like tyrosine kinase-1 (sFLT),
ix. growth-differentiation faction-15 (GDF15), and
x. progesterone (PRG), and
xi. tissue factor pathway inhibitor 2 (TFPI2).

In certain embodiments, the method includes measuring the expression level of at least one, 2, 3, 4, 5, 6, 7, 8, 9, 10, or all 11 of biomarkers (i) to (xi) or their unique peptide fragments therein.

In certain embodiments, the method detects pregnancy viability and the biomarkers are (vi) PSG3, (vii) PSG9, (ii) CGA, (iii) hCG, (ix) GDF15 and (x) PRG. In certain embodiments, the method detects pregnancy location and the biomarkers are (v) PSG1, (viii) sFLT, (ii), (ix) GDF15, (vii) PSG9, (iii) hCG, and (ii) CGA. In certain embodiments, the method detects pregnancy viability and the biomarkers are (vi) PSG3, and (ii) CGA.

In certain embodiments, the method detects pregnancy location and the biomarker is (viii) sFLT. In certain embodiments, the method detects pregnancy location and the biomarker is (v) PSG1. In certain embodiments, the method detects pregnancy location and the biomarkers are (vi) PSG3, and (xi) TFPI2. In certain embodiments, the method detects pregnancy location and the biomarkers are (viii) sFLT, (vi) PSG3, and (xi) TFPI2. In certain embodiments, the method detects pregnancy location and the biomarkers are (i) ADAM12, (ii) CGA, (iii) hCG, (iv) PAPPA, (v) PSG1, (vi) PSG3, (vii) PSG9, (viii) sFLT, (ix) GDF15, (x) PRG, and (xi) TFPI2. In certain embodiments, the method detects pregnancy location and the biomarkers are (i) ADAM12, (ii) CGA, (iii) hCG, (iv) PAPPA, (v) PSG1, (vi) PSG3, (vii) PSG9, (viii) sFLT, (ix) GDF15, (x) PRG, and (xi) TFPI2.

In certain embodiments, the method detects pregnancy viability and the biomarker is (vi) PSG3. In certain embodiments, the method detects pregnancy viability and the biomarkers are (iv) PAPPA and (vi) PSG3. In certain embodiments, the method detects pregnancy viability and the biomarkers are (ii) CGA and (iv) PAPPA. In certain embodiments, the method detects pregnancy viability and the biomarkers are (ii) CGA, (iv) PAPPA, and (vi) PSG3. In certain embodiments, the method detects pregnancy viability and the biomarkers are (vii) PSG9 and (vi) PSG3. In certain embodiments, the method detects pregnancy viability and the biomarkers are (ii) CGA, (iii) hCG, (vi) PSG3, (vii) PSG9, (ix) GDF15, and (x) PRG. In certain embodiments, the method detects pregnancy viability and the biomarkers are (i) ADAM12, (ii) CGA, (iii) hCG, (iv) PAPPA, (v) PSG1, (vi) PSG3, (vii) PSG9, (viii) sFLT, (ix) GDF15, and (x) PRG.

In one embodiment, a method of diagnosing pregnancy location in a pregnant subject, the method comprising obtaining a measurement of Activin A in a sample from the subject, wherein, when the sample contains less than or equal to 275.1 pg/mL Activin A, diagnosing the pregnancy is an ectopic pregnancy, and when the sample contains greater than 275 pg/mL Activin A, further obtaining a measurement of glycodelin in a sample from the subject, wherein, when the sample contains less than or equal to 0.17 ng/ml or greater than 1.39 ng/ml glycodelin, the pregnancy is an intrauterine pregnancy, and when the sample contains from 0.17 ng/mL to 1.39 ng/mL glycodelin, the pregnancy is an ectopic pregnancy.

In one embodiment, a method of diagnosing pregnancy location in a pregnant subject, the method comprising obtaining a measurement of Activin A in a sample from the subject, wherein, when the sample contains greater than 179.45 pg/mL Activin A, diagnosing the pregnancy is intrauterine, and when the sample contains less than or equal to 179.45 pg/mL Activin A, further obtaining a measurement of ADAM 12 in a sample from the subject, wherein, when the sample contains less than or equal to 0.42 ng/ml or greater than 0.89 ng/ml ADAM 12, the pregnancy is an ectopic pregnancy, and when the sample contains from 0.42 ng/ml to 0.89 ng/mL ADAM12, the pregnancy is intrauterine.

In another embodiment, a method of diagnosing pregnancy location in a pregnant subject, the method comprises obtaining a measurement of sFLT in a sample from the subject, wherein, when the sample contains less than 598.0 pg/mL sFLT, diagnosing the patient with an ectopic pregnancy.

In another embodiment, a method of diagnosing pregnancy location in a pregnant subject, the method comprises obtaining a measurement of PSG1 in a sample from the subject, wherein, when the sample contains less than 22.5 ng/mL PSG1 in a sample, diagnosing the patient with an ectopic pregnancy.

In another embodiment, a method of diagnosing pregnancy location in a pregnant subject, the method comprises obtaining a measurement of PSG3 and TFPI2 in a sample from the subject, wherein, when the sample contains less than 34.16 ng/mL PSG3 and less than 282.5 pg/mL TFPI2 in a sample, diagnosing the patient with an ectopic pregnancy.

In another embodiment, a method of diagnosing pregnancy location in a pregnant subject, the method comprises obtaining a measurement of sFLT, PSG3, and TFPI2 in a sample from the subject, wherein, when the sample contains less than 598.0 pg/mL sFLT and less than 34.16 ng/mL PSG3 and less than 282.5 pg/mL TFPI2 in a sample, diagnosing the patient with an ectopic pregnancy.

In one embodiment, a method of diagnosing pregnancy viability in a pregnant subject, the method comprising obtaining a measurement of progesterone in a sample from the subject, wherein, when the sample contains less than 7.99 ng/mL progesterone, diagnosing the pregnancy is intrauterine, and when the sample contains greater than or equal to 7.99 ng/ml progesterone, diagnosing the pregnancy as nonviable.

In one embodiment, a method of diagnosing pregnancy viability in a pregnant subject, the method comprising obtaining a measurement of progesterone in a sample from the subject, wherein, when the sample contains less than or equal to 17.28 ng/mL progesterone, diagnosing the pregnancy is nonviable, and when the sample contains greater than 17.28 ng/ml progesterone, further obtaining a measurement of PAPPA in a sample from the subject, wherein, when the sample contains greater than 0.22 ng/mL PAPPA, the pregnancy is an intrauterine, and when the sample contains less than or equal to 0.22 ng/mL PAPPA, further obtaining a measurement of progesterone, wherein, when the sample contains less than or equal to 17.84 ng/ml progesterone, the pregnancy is intrauterine, and when the sample contains greater than 17.84 ng/ml progesterone, the pregnancy is nonviable.

In another embodiment, a method of diagnosing pregnancy viability in a pregnant subject, the method comprises obtaining a measurement of PSG3 in a sample from the subject, wherein, when the sample contains less than 16.7 ng/mL PSG3, diagnosing the patient with a nonviable pregnancy.

In another embodiment, a method of diagnosing pregnancy viability in a pregnant subject, the method comprises obtaining a measurement of PAPPA and PSG3 in a sample from the subject, wherein, when the sample contains less than 25.57 ng/ml PSG3 or greater than 29.57 ng/ml PSG3 and greater than 0.52 ng/ml PAPPA in a sample, diagnosing the patient with a nonviable pregnancy.

In another embodiment, a method of diagnosing pregnancy viability in a pregnant subject, the method comprises obtaining a measurement of CGA and PAPPA in a sample from the subject, wherein, when the sample contains less than 10960 mIU/mL CGA or greater than 10960 mIU/mL CGA in a sample and greater than 0.585 ng/ml PAPPA, diagnosing the patient with a nonviable pregnancy.

In another embodiment, a method of diagnosing pregnancy viability in a pregnant subject, the method comprises obtaining a measurement of PSG3, PAPPA, and CGA in a sample from the subject, wherein, when the sample contains less than 16.7 pg/mL PSG3 and less than 10960 mIU/ml CGA or greater than 10960 mIU/mL CGA and greater than 0.585 ng/ml PAPA CGA in a sample, diagnosing the patient with a nonviable pregnancy.

In this diagnostic method, a change in expression level of one or more of the selected biomarker proteins or peptide fragment in comparison to the IUP control reference may be an increase or decrease in the expression levels of the individual biomarkers. This method may employ any of the suitable diagnostic reagents or kits or compositions described above.

The measurement of the EP and/or SAB biomarkers in the biological sample may employ any suitable ligand, e.g., antibody (or antibody to any second biomarker) to detect the EP and/or SAB biomarker protein. Such antibodies may be presently extant in the art or presently used commercially, such as those available as part of commercial antibody ELISA assay kits or that may be developed by techniques now common in the field of immunology. As used herein, the term “antibody” refers to an intact immunoglobulin having two light and two heavy chains or any fragments thereof. Thus, a single isolated antibody or fragment may be a polyclonal antibody, a high affinity polyclonal antibody, a monoclonal antibody, a synthetic antibody, a recombinant antibody, a chimeric antibody, a humanized antibody, or a human antibody. The term “antibody fragment” refers to less than an intact antibody structure, including, without limitation, an isolated single antibody chain, a single chain Fv construct, a Fab construct, a light chain variable or complementarity determining region (CDR) sequence, etc. A recombinant molecule bearing the binding portion of an EP biomarker antibody, e.g., carrying one or more variable chain CDR sequences that bind e.g., ISM2, may also be used in a diagnostic assay. As used herein, the term “antibody” may also refer, where appropriate, to a mixture of different antibodies or antibody fragments that bind to the selected biomarker. Such different antibodies may bind to different biomarkers or different portions of the same EP or SAB biomarker protein than the other antibodies in the mixture. Such differences in antibodies used in the assay may be reflected in the CDR sequences of the variable regions of the antibodies. Such differences may also be generated by the antibody backbone, for example, if the antibody itself is a non-human antibody containing a human CDR sequence, or a chimeric antibody or some other recombinant antibody fragment containing sequences from a non-human source. Antibodies or fragments useful in the method of this invention may be generated synthetically or recombinantly, using conventional techniques or may be isolated and purified from plasma or further manipulated to increase the binding affinity thereof. It should be understood that any antibody, antibody fragment, or mixture thereof that binds one of the biomarkers or a particular sequence of the selected EP or SAB biomarkers may be employed in the methods of the present invention, regardless of how the antibody or mixture of antibodies was generated.

Similarly, the antibodies may be tagged or labeled with reagents capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. Most desirably, the label is detectable visually, e.g., colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to provide a visual signal indicative of the presence of the resulting selected biomarker-antibody complex in applicable assays. Still other labels include fluorescent compounds, radioactive compounds or elements. Preferably, an anti-biomarker antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

Detectable labels for attachment to antibodies useful in diagnostic assays of this invention may be easily selected from among numerous compositions known and readily available to one skilled in the art of diagnostic assays. The EP or SAB biomarker-antibodies or fragments useful in this invention are not limited by the particular detectable label or label system employed. Thus, selection and/or generation of suitable EP or SAB biomarker antibodies with optional labels for use in this invention is within the skill of the art, provided with this specification, the documents incorporated herein, and the conventional teachings of immunology.

Similarly the particular assay format used to measure the selected EP or SAB biomarker in a biological sample may be selected from among a wide range of immunoassays, such as enzyme-linked immunoassays, such as those described in the examples below, sandwich immunoassays, homogeneous assays, immunohistochemistry formats, or other conventional assay formats. One of skill in the art may readily select from any number of conventional immunoassay formats to perform this invention.

Employing ligand binding to the biomarker proteins or multiple biomarkers forming the signature enables more precise quantitative assays, as illustrated by the ELISA assays.

Additionally, machine learning algorithms can be used in connection with this method. A variety of machine learning classifiers exist, wherein each classifier produces a unique machine learning process and/or output. The machine learning algorithms may comprise a biased algorithm or an unbiased algorithm. The biased algorithm may comprise Gene Set Enrichment Analysis (GSVA) enrichment of phenotype-associated cell-specific modules. The unbiased approach may employ all available phenotypic data. The machine learning algorithm may comprise a random forest (RF) classifier.

The random forest classifier is able to sort through the inherent heterogeneity of the plurality of records to identify one or more third records associated with the specific phenotype. In some embodiments, the classifier identifies said one or more third records associated with the specific phenotype with an accuracy of at least about 70%. The implementation of the random forest classifier herein enable a specific phenotype association sensitivity of 85% and a specific phenotype association specificity of 83%. Further classifier optimization, however, may yield improved results.

Alternatively, classification and regression tree analysis (CART) may be used in connection with the method. CART is a nonparametric decision tree methodology that has the ability to efficiently segment populations into meaningful subgroups. CART analysis classifies complex spectra and/or other information in order to distinguish subjects as normal or as having a particular medical condition. CART can be used to construct a decision rule and/or increase the speed and efficiency of the application of the decision rule and to avoid investigator bias. There are a variety of CART analyses that can provide useful results, including the C.50 program (Release 2.07 GPL Edition, available from rulequest.com), the M5P classifier, as implemented in Weka (available from weka.sourceforge.net/doc.stable/weka/classifiers/trees/M5P.html). There are other CART analyses available and this is not meant to be an exhaustive list.

In a preferred embodiment, in an ELISA assay performed, and at least 2 EP or SAB biomarkers are analyzed using a random forest classifier to predict EP and nonviable pregnancy. In another embodiment, at least 5 EP or SAB biomarkers are analyzed to predict EP and nonviable pregnancy. In another embodiment, an analysis may include using CART. In a preferred embodiment using CART, at least 3 EP or SAB biomarkers are analyzed to predict EP and nonviable pregnancy.

In one embodiment, suitable assays for use in these methods include immunoassays using antibodies or ligands to the above-identified biomarkers and biomarker signatures. In another embodiment, a suitable assay includes an ELISA assay for two more EP or SAB biomarkers that include one or more of the proteins/unique peptides in Tables 2A and 2B. The platform most likely to be used in clinical assays will be multiplexed or parallel sandwich ELISA assays or their equivalent, primarily because this platform is the technology most commonly used to quantify blood proteins in clinical laboratories.

### Nucleic Acid Assays

Still other methods useful in performing the diagnostic steps described herein are known in the art. Such methods include methods based on hybridization analysis of polynucleotides, methods based on sequencing of polynucleotides, proteomics-based methods or immunochemistry techniques. The most commonly used methods known in the art for the quantification of mRNA expression in a sample include northern blotting and in situ hybridization; RNAse protection assays; and PCR-based methods, such as reverse transcription polymerase chain reaction (RT-PCR) or qPCR. Alternatively, antibodies may be employed that can recognize specific DNA-protein duplexes. The methods described herein are not limited by the particular techniques selected to perform them. Exemplary commercial products for generation of reagents or performance of assays include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test), the MassARRAY-based method (Sequenom, Inc., San Diego, CA), differential display, amplified fragment length polymorphism (iAFLP), and BeadArray™ technology (Illumina, San Diego, CA) using the commercially available Luminex 100 LabMAP system and multiple color-coded microspheres (Luminex Corp., Austin, Tex.) and high coverage expression profiling (HiCEP) analysis.

Thus, in yet another embodiment, a method for diagnosing an ectopic pregnancy or a non-viable pregnancy in a female mammalian subject involves measuring in a biological fluid sample of the subject the expression level of a gene, gene fragment, gene transcript (e.g., mRNA) or expression product encoding one or more of the biomarkers. Alternatively, the method includes measuring the expression level of a gene, gene fragment, gene transcript or expression product encoding a combination of two or more biomarkers. The method further includes comparing the subject's selected biomarker gene, gene fragment, gene transcript or expression product expression level with the level of the same gene, gene fragment, gene transcript or expression product in the biological fluid of a reference or control female mammalian subject having a normal intrauterine pregnancy (IUP). Changes in expression of the subject's selected biomarker gene, gene fragment, gene transcript or expression products from those of the reference or control correlates with a diagnosis of ectopic pregnancy or non-viable pregnancy.

In another embodiment, the above method further includes measuring in the biological fluid sample of the subject the expression level of an additional biomarker gene, gene fragment, gene transcript or expression product encoding fragment of a biomarker. In another embodiment, the above method further includes measuring in the biological fluid sample of the subject the expression level of two or more additional biomarker gene, gene fragment, gene transcript or expression product encoding biomarkers.

In another embodiment, the above method further includes measuring in the biological fluid sample of the subject the expression level of an additional biomarker gene, gene fragment, gene transcript or expression product encoding fragment of a biomarker identified in Tables 2A and 2B.

In this diagnostic method, a change in expression level of one or more of the selected biomarker gene, gene fragment, gene transcript or expression product in comparison to the IUP control reference may be an upregulation or down regulation in the expression of the individual biomarker gene, gene fragment, transcript or expression product. This method may employ any of the suitable diagnostic reagents or kits or compositions described above.

In yet another embodiment, the methods and compositions described herein may be used in conjunction with clinical risk factors to help physicians make more accurate decisions about how to manage patients with ectopic pregnancies. Another advantage of these methods and compositions is that diagnosis may occur early.

The following examples are illustrative only and are not intended to limit the present invention.

### EXAMPLES

### Example 1: Validation of Biomarkers

### Materials and Methods

### Initial Study Design

Participants for the study consisted of 192 women with symptomatic (pain and/or bleeding) early pregnancy who presented for immediate evaluation to one of three academic centers (University of Pennsylvania, Eastern Virginia Medical school, Northwestern University) (65 IUPs, 66 EPs, and 61 SABs). Inclusion for the initial prospective cohort consisted of the following criteria: 1) complaints of abdominal pain, vaginal bleeding, or both; 2) serum hCG of 100-60,000 mIU/mL; 3) 5-12 weeks gestation by last menstrual period; and 4) agreement to participate in data and serum collection for the Ectopic Pregnancy Biomarkers Bank after informed consent. The hCG range inclusion criteria was purposefully expanded to evaluate if results would validate in a more pragmatic population. Institutional Review Board approval was obtained at all study sites. Participants were excluded if 1) they had received any treatment during the current pregnancy prior to enrollment; 2) they had evidence of gestational trophoblastic disease; 3) they were diagnosed with a non-tubal ectopic pregnancy; or 4) there was evidence of multiple gestation.

Specimens were collected from 2014-2017. The previously identified protocols for patient data collection, biomarker selection, serum collection, and sample assays were used herein. Briefly, maternal age, gestational age, race, ethnicity, study site, and initial hCG were collected for each subject upon entry and final pregnancy outcome was obtained through chart abstraction. Each participant was followed prospectively until their final pregnancy outcome, as defined by international consensus. A viable intrauterine pregnancy (IUP) was defined as ultrasound evidence of an intrauterine gestational sac, yolk sac, and fetal pole with cardiac activity. Spontaneous abortion (SAB) was categorized as an embryonic loss (fetal pole >4 mm with no cardiac activity) or an anembryonic gestation (gestational sac >16 mm with no identified yoke sac or fetal pole) or with no change in size of fetal pole of gestational sac one week apart with evidence of products of conception on histopathology. Ectopic pregnancy (EP) was defined as laparoscopic evidence or an extrauterine gestation or ultrasound demonstrating an adnexal mass without evidence of an intrauterine pregnancy or an increase in hCG level after uterine evacuation.

### Data Collection and Biomarker Assays

Serum was collected at the point of initial presentation, centrifuged at 1,500 rpm for 5 minutes, split into 0.5-mL aliquots, and stored at −80 C. Selected samples were sent to the University of Virginia Ligand Assay and Analysis Core and immunoassays performed. Based on both a putative and agnostic approach through prior investigations, the markers in the initial study included ADAM-12, Progesterone (P4), Activin A, PAPP-A, Glycodelin, and beta human chorionic gonadotropin (hCG). Assay characteristics are described in Table 1. All biomarkers were assessed on the same specimen for each subject.

TABLE 1
Biomarker Assay Characteristics

| | | | | Inter-assay |
|---|---|---|---|---|
| | | Functional | | Coefficient of |
| Biomarkers | Method/Vendor | Sensitivity | | Variation* |
| Placental-like growth | ELISA/R&D | 7.8 | pg/ml | 9.8% |
| factor (PIGF) | System | | | |
| A Disintegrin And | ELISA/R&D | 0.16 | ng/ml | 8.5% |
| Metalloprotease - 12 | System | | | |
| (ADAM12) | | | | |
| Progesterone | Immulite | 0.1 | ng/ml | 4.9% |
| | 2000/Siemens | | | |
| Activin A | ELISA/Ansh | 110 | pg/ml | 6.4% |
| Inhibin A | ELISA/Ansh | 15 | pg/ml | 5.7% |
| Pregnancy- | ELISA/R&D | 0.39 | ng/ml | 9.6% |
| associated plasma | System | | | |
| protein A (PAPP-A) | | | | |
| Pregnancy-specific | ELISA/Cusabio | 1.6 | ng/ml | 18.0% |
| beta 1-glycoprotein | | | | |
| (SP-1) | | | | |
| Glycodelin | ELISA/LS Bio | 4.7 | ng/ml | 10.8% |
| Vascular Endothelial | ELISA/R&D | 31 | pg/ml | 1.8% |

| | |
|---|---|
| Growth Factor | System |
| (VEGF) | |

*All intra-assay coefficients of variation <10%

### Sample Size

We assumed for the purposes of these calculations that our current markers have 95% or better accuracy among those classified (i.e. proportion of those classified that were true positives and true negatives). In order to obtain a two-sided 95% confidence interval for an observed accuracy rate of 95% will have a half-width of 3.1% (95% confidence interval 91.9% to 98.1%) we would need 63 cases and 120 controls.

Because EP was the rarest outcome, SAB and IUP subject selection was frequency balanced for initial hCG and gestational age to that of EP to provide the best representative samples. Based on the three clinical outcomes, we assigned case and control status based on the outcome of pregnancy location or viability. For the outcome of pregnancy location, EPs were classified as cases and IUPs and SABs were controls. For the outcome of pregnancy viability, EPs and SABs were classified as cases and IUPs were controls. Assays for each biomarker were run after study sample selection; thus selection was blind to biomarker profile.

### Statistical Analysis

Baseline characteristics of subjects were evaluated by using the Kruskal-Wallis test for continuous measures, and Pearson Chi-square or Fisher-exact tests for categorical variables. We first assessed the reproducibility of the assays for each marker individually. We evaluated the means, ranges, and standard deviations as well as area under receiver operating characteristic curves (AUCs) that determined discrimination for each maker stratified by outcome. The Kruskal-Wallis test was used to compare differences among biomarker distribution by pregnancy outcome and the Wilcoxon Rank Sum test used to assess biomarker differences by source population.

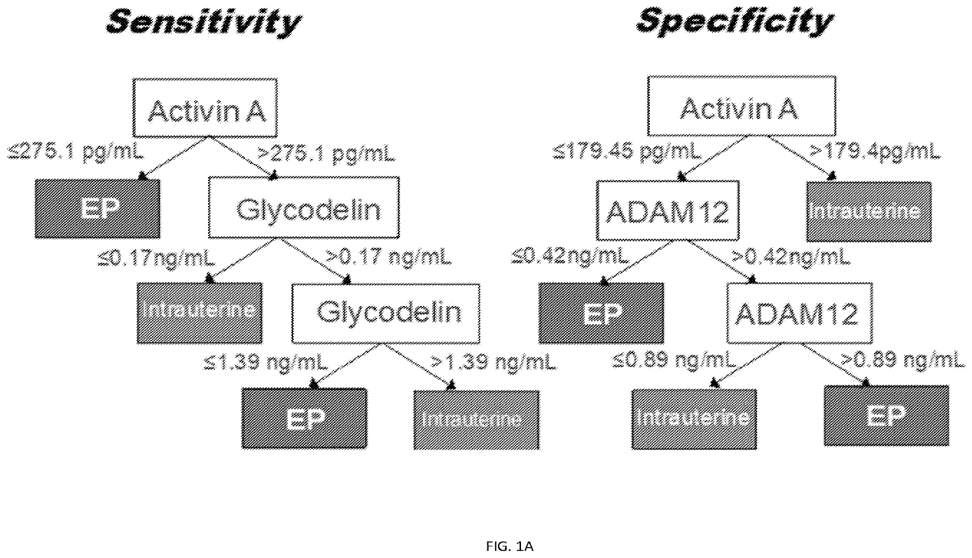

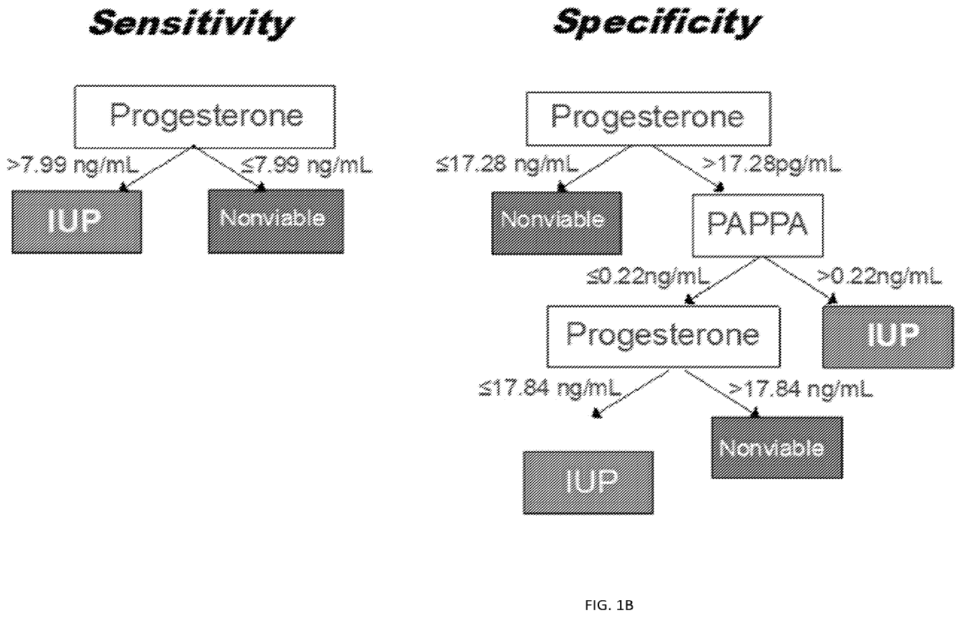

We next applied decision trees for using the markers simultaneously to predict a) location of the pregnancy (EP vs IUP+SAB) and b) viability of the pregnancy (IUP vs EP+SAB). Serum concentrations of each biomarker, for each subject, were used to create a prediction of outcome using the algorithm reported in our previous paper (FIG. 1). The predicted outcome was compared to actual outcome in order to assess the number of subjects definitively classified (and those who were inconclusive), as well as the accuracy of the prediction. Accuracy is a summary statistic defined as the proportion of true classifications out of all classifications (TN+TP)/(TN+TP+FP+FN). We assess prediction of each test (a) location and (b) viability, separately.

Validation of our model was assessed in a series of planned analyses determined if the model could be optimized. Analyses included 1) assessment of prediction based on the exact algorithm of biomarkers including the threshold for each marker derived from previous studies, 2) assessment of prediction of the model using the same biomarkers but allowing revision of the thresholds of the individual markers based on the concentrations found in the new population, and 3) assessment of prediction of the model if markers were added or removed markers for original model. We applied the decision trees to clinically important subgroups of patients including those less than six weeks gestational age and those with an hCG less than 2000 mIU/mL.

### Results

A total of 192 women composed of 65 IUPs, 66 EPs, and 61 SABs were included in the final analysis. Baseline characteristics are reported in Table 4. There were no differences between groups of participants in regards to race or ethnicity. Subjects with an IUP were more likely to be younger than those with an EP or SAB (p=0.0002). Women with IUPs presented with higher initial hCG values (p<0.0001). Women with EPs and SABs presented earlier than those with an IUP (p=0.0002).

TABLE 4
Baseline Characteristics of the study population

| | EP | IUP | SAB | |
|---|---|---|---|---|
| | (n = 66) | (n = 65) | (n = 61) | p-value |
| Maternal Age | 28 [24, 32] | 26 [21, 30] | 30 [27, 35] | 0.0002 |
| median (IQR) | | | | |
| Hispanic | 4 (6%) | 2 (3%) | 2 (3%) | 0.64 |
| Caucasian | 8 (12%) | 10 (15%) | 13 (21%) | 0.29 |
| Black | 55 (83%) | 47 (72%) | 42 (69%) | |
| Other | 3 (5%) | 8 (12%) | 6 (10%) | |
| hCG, | 2391 | 27991 | 8315 | <0.0001 |
| median (IQR) | [902, 6965] | [10816, 56149] | [4060, 18330] | |
| Gestational | 42 [37, 49] | 50 [43, 58] | 45 [42, 53] | 0.0002 |
| Age median | | | | |
| (IQR) | | | | |

Kruskal-Wallis test was used for continuous measures, and Pearson Chi-square or Fisher-exact tests were used for categorical variables.

### Assessing Reproducibility of Individual Biomarkers

The medians and ranges of all six markers produced for subject by outcome are presented in (Table 5) and compared to previously obtained values.

TABLE 5
Biomarker Distributions by Pregnancy Outcome and Source

| Biomarker | | EP | IUP | SAB | p-value⁺ | p-value* |
|---|---|---|---|---|---|---|
| ADAM12 | D | 0.43 [0.26-0.76] | 0.53 [0.36-0.82] | 0.59 [0.39-0.87] | 0.02 | 0.053 |
| | V | 0.42 [0.18-0.85] | 0.77 [0.43-1.47] | 0.88 [0.42-1.23] | <.001 | |
| Progesterone | D | 5.0 [2.9-13.5] | 21.0 [15.0-24.7] | 2.6 [1.5-6.4] | <.001 | 0.016 |
| | V | 4.6 [[2.4-12.21 | 19.6 [14.18-27.9] | 9.1 [5.17-14.4] | <.001 | |
| Activin A | D | 206.8 [161.4-254.1] | 289.3 [225.4-365.8] | 325.9 [265.6-460.3] | <.001 | 0.015 |
| | V | 231.1 [175.3-290.6] | 334.3 [255.9-514.8] | 402.5 [273.7-602.4] | <.001 | |
| PAPP-A | D | 0.18 [0.13-0.24] | 0.20 [0.15-0.27] | 0.26 [0.17-0.34] | <.001 | 0.819 |
| | V | 0.05 [0.00-0.09] | 0.55 [0.06-1.44] | 0.75 [0.29-1.67] | <.001 | |
| Glycodelin | D | 0.32 [0.22-0.60] | 0.2 [0.1- 0.41] | 0.26 [0.1-0.86] | 0.008 | 0.053 |
| | V | 0.00 [0.00-3.87] | 0.00 [0.00-12.88] | 0.00 [0.00-6.90] | 0.671 | |
| hCG | D | 823 [120-18733] | 4271 [124-19895] | 632 [102-17714] | <0.001 | <0.001 |
| | V | 2391 [902, 6965] | 27991 [10816, 56149] | 8315 [4060, 18330] | <0.001 | |

Values of presented in median [Interquartile Range]. D = Derivation study; V = Validation study.
⁺Kruskall-Wallis test for differences in median by outcome (EP vs. IUP vs. SAB).
*Wilcoxon Rank Sum test for differences in median by source (Validation vs. Derivation study)

Markers that were found to be statistically different across outcomes in the derivation population were also found to be statistically different across outcome in this new validation population. Exceptions include Glycodelin, in which levels were shown to have significantly different means among each pregnancy outcome in the derivation study, but values were similar in this validation population. We also evaluated biomarker differences between the derivation versus validation population (Table 5). The distribution of Progesterone, Activin A, and beta human chorionic gonadotropin were statistically different among the derivation and validation populations, although with overlapping interquartile ranges.

As a final assessment of reproducibility, we compared each biomarker's individual performance to determine discrimination among pregnancy outcomes by assessing area under the curve for the prediction of outcome for each marker individually (Table 6).

TABLE 6
Individual Biomarker Performance for Pregnancy Location and Viability

| | Pregnancy Location: | | Pregnancy Viability: | |
|---|---|---|---|---|
| | EP vs. IUP + SAB | | IUP vs. EP + SAB | |
| | Derivation Data | Validation Data | Derivation Data | Validation Data |
| Biomarker | AUC [95% CI] | AUC [95% CI] | AUC [95% CI] | AUC [95% CI] |
| Activin A | 0.79*⁺⁺ | 0.77*⁺⁺ | 0.56 | 0.58 |
| | [0.73-0.85] | [0.70, 0.83] | [0.49-0.64] | [0.49, 0.66] |
| PAPP-A | 0.61 | 0.80 | 0.55⁺⁺ | 0.61⁺⁺ |
| | [0.54-0.69] | [0.734, 0.87] | [0.47-0.62] | [0.52, 0.69] |
| Glycodelin | 0.61* | 0.54* | 0.60 | 0.53 |
| | [0.54-0.68] | [0.45, 0.62] | [0.52-0.68] | [0.43, 0.63] |
| ADAM12 | 0.60⁺⁺ | 0.67⁺⁺ | 0.51 | 0.59 |
| | [0.52-0.69] | [0.59, 0.75] | [0.43-0.58] | [0.51, 0.68] |
| Progesterone | 0.58 | 0.77 | 0.88*⁺⁺ | 0.84*⁺⁺ |
| | [0.50-0.65] | [0.69, 0.85] | [0.83-0.93] | [0.79, 0.90] |
| hCG | 0.61 | 0.80 | 0.76 | 0.81 |
| | [0.53-0.68] | [0.73, 0.87] | [0.70-0.83] | [0.74, 0.89] |

*Contribute to the sensitivity tree.
⁺⁺Contribute to the specificity tree

The biomarkers that contributed to the pregnancy location algorithm (Activin A, Glycodelin, ADAM12) and pregnancy viability algorithm (PAPP-A, Progesterone) all showed similar AUCs to the derivation study subject population.

### Assessing Number Classified and Accuracy of Prediction, Refining Algorithms, and Subgroup Comparisons

We then evaluated the number of pregnancies classified and accuracy of the decision algorithms using the original decision trees (FIG. 1). Pregnancy location was conclusively classified in 53% (n=94) of the whole study sample with 78% accuracy (compared to conclusively classification of 29% (n=67) with 100% accuracy in the derivation population). Pregnancy viability was conclusively classified in 58% (n=112) of the new sample with 89% accuracy (compared to conclusively classification of 61% (n=140) of patients with 97% accuracy) (Table 7). Results were similar in the model where the threshold for each biomarker was revised, and if we removed glycodelin and added hCG. The results were also similar in both the derivation and the validation sample in the subgroups of subject with a hCG below 2000 mIU/mL and a gestational age less than 6 weeks (Table 7).

TABLE 7
Comparison of Performance of Original Algorithm, Revisions, and Important Subgroups

| | | | | | Model Revision: Drop | |
|---|---|---|---|---|---|---|
| | Original | | Revision of Thresholds | | gylcodelin & add hCG | |
| | | Accuracy | | Accuracy | | Accuracy |
| | | Amongst | | Amongst | | Amongst |
| | Conclusive | Classified | Conclusive | Classified | Conclusive | Classified |
| | Classification | [95% CI] | Classification | [95% CI] | Classification | [95% CI] |
| Location | 67/228 | 100% | 67/228 | 100% | 75/228 | 99% |
| | (29%) | [95-100] | (29%) | [96-100] | (33%) | [93-100] |
| | 94/175 | 78% | 69/176 | 78% | 87/185 | 96% |
| | (54%) | [69-87] | (39%) | [67-87] | (47%) | [78-92] |
| Viability | 140/230 | 97% | 140/230 | 97% | 143/230 | 97% |
| | (61%) | [93-98] | (51%) | [93-88] | (62%) | [92-99] |
| | 112/192 | 89% | 112/192 | 89% | 108/175 | 87% |
| | (58%) | [81-94] | (58%) | [81-94] | (64%) | [79-93] |

| | | | Gestational Age <6 | | |
|---|---|---|---|---|---|
| | hGG <2000 | | Weeks | | |
| | | Accuracy | | Accuracy | |
| | | Amongst | | Amongst | |
| | Conclusive | Classified | Conclusive | Classified | |
| | Classification | [95% CI] | Classification | [95% CI] | |
| Location | 27/112 | 100% | 30/80 | 100% | Derivation |
| | 24% | [87-100] | 38% | [88-100] | |
| | 9/32 | 99% | 37/195 | 85% | Validation |
| | (28%) | [52-100] | (47%) | [76-92] | |
| Viability | 82/112 | 99% | 38/80 | 89% | Derivation |
| | 73% | [93-100] | 48% | [76-97] | |
| | 29/34 | 97% | 35/56 | 91% | Validation |
| | 85% | [82-100] | (63%) | [77-88] | |

### Discussion

The utility of a predictive test, or panel of tests will likely be greatly informed by its validity and accuracy. For a prediction model to be useful, it should be valid in a separate population from which it was derived. External validation includes both obtaining similar concentrations for each biomarker in a new population (assessing the heterogeneity of the predictor) as well as reproducing the test characteristics of a proposed companion diagnostic using a population distinct from its derivation. External validation is important for biomarker development. These data validate the possibly of prediction based on a model of multiplexed biomarkers in an external population from which they were derived.

Through iterative analysis, we have demonstrated that a possible companion diagnostic is best derived from multiple markers (preferably from diverse pathways). Because there are three outcomes (IUP, SAB and EP), it is preferable to dichotomize the outcome into two tests based on different biomarkers or thresholds (one for location and one for viability). Additionally, we have demonstrated that because the error of missing an EP or interruption of an IUP are both grave, it is optimal to develop a test that maximize accuracy of prediction, rather than maximizing sensitivity or specificity (at the expense of the other). If a woman received a prediction that is indeterminate, care can proceed using the current standard of serial hCG concentration and ultrasound.

Examination of our baseline characteristics of each pregnancy subgroup demonstrate that in general, women diagnosed with an IUP present with higher hCG, women with an EP present with a lower hCG, and women with a miscarriage present with an intermediate value. Additionally, those with EPs and SABs presented at earlier gestational ages. Given the typical manner in which women ultimately diagnosed with each of these entities present to care, these differences are expected and representative of clinical practice.

To assess if a biomarker reproduced similar values and prognostic results in a separate population, we assessed the mean values of each biomarker, in each outcome, in both the derivation and validation population. We also assessed the reproducibility of concentration of each biomarker (stratified by outcome) comparing mean values in the original and validation population. Finally, the ability of each biomarker to discriminate among pregnancy outcome was assessed with AUC. Our findings indicate that the concentration of most of biomarkers are reproducible in the separate external population. Exceptions included the median concentration of glycodelin, where there were statistically different outcomes in one, but not both, of the two populations (Table 5).

Assessment of the mean value of each marker (stratified by outcome) across populations was also similar with a few exceptions. The mean value of the biomarkers Progesterone, Activin A, and hCG were statistically different among the derivation and validation populations but may still be useful marker due to overlapping interquartile ranges in the two populations (Table 5). Assessment of each marker to discriminate outcome demonstrated that AUC for each biomarker (individually) was also very similar in both populations (Table 6). Based on these finding we concluded apart from glycodelin, the markers in our original multiple marker panel (Activin A, ADAM12, Progesterone and PAPP-A) demonstrated good initial individual validation as markers for pregnancy viability and location.

The next step of validation was the use of the markers in combination to assess the prediction of our models. External validation is the action of testing the original prediction model in a set of new patients to determine whether the model works to a satisfactory degree.

We noted that overall, the model validated well with a slight increase in the number of subjects classified and a slight decrease in accuracy of prediction. Prediction was not dramatically improved with revision of the thresholds of the cut points for markers to better reflect the actual values obtained in the new sample (Table 7).

We explored model revision because of the poor reproducibility of the results obtained with glycodelin. To assess if prediction could be improved, we removed glycodelin from our model and replaced it with hCG. The number of subjects classified and the accuracy of prediction was similar (Table 7). We applied the revised algorithms to clinically important subgroups including women with pregnancies <6 weeks gestation and pregnancies with hCG <2000 mIU/mL. Our revised algorithms performed with similar trends in test characteristics when compared to the whole study sample and in both populations.

While the model validated mathematically by obtaining similar results in a new population, it does not mean the model is clinically useful. Our approach and logic to this clinical conundrum was validated. The prediction of pregnancy location had definitive prediction in about 54% of the population with accuracy of 78-89%. The prediction of a nonviable gestation was modestly better, with definitive prediction in about 60% of subjects with accuracy ranging from 87-97%.

We propose herein that, in a pregnancy diagnostic test, the test characteristic that needs optimization is accuracy of prediction in order to minimize false positive and false negative prediction, resulting in the clinical consequences of missing an EP or false diagnosis of a gestation as nonviable. This approach resulted in high accuracy.

This data represents early development and validation of the use of biomarkers to aid in prediction of women at risk for a nonviable or ectopic pregnancy. Use of optimal biomarkers with well-phenotyped pregnancy outcomes, will allow for calculation of predictive value and specifically in women with more clinically challenging presentations such as a pregnancy of unknown location. Additionally, biomarkers could be assessed in combination with current diagnostic methods such as transvaginal ultrasound and perhaps serial assessment of hCG and progesterone.

In conclusion, these results demonstrate that the utilization of a panel of biomarkers using logic to maximize test accuracy of a prediction of pregnancy location as well as a prediction of pregnancy viability was reproducible and validated in an external population from which it was derived.

### Example 2: Identification and Evaluation of Optimal Biomarkers

### Materials and Methods

### Assessing Assay Performance

A list of all of our potential biomarker candidates using a combination of agnostic proteomic screening or hypothesized putative biological function is presented in Table 2A.

We screened ELISAs of each biomarker candidate in two pools of patient samples to discern functional sensitivity, assay reportable range, recovery/linearity, and intra-assay precision (% CV). Intra-assay precision was determined during the evaluation of each ELISA examined, and intra-assay % CVs were similar in assays used in previous studies (6,7). To control for assay-to-assay variability, we ran an equal number of samples from each group (IUP, EP, SAB) in each ELISA. Signal trend among the two sample pools was also investigated in exploratory analyses. One pool of samples was labeled the “low” pool, consisting of 30 individuals at risk of early pregnancy loss with human chorionic gonadotropin (hCG) values <1500 mIU/mL and gestational age (GA) four to six weeks, and the other pool of samples was labeled the “high” pool, consisting of 30 individuals with hCG values >4000 mIU/mL and GA seven to 11 weeks.

We analyzed cross-reactivity of each member of the pregnancy-specific family of glycoproteins (PSGs) using commercial reference preparations to ensure that each was specific to a single isoform and not cross-reactive with other highly homologous isoforms of the same protein in the ELISAs examined.

### Assessing Biomarker Assay Discrimination

After assessing assay performance, we then performed a case-control study of IUP, EP, and SAB specimens from our biobank to evaluate each candidate biomarker's ability to discriminate between location (EP vs. non-EP, Table 3A) and viability (IUP vs. EP and SAB, Table 3B).

TABLE 3A
Assay Discrimination: Location

| | | Mean | | | | 95% | | |
|---|---|---|---|---|---|---|---|---|
| | | (Standard | Median | | | Confidence | % Not | |
| Biomarker | | Deviation) | (Min-Max) | p-value | AUC | interval | Detected | Summary |
| PSG9 | EP | 1,333 (1435) | 873 (211-6,539) | <0.0001 | 0.871* | 0.799-0.943* | 0.00% | Good |
| | Non-EP | 5,439 (4,760) | 2856 (249-15,728) | | | | 0.00% | |
| PSG1 | EP | 17.64 (30.66) | 5.82 (1.6-118.9) | <0.0001 | 0.867* | 0.795-0.940* | 14.29% | Good |
| | Non-EP | 120.05 (126.5) | 79.66 (1.6-600) | | | | 2.86% | |
| IGFBP1 | EP | 6,634 (10,525) | 3,324.7 (630-51,730) | <0.0001 | 0.758* | 0.657-0.860* | 12.12% | Good |
| | Non-EP | 18,162 (21,555) | 8,920.5 (689-80,000) | | | | 0.00% | |
| KISS1⁺ | EP | 0.31 (0.34) | 0.16 (0.05-1.38) | 0.001 | 0.705* | 0.598-0.813* | 0.00% | Good |
| | Non-EP | 1.14 (3.24) | 0.33 (0.09-25) | | | | 0.00% | |
| PSG3 | EP | 2.14 (0.69) | 2.03 (1.3-4.67) | 0.0212 | 0.644* | 0.536-0.752* | 0.00% | Good |
| | Non-EP | 2.83 (1.66) | 2.37 (0.9-10.20) | | | | 1.43% | |
| PARVB | EP | 615.5 (481.65) | 442.5 (63-2,000) | 0.155 | 0.604 | 0.467-0.741 | 4.17% | Good |
| | Non-EP | 490.3 (466.01) | 343 (63-2000) | | | | 12.77% | |
| MMP9⁺ | EP | 345.8 (79.48) | 400 (123-400) | 0.251 | 0.552 | 0.448-0.656 | 0.00% | Moderate |
| | Non-EP | 345.9 (81.98) | 400 (70-400) | | | | 0.00% | |
| HE4⁺ | EP | 4,282 (1,223) | 3928 (2,714-7,558) | 0.457 | 0.545 | 0.426-0.664 | 0.00% | Moderate |
| | Non-EP | 4,159 (1,385) | 3,794 (2,552-10,000) | | | | 0.00% | |
| ISM2 | EP | 293.4 (248.06) | 219.09 (31-1,000) | 0.438 | 0.547 | 0.432-0.661 | 2.86% | Moderate |
| | Non-EP | 258.7 (229.13) | 190.01 (31-1,000) | | | | 11.43% | |
| PAEP | EP | 148.6 (136.72) | 102.27 (17.6-504.3) | 0.474 | 0.543 | 0.425-0.661 | 0.00% | Moderate |
| | Non-EP | 116.4 (95.26) | 97.28 (1.09-499.8) | | | | 1.41% | |
| CLIC1 | EP | 5.83 (4.86) | 4.31 (0.63-20) | 0.566 | 0.535 | 0.420-0.649 | 5.71% | Moderate |
| | Non-EP | 5.41 (4.69) | 3.82 (0.63-20) | | | | 10.14% | |
| MYLK | EP | 2.19 (2.15) | 1.51 (0.3-8) | 0.570 | 0.534 | 0.419-0.649 | 2.86% | Moderate |
| | Non-EP | 2.09 (2.05) | 1.32 (0.3-8) | | | | 5.71% | |
| QSOX2 | EP | 2.14 (2.1) | 0.25 (0.3-8) | 0.573 | 0.534 | 0.419-0.649 | 5.71% | Moderate |
| | Non-EP | 2.1 (2.15) | 1.26 (0.2-8) | | | | 5.71% | |
| NOTUM | EP | 1.71 (1.31) | 0.82 (0.8-5.85) | 0.064 | 0.613* | 0.498-0.729* | 48.39% | Poor |
| | Non-EP | 2.25 (1.43) | 2.11 (0.8-6.51) | | | | 32.84% | |
| WDR1 | EP | 27.34 (26.19) | 19.58 (3.1-100) | 0.529 | 0.538 | 0.423-0.653 | 8.57% | Poor |
| | Non-EP | 25.38 (24.87) | 18.36 (3.1-100) | | | | 11.43% | |
| EHD3⁺ | EP | 29.1 (25.4) | 21.76 (3.1-100) | 0.781 | 0.523 | 0.363-0.683 | 11.11% | Poor |
| | Non-EP | 30.8 (30.05) | 21.33 (3.1-100) | | | | 16.22% | |
| PLEK | EP | 444.8 (457.47) | 269.63 (62-2000) | 0.794 | 0.516 | 0.400-0.632 | 8.57% | Poor |
| | Non-EP | 416.8 (384.54) | 306.91 (62-1782) | | | | 16.18% | |
| ACTB⁺ | EP | 131.4 (57.02) | 115.67 (63.6-331.8) | 0.849 | 0.511 | 0.393-0.630 | 0.00% | Poor |
| | Non-EP | 125.9 (46.76) | 120.18 (53.4-301.2) | | | | 0.00% | |
| CD9 | EP | 0.99 (0.94) | 0.62 (0.6-5.06) | 0.006 | 0.659* | 0.554-0.763* | 75.76% | Poor |
| | Non-EP | 1.31 (0.96) | 0.99 (0.6-6.12) | | | | 43.55% | |
| LIMS1⁺ | EP | 0.106 (0.056) | 0.078 (0.08-0.29) | 0.062 | 0.596* | 0.504-0.687* | 77.14% | Poor |
| | Non-EP | 0.157 (0.191) | 0.078 (0.08-1.482) | | | | 58.57% | |
| ZYX | EP | 27.45 (18.28) | 23 (23-113.4) | 0.09 | 0.564* | 0.501-0.628* | 93.94% | Poor |
| | Non-EP | 38.34 (35.82) | 23 (23-154.8) | | | | 81.25% | |
| ITGB3 | EP | 139.6 (240.3) | 78 (78-1,421) | 0.77 | 0.512* | 0.434-0.589* | 85.29% | Poor |
| | Non-EP | 208.9 (664.0) | 78 (78-5,000) | | | | 82.54% | |
| ITGA2B | EP | 0.084 (0.023) | 0.078 (0.08-0.173) | 0.527 | 0.519* | 0.466-0.572* | 93.94% | Poor |
| | Non-EP | 0.112 (0.114) | 0.078 (0.08-0.586) | | | | 93.65% | |

*Denotes an inverted AUC (1- true AUC) so that all AUCs are >0.5 and comparable
⁺Denotes assay was run in singlet

TABLE 3B
Assay Discrimination: Viability

| | | Mean | | | | 95% | | |
|---|---|---|---|---|---|---|---|---|
| | | (Standard | Median | | | Confidence | % Not | |
| Biomarker | | Deviation) | (Min-Max) | p-value | AUC | interval | Detected | Summary |
| PSG9 | Viable | 6,736 (5,349) | 2,870 (616-15,728) | <0.0001 | 0.786 | 0.699-0.874 | 0.00% | Good |
| | Non-Viable | 2,738 (3,141) | 1,693 (211-15,548) | | | | 0.00% | |
| PSG3 | Viable | 3.03 (1.31) | 2.67 (1.4-6.94) | 0.032 | 0.731 | 0.628-0.834 | 0.00% | Good |
| | Non-Viable | 2.39 (1.47) | 1.98 (0.9-10.2) | | | | 1.43% | |
| EHD3 | Viable | 19.41 (23.43) | 11.38 (3.1-100) | 0.033 | 0.678* | 0.525-0.830* | 16.67% | Good |
| | Non-Viable | 35.51 (29.37) | 28.93 (3.1-100) | | | | 13.51% | |
| KISS1⁺ | Viable | 1.57 (4.39) | 0.33 (0.11-25) | 0.015 | 0.646 | 0.537-0.754 | 0.00% | Good |
| | Non-Viable | 0.51 (0.94) | 0.24 (0.05-7.40) | | | | 0.00% | |
| HE4⁺ | Viable | 3,827 (936) | 3,537 (2,731-6,792) | 0.026 | 0.634* | 0.519-0.749* | 0.00% | Good |
| | Non-Viable | 4,386 (1456) | 3,954 (2,552-10,000) | | | | 0.00% | |
| QSOX2 | Viable | 1.42 (1.41) | 1.05 (0.3-8) | 0.039 | 0.624* | 0.516-0.732* | 2.86% | Good |
| | Non-Viable | 2.46 (2.34) | 1.69 (0.3-8) | | | | 7.14% | |
| PSG1⁺ | Viable | 123.1 (153.3) | 77.73 (1.6-600) | 0.019 | 0.621 | 0.506-0.736 | 2.86% | Good |
| | Non-Viable | 67.34 (85.97) | 22.50 (1.6-308) | | | | 7.14% | |
| WDR1 | Viable | 19.2 (18.14) | 15.01 (3.1-100) | 0.119 | 0.594* | 0.484-0.704* | 8.57% | Moderate |
| | Non-Viable | 29.44 (27.57) | 21.45 (3.1-100) | | | | 11.43% | |
| MYLK | Viable | 1.55 (1.46) | 0.98 (0.3-8) | 0.145 | 0.588 | 0.302-0.523 | 2.86% | Moderate |
| | Non-Viable | 2.41 (2.27) | 1.57 (0.3-8) | | | | 5.71% | |
| PARVB | Viable | 470.2 (452.04) | 447.38 (63-2,000) | 0.337 | 0.570* | 0.425-0.715* | 12.50% | Moderate |
| | Non-Viable | 564.4 (483.05) | 374.62 (63-2,000) | | | | 8.51% | |
| PAEP | Viable | 102.4 (72.6) | 98.39 (1.09-267.2) | 0.41 | 0.549* | 0.435-0.663* | 2.78% | Moderate |
| | Non-Viable | 139.8 (124.95) | 98.05 (9.4-504.3) | | | | 0.00% | |
| IGFBP1 | Viable | 9,150 (9,452) | 6,119 (689-42,422) | 0.435 | 0.547* | 0.434-0.660* | 0.00% | Moderate |
| | Non-Viable | 17,206 (22,542) | 6,713 (630-80,000) | | | | 5.88% | |
| PLEK | Viable | 439.5 (379.33) | 379.84 (62-1782) | 0.532 | 0.538 | 0.417-0.659 | 11.76% | Poor |
| | Non-Viable | 419.8 (425.02) | 270.76 (62-2000) | | | | 14.49% | Poor |
| ACTB⁺ | Viable | 132.1 (56.11) | 123.21 (53.4-301.2) | 0.62 | 0.53 | 0.404-0.655 | 0.00% | Poor |
| | Non-Viable | 125.6 (47.25) | 114.54 (62.1-331.8) | | | | 0.00% | |
| ISM2 | Viable | 267.9 (216.62) | 210.39 (31-1,000) | 0.736 | 0.52 | 0.403-0.637 | 5.71% | Poor |
| | Non-Viable | 271.5 (245.16) | 180.63 (31-1,000) | | | | 10.00% | |
| MMP9⁺ | Viable | 351.3 (79.04) | 400 (147-400) | 0.688 | 0.517 | 0.412-0.622 | 0.00% | Poor |
| | Non-Viable | 347.6 (82.32) | 400 (70-400) | | | | 0.00% | |
| CLIC1 | Viable | 5.56 (4.45) | 4.63 (0.63-20) | 0.834 | 0.513 | 0.939-0.633 | 8.57% | Poor |
| | Non-Viable | 5.55 (4.85) | 3.77 (0.63-20) | | | | 8.70% | |
| CD9 | Viable | 1.46 (1.16) | 1.32 (0.6-6.12) | 0.032 | 0.627 | 0.511-0.742 | 37.93% | Poor |
| | Non-Viable | 1.09 (0.84) | 0.62 (0.6-5.06) | | | | 62.12% | |
| NOTUM | Viable | 2.32 (1.54) | 2.06 (0.8-6.51) | 0.288 | 0.564 | 0.446-0.683 | 30.30% | Poor |
| | Non-Viable | 1.96 (1.33) | 1.55 (0.8-5.85) | | | | 41.54% | |
| LIMS1⁺ | Viable | 0.109 (0.07) | 0.078 (0.08-0.335) | 0.112 | 0.581* | 0.488-0.675* | 74.29% | Poor |
| | Non-Viable | 0.156 (0.19) | 0.078 (0.08-1.482) | | | | 60.00% | |
| ITGA2B | Viable | 0.091 (0.06) | 0.078 (0.08-0.406) | 0.53 | 0.518* | 0.465-0.572* | 94.12% | Poor |
| | Non-Viable | 0.108 (0.109) | 0.078 (0.08-0.586) | | | | 90.63% | |
| ZYX | Viable | 34.03 (30.44) | 23 (23-142.5) | 0.931 | 0.503 | 0.428-0.579 | 84.85% | Poor |
| | Non-Viable | 34.94 (31.98) | 23 (23-154.8) | | | | 85.94% | |
| ITGB3 | Viable | 316.8 (895.4) | 78 (78-5,000) | 0.972 | 0.501 | 0.419-0.584 | 84.38% | Poor |
| | Non-Viable | 119.6 (178.52) | 78 (78-1,422) | | | | 83.08% | |

*Denotes an inverted AUC (1- true AUC) so that all AUCs are >0.5 and comparable
⁺Denotes assay was run in singlet

Each pregnancy type subgroup contained 70 samples from distinct patients for a total of 210 subjects. Serum biobank samples were collected from women with unassisted conceptions presenting for care due to risk of early pregnancy failure in the first trimester at both emergent and clinical care settings at the Hospital of the University of Pennsylvania, Northwestern University, or Eastern Virginia Medical School. Ultrasound was used to definitively determine the phenotype of the pregnancy (IUP, EP or SAB) at the time of serum collection or within 48 hours of serum collection (68).

Each subject had three aliquots of 510 μl serum each available for analysis. All samples were de-identified and stored at −80° C. By design and to further imply meaningful discrimination, we prioritized testing all of our potential biomarker candidates on the same individual. Due to limited volume for each individual subject and the volume needed to run each biomarker assay, each pregnancy subgroup of 70 subjects (total of 210) was divided into two groups of 35 subjects each (total of 105). Assays of candidate biomarkers with higher intra-assay CVs of >8% were ran in duplicate. Pleckstrin (PLEK), integrin subunit alpha IIb (ITGA2B), chloride intracellular channel 1 (CLIC1), activin B (ACTB), beta-parvin (PARVB), progestogen associated endometrial protein (PAEP), PSG1, KISS1, insulin-like growth factor binding protein 1 (IGFBP-1), matrix metallopeptidase 9 (MMP9), zyxin (ZYX), LIM and senescent cell antigen-like-containing domain protein 1 (LIMS1), and isthmin 2 (ISM2) were run on serum from one group of 105 subjects and palmitoleoyl-protein carboxylesterase NOTUM (NOTUM), EH domain-containing protein 3 (EHD3), WD repeat-domain containing protein 1 (WDR1), WAP four-disulfide core domain protein 2 (HE4), myosin light chain kinase (MYLK), PSG9, quiescin sulfhydryl oxidase 2 (QSOX2), integrin beta 3 (ITGB3), and CD9 antigen (CD9) were run on the other group of 105 subjects. Assay discrimination performance was assessed by Area Under the Curve (AUC) with 95% Confidence Intervals, measures of central tendency with two-sample t-tests comparing EP vs. non-EP and viable vs. non-viable, and visual plots.

### Results

Baseline characteristics of subjects with SABs, EPs, or IUPs are listed in Table 8. The majority of patients in all groups were Black and Non-Hispanic. As expected, based on the underlying clinical population, the mean hCG of patients with IUPs were higher than those with SABs and EPs and the mean gestational age of the SAB group was higher than either the EP or IUP group.

TABLE 8
Unassisted Pregnancy Subgroup Characteristics

| | SAB | EP | IUP |
|---|---|---|---|
| Race, n (%) | | | |
| Black | 32 (46) | 37 (53) | 51 (73) |
| White | 14 (20) | 15 (21) | 9 (13) |
| Other | 24 (34) | 16 (23) | 10 (14) |
| Unknown | 0 (0) | 2 (3) | 0 (0) |
| Ethnicity, n (%) | | | |
| Hispanic | 10 (14) | 9 (13) | 6 (9) |
| Non-Hispanic | 58 (83) | 58 (83) | 62 (89) |
| Unknown | 2 (3) | 3 (4) | 2 (3) |
| Mean hCG (mIU/mL) | 17, 161 | 5, 311 | 32, 439 |
| Mean GA based on last | 64 | 44 | 46 |
| menstrual period | (9 w 1 d) | (6 w 2 d) | (6 w 4 d) |
| days (weeks-days) | | | |
| Mean GA based on | 51 | NA | 44 |
| ultrasound | (7 w 2 d) | | (6 w 2 d) |
| days (weeks-days) | | | |

### Assessing Assay Performance

Biomarkers with assays that demonstrated at least acceptable recovery/linearity, moderate intra-assay precision in both low and high pools, and sufficient sensitivity were considered good or acceptable and selected for further study (Table 9).

TABLE 9
Assay Performance

| | | | | | Intra- | | | | |
|---|---|---|---|---|---|---|---|---|---|
| | | | | | assay | | | | |
| | Functional | | Assay | Recovery/ | Precision | “Low” | “High” | | |
| Biomarker | Sensitivity | | Range | Linearity | (% CV) | Pool | Pool | | Summary |
| PSG9 | 5 | ug/ml | 5-150 | Acceptable | 6.6% | 508 | 1461 | ug/ml | Good |
| | | | ug/ml | | | ug/ml | | | |
| PSG1⁺ | 1.6 | ng/ml | 1.6-200 | Good | 3.7% | 12.8 | 255.0 | ng/ml | Good |
| | | | ng/ml | | | ng/ml | | | |
| IGFBP1 | 63 | pg/ml | 63-8000 | Acceptable | 6.2% | 5,665 | 15,854 | pg/ml | Good |
| | | | pg/ml | | | pg/ml | | | |
| PSG3 | 0.31 | ng/ml | 0.31-50 | Acceptable | 6.7% | 1.35 | 6.0 | ng/ml | Good |
| | | | ng/ml | | | ng/ml | | | |
| PARVB | 63 | pg/ml | 63-2000 | Acceptable | 6.1% | 312 | 11,218 | ng/ml | Good |
| | | | pg/ml | | | pg/ml | | | |
| MMP9⁺ | 0.16 | ng/ml | 0.16-20 | Good | 4.3 | 518 | 618 | ng/ml | Good |
| | | | ng/ml | | | ng/ml | | | |
| HE4⁺ | 39 | pg/ml | 39-5000 | Acceptable | 5.8% | 4375 | 4349 | pg/ml | Acceptable |
| | | | pg/ml | | | pg/ml | | | |
| ISM2 | 31 | pg/ml | 31-1000 | OK | 6.1% | 215 | 244 | pg/ml | Acceptable |
| | | | pg/ml | | | pg/ml | | | |
| PAEP | 4.7 | ng/ml | 4.7-300 | Acceptable | 8.5% | 193.0 | 260.5 | ng/ml | Acceptable |
| | | | ng/ml | | | ng/ml | | | |
| CLIC1 | 0.63 | ng/ml | 0.63-20 | Acceptable | 6.2% | 4.9 | 5.3 | ng/ml | Acceptable |
| | | | ng/ml | | | ng/ml | | | |
| MYLK | 0.25 | ng/ml | 0.25-8.0 | OK | 6.5% | 0.28 | 1.02 | ng/ml | Acceptable |
| | | | ng/ml | | | ng/ml | | | |
| QSOX2 | 0.25 | ng/ml | 0.25-8.0 | OK | 7.4% | 2.0 | 1.7 | ng/ml | Acceptable |
| | | | ng/ml | | | ng/ml | | | |
| NOTUM | 0.8 | ng/ml | 0.8-100 | OK | 7.0% | 4.0 | 7.5 | ng/ml | Acceptable |
| | | | ng/ml | | | ng/ml | | | |
| WDR1 | 3.1 | ng/ml | 3.1-100 | OK | 6.9% | 22.6 | 23.7 | ng/ml | Acceptable |
| | | | ng/ml | | | ng/ml | | | |
| EHD3⁺ | 3.1 | ng/ml | 3.1-100 | Acceptable | 5.2% | 14.0 | 546 | ng/ml | Good |
| | | | ng/ml | | | ng/ml | | | |
| PLEK | 31 | ng/ml | 31-1000 | Acceptable | 9.2% | 127 | 4363 | ng/ml | Good |
| | | | ng/ml | | | ng/ml | | | |
| ACTB⁺ | 12 | pg/ml | 12-1400 | OK | 4.4% | 164.4 | 165.6 | ng/ml | Acceptable |
| | | | pg/ml | | | ng/ml | | | |
| CD9 | 0.31 | ng/ml | 0.31-20 | Acceptable | 9.9% | 2.5 | 3.5 | ng/ml | Acceptable |
| | | | ng/ml | | | ng/ml | | | |
| LIMS1⁺ | 0.078 | ng/ml | 0.078-10 | OK | 6.5% | 0.39 | 0.41 | ng/ml | Acceptable |
| | | | ng/ml | | | ng/ml | | | |
| ZYX | 23 | pg/ml | 47-1500 | OK | 9.3% | 169 | 174 | pg/ml | Acceptable |
| | | | pg/ml | | | pg/ml | | | |
| ITGB3 | 78 | pg/ml | 78-5000 | Acceptable | 8.7% | <78 | 6.18.9 | pg/ml | Acceptable |
| | | | pg/ml | | | pg/ml | | | |
| ITGA2B | 0.078 | ng/ml | 0.078-10 | OK | 6.4% | 0.27 | 0.51 | ng/ml | Acceptable |
| | | | ng/ml | | | ng/ml | | | |
| ILK | 0.78 | ng/ml | 0.78-50 | Unacceptable | 5.6% | 5.4 | 5.3 | ng/ml | Unacceptable |
| | | | ng/ml | | | ng/ml | | | |
| MUC9 | 0.15 | ng/ml | 0.15-20 | Acceptable | 5.8% | <0.15 | <0.15 | ng/ml | Unacceptable |
| | | | ng/ml | | | ng/ml | | | |

⁺Denotes assay was run in singlet

Trends in comparisons among the two hCG pools were assessed in exploratory analyses as a potential promising factor in evaluation of assay performance but were not used as a criterion for categorization. TAGLN2, MUC9, and ILK were rejected in assay performance analyses. The ELISAs for TAGLN2 and ILK were rejected due to poor analyte linearity (determined by serial dilution of commercial human serum pools). Although the performance of the MUC9 was acceptable analytically, the sensitivity was not sufficient.

Our analyses of the PSG family confirmed minimal cross-reactivity without evidence of systematic bias among the three assays for PSG1, PSG3 and PSG9.

### Assessing Biomarker Assay Discrimination

Biomarker candidates were ranked into three categories of “good”, “moderate”, or “poor” based on AUC, measures of central tendency (p value), and percentage of samples where the biomarker was not detected. “Good” candidates were considered those with an AUC >0.6 or with a significant p-value and where less than 20% of the samples had undetectable levels in both cases and controls. “Poor” markers were delineated from “Moderate” markers based primarily on comparing AUC and percentages of samples not being detected. Markers were automatically considered poor if greater than 20% of the sample had undetectable levels in either the cases or controls.

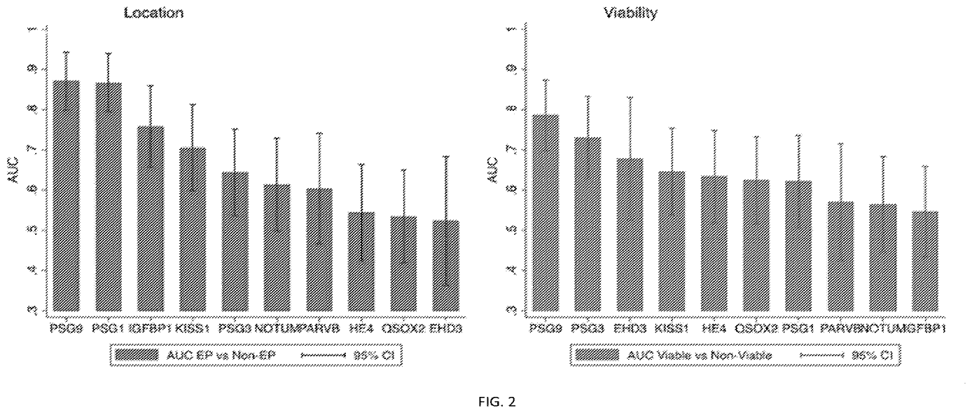

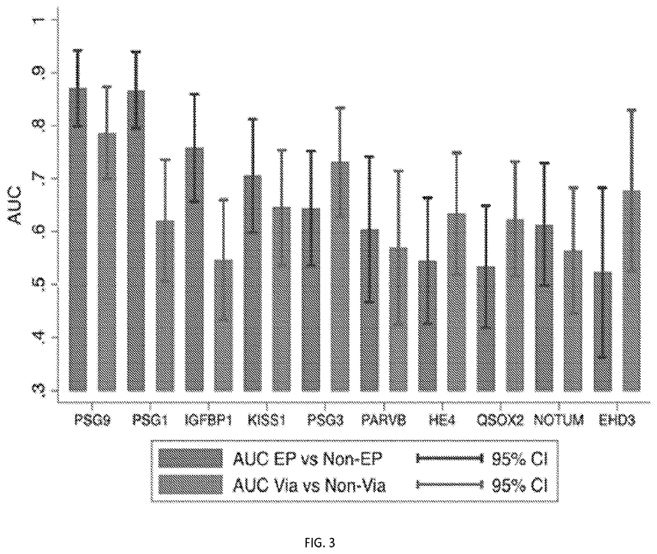

Markers were assessed for the ability to discriminate location of a pregnancy (EP vs IUP and SAB) (Table 3A) and separately for viability of a pregnancy (IUP vs SAB and EP) (Table 3B). The best biomarkers for discrimination of pregnancy location (EP vs. non-EP) were PSG9, PSG1, IGFBP1, KISS1, PSG3, and PARVB. The best biomarkers for discrimination of pregnancy viability (viable vs. non-viable) were PSG9, PSG3, EHD3, KISS1, HE4, QSOX2, and PSG1. AUCs of these best overlapping biomarkers for discrimination of location or viability are shown in FIG. 2. FIG. 3 shows the AUCs of each of these biomarkers for both location and viability.

### Discussion

Many biomarkers have been proposed for a wide range of clinical conditions, but most do not fulfill their promise upon validation. In the initial stage of biomarker development, the goal is to cast a wide net including not only markers with biological plausibility, but also those identified through an unbiased discovery platform, such as proteomics, as novel biomarkers may reflect currently unknown characteristics or properties of the clinical condition. Through careful iterative analysis we have successfully honed a large pool of novel candidates leading to six markers that may predict pregnancy location, seven markers that may predict viability, and four overlapping markers that may predict both location and viability.

Before applying assays to our three pregnancy subgroup populations, we first evaluated assay performance and concluded that the performance of three of our markers was unacceptable to use in our case control studies. Assessing assay performance of even those assays that are “commercially available” is critical prior to clinical implementation as not all function well. In the case of candidate biomarkers TAGLN2, MUC9, and ILK, reasons for assay rejection included poor linearity, poor recovery, and insufficient sensitivity.

After evaluating assay performance, we then assessed each biomarker's ability to discriminate among EP, IUP, and SABs. We found that PSG9, PSG1, IGFBP1, KISS1, PSG3, and PARVB were best able to discriminate pregnancy location. The PSG family of proteins has been well described with a proposed function relating to the early trophoblast, cell adhesion, and hemostasis. Due to similarities among members of this protein family, we assessed cross-reactivity in independent samples to ensure the candidacy of each of these members as a distinct biomarker. It is for this reason that earlier studies evaluating the role of PSG as a biomarker for abnormal pregnancy may be unreliable.

KISS1 and PARVB share overlapping biological functions with that of the PSG family with KISS1 demonstrating a role in both trophoblast function and cell adhesion and PARVB with a role in cell adhesion. Although previous studies have demonstrated differences in KISS1 expression among subgroups of early pregnancy (IUP, EP, pregnancy of unknown location [PUL]), previous work investigating the role of PARVB has been largely limited to discovery platform studies and animal experiments. Distinct from other markers in this group, IGFBP1 appears to have a role primarily related to the endometrium with previous investigations showing a role in both IUP and PUL subgroups.

PSG9, PSG3, EHD3, KISS1, HE4, QSOX2, and PSG1 were biomarker candidates best able to discriminate pregnancy viability. Although both EHD3 and QSOX2 share overlapping proposed roles pertaining to trophoblast function, scant literature exists beyond discovery proteomic experiments and basic science studies related to cell function. Indeed, evidence of any discriminatory ability among early pregnancy phenotypes is lacking. In the same vein, HE4, with a proposed role in glandular function at both the level of the endometrium and fallopian tube, lacks prior recognition as a possible biomarker for abnormal pregnancy.

Whether the pregnancy is a normally progressing IUP or an abnormal gestation in the uterus or fallopian tube, the initial process of pregnancy establishment may be similar with more marked changes as the gestation progresses. When considering SAB and EP, there may be even more overlap, further supporting that a two staged approach to utilizing biomarkers (location discrimination, viability discrimination) is most informative.

The strengths of this study include assessing a compilation of markers with both known biologic plausibility as well as novel candidates, expanding our present knowledge of the distinct physiologic differences among these subgroups of early pregnancy. Additionally, we used well-characterized assays and well-phenotyped specimens based on definitions from an international consensus group (68). Finally, testing each biomarker on the same subject, using the same laboratory, and performing analyses at the same time, automatically also controls for both known and unknown patient or laboratory characteristics, that in cases where different patient populations or multiple laboratory sites are used, might otherwise lead to possible discrepancies in results.

We have chosen modest criteria for good/poor results in order to move forward a larger pool of potential markers for evaluation and validation. With time and improvement in laboratory techniques, measures of performance and discrimination can be more rigid.

By improving detection of ectopic pregnancy, biomarkers have the potential to facilitate noninvasive intervention, reduce morbidity, and avoid detrimental intervention from misdiagnosis. We have demonstrated six biomarkers that noninvasively distinguish the location of an early pregnancy, seven markers that distinguish viability of an early pregnancy, and four overlapping markers that maybe distinguish both.

### Example 4: Validation of Additional Biomarkers

### Materials and Methods

### Participants

Participants for the study were selected from an ongoing, prospective cohort. Institutional Review Board approval was obtained prior to sample collection. Serum biobank samples were collected from women with unassisted conceptions presenting for care due to risk of early pregnancy failure in the first trimester at both emergent and clinical care settings at the Hospital of the University of Pennsylvania, Northwestern University, or Eastern Virginia Medical School. Ultrasound was used to definitively determine the phenotype of the pregnancy (IUP, EP or SAB) at the time of serum collection or within 48 hours of serum collection. This nested case-control design consisted of 218 women with symptomatic (pain and/or bleeding) early pregnancy (75 IUPs, 68 EPs, and 75 SABs). Each subject had three aliquots of 510 μl serum each available for analysis. All samples were de-identified and stored at −80 degrees Celsius.

Inclusion for the initial prospective cohort consisted of the following criteria: 1) complaints of abdominal pain, vaginal bleeding, or both; 2) serum hCG of 100-60,000 mIU/mL; 3) 5-10 weeks gestation by last menstrual period; and 4) agreement to participate in data and serum collection for the Ectopic Pregnancy Biomarkers Bank after informed consent. Specimens were selected from the bank from those collected to be representative of each three outcomes (not to be similar across outcomes). Participants were excluded if 1) they had received any treatment during the current pregnancy prior to enrollment; 2) they had evidence of gestational trophoblastic disease; 3) they were diagnosed with a non-tubal ectopic pregnancy; or 4) there was evidence of multiple gestation.

### Analysis of and Biomarker

Selected samples were sent to the University of Virginia Ligand Assay and Analysis Core and immunoassays performed. All biomarkers were assessed on the same specimen for each subject. The biomarker and assays for each are listed in table 10 (table of markers)

TABLE 10
Additional Candidate Biomarkers

| | | | Intra-Assay |
|---|---|---|---|
| Markers | Full name | Biologic plausibility | % CVs |
| PSG1 | Pregnancy-specific beta- | Trophoblast function, cell | 3.7% |
| | 1-glycoprotein 1 | adhesion, hemostasis | |
| sFLT | Soluble fms-like | Abnormal angiogenesis, | 4.1% |
| | tyrosine kinase-1 | tyrosine kinase | |
| GDF15 | Growth-differentiation | Trophoblast and decidual | 3.3% |
| | faction-15, macrophage | cell function, cell | |
| | inhibitory cytokine- 1 | signaling, cell | |
| | | development, Regulates | |
| | | food intake, energy | |
| | | expenditure and body | |
| | | weight in response to | |
| | | metabolic and toxin- | |
| | | induced stresses, Highly | |
| | | expressed in placenta | |
| PSG9 | Pregnancy-specific beta- | Trophoblast function, cell | 6.6% |
| | 1-glycoprotein 9 | adhesion, hemostasis | |
| hCG | Human chorionic | Trophoblast function | 4.9% |
| | gonadotropin | (total hcg alpha and beta) | |
| CG-ALPHA | | Trophoblast function, | 3.5% |
| | | glycoprotein hormones | |
| | | subunit alpha, Shared | |
| | | alpha chain of the active | |
| | | heterodimeric | |
| | | glycoprotein hormones | |
| | | thyrotropin/thyroid | |
| | | stimulating hormone/TSH, | |
| | | lutropin/luteinizing | |
| | | hormone/LH, | |
| | | follitropin/follicle | |
| | | stimulating hormone/FSH | |
| | | and | |
| | | choriogonadotropin/CG | |
| Adam 12 | A disintegrin and | Trophoblast function, | 4.5% |
| | metalloprotease-12 | metalloproteinase, cell | |
| | | adhesion | |
| SIGLEC-6 | Sialic acid-binding Ig- | Carbohydrate binding, | 5.1% |
| | like lectin 6 | lectin, expressed highly | |
| | | in placenta | |
| ANGPT2 | Angiopoietin 2 | In the absence of | 7.2% |
| | | angiogenic inducers, such | |
| | | as VEGF, ANGPT2- | |
| | | mediated loosening of | |
| | | cell-matrix contacts may | |
| | | induce endothelial cell | |
| | | apoptosis with consequent | |
| | | vascular regression. In | |
| | | concert with VEGF, it | |
| | | may facilitate endothelial | |
| | | cell migration and | |
| | | proliferation, thus serving | |
| | | as a permissive | |
| | | angiogenic signal. | |
| PSG3 | Pregnancy-specific beta- | Trophoblast function, cell | 6.7% |
| | 1-glycoprotein 3 | adhesion, hemostasis | |
| TFPI2 | Tissue factor pathway | Trophoblast function, cell | 6.8% |
| | inhibitor 2 | structure, blood | |
| | | coagulation, extracellular | |
| | | matrix structural | |
| | | constituent | |
| ActivinA | Activin A | Cell adhesion and | 4.2% |
| | | regulation | |
| PLGF | Placental growth factor | Developmental protein, | 5.3% |
| | | Cell signaling, | |
| | | angiogenesis, | |
| | | differentiation | |
| PAPPA | Pregnancy-associated | ABL trophoblast function, | 2.3% |
| | plasma protein-A | metalloproteinase and cell | |
| | | regulation | |
| PRG | Progesterone | Corpus luteum function | 4.3% |
| EHD3 | EH domain-containing | Cell structure regulation, | 5.2% |
| | protein 3 | trophoblast function, | |
| | | protein transport | |
| HAGH | | Metallo-β-Lactamase | 7.8% |
| ELAFIN | Elafin, elastase-specific | Proteinase inhibitor | 5.3% |
| | inhibitor | | |
| FIBRONECTIN | Fibronectin | Cell adhesion and | 6.2% |
| | | heparin-binding | |
| KISSPEPTIN | Metastasis-suppressor of | Trophoblast invasion and | 4.5% |
| | KISS-1; Kisspeptin-1 | migration, cell adhesion | |
| OPN | Osteopontin | Acts as a cytokine | 6.7% |
| | | involved in enhancing | |
| | | production of interferon- | |
| | | gamma and interleukin-12 | |
| | | and reducing production | |
| | | of interleukin-10 and is | |
| | | essential in the pathway | |
| | | that leads to type I | |
| | | immunity. | |
| CD9 | CD9 antigen, | Cell adhesion and | 9.9% |
| | tetraspanin protein | regulation, sperm-egg | |
| | family | fusion, hemostasis | |
| IGFBP1 | Insulin-like growth | Carrier protein, | 6.2% |
| | | endometrial function, | |
| | factor binding protein 1 | implantation, cell | |
| | | migration | |
| NOTUM | Palmitoleoyl-protein | Trophoblast function, cell | 7.0% |
| | carboxylesterase | signaling (Wnt signaling | |
| | NOTUM | pathway) | |

### Statistical Analysis

Baseline characteristics of subjects were evaluated by using the Kruskal-Wallis test for continuous measures, and Pearson Chi-square or Fisher-exact tests for categorical variables. For analysis of biomarkers, the concentration of each maker was compared and subgroups were combined to assess each marker's ability to predict viability (IUP vs SAB+EP) and Location (EP vs SAB+IUP) in separate analyses. The markers with the strongest association singly, were evaluated in companion assess prediction of each outcome.

### CART Analysis

Classification and regression tree analysis (CART) was performed Minitab (CARTPro v 6.0, Salford Systems). Trees were created that balanced sensitivity and specificity using 5-fold cross validation and set no cost. Separate trees that maximize sensitivity were also created. Additionally, specify when cost of error was analyzed when cost was set as high as possible. Multiple trees were created, including one with all biomarkers and one confined to markers highest individual prediction. Additional trees were created by removing the dominating biomarker from the subsequent tree (repeat this process 4 times for a total of 4 additional trees). Adjustments were made to the maximized sensitivity and specificity trees for both viability and location in order to further improve sensitivity/specificity and/or use only top variables for the given outcome.

The best of the optimizes trees for sensitivity and specificity were used in conjunction for a two-step prediction to minimize accuracy. If the same outcome (normal or abnormal) was achieved with both the tree for specificity and the tree for sensitivity, the outcome was classified as normal or abnormal. If the two trees predicted different outcomes, the prediction was considered indeterminate and a classification was not made. This process was repeated for the prediction of viability and location.

The predicted classification by each model was compared to actual diagnosis and sensitivity, specificity, positive predictive value (PPV), negative predictive value (NPV), conclusive classification, and accuracy amongst classified calculated.

Finally, the test for viability and location were used serially. The accuracy of this combined approach was assess with 95% CI obtained with Bootstrapping. The possible outcomes are listed below:
If viability test reports normal, then conclude normal. If viability test reports abnormal, then conclude abnormal.
If viability test reports inconclusive and location test reports inconclusive, then conclude inconclusive.
If viability test reports inconclusive and location test reports EP, then conclude abnormal.
If viability test reports inconclusive and location test reports non-EP, then conclude (abnormal or inconclusive).

### Random Forest

We assessed the predictive ability of biomarker with SVM-Polynominal, SVM-Linear, SVM-RBF, Logistic-Rge, Logistic regression with lasso, Logistic regression with elasticnet, Random forest and DMN. Iterative analysis evaluated three class prediction as well as classification as viability (IUP vs SAB+EP) and Location (EP vs SAB+IUP). Data used included values of all biomarker (10 for viability and 11 for location). Nested ten-fold cross validation was used to tune parameter and evaluate models. Model were created to balance sensitivity and specificity as well as alter cost function to maximize each with a limit of accuracy >0.08. Results of prediction were obtained for each model and using the optimized model for sensitivity and the optimal model for specificity in a two-step process to optimize prediction.

While all methods produced similar results and had similar feature selection, the best performed regarding AUC and accuracy was with the use of Random Forest. We explored the RF model to assess a parsimonious model with limited number of makers based on biological plausibility and agonistic selection using a limited number of markers.

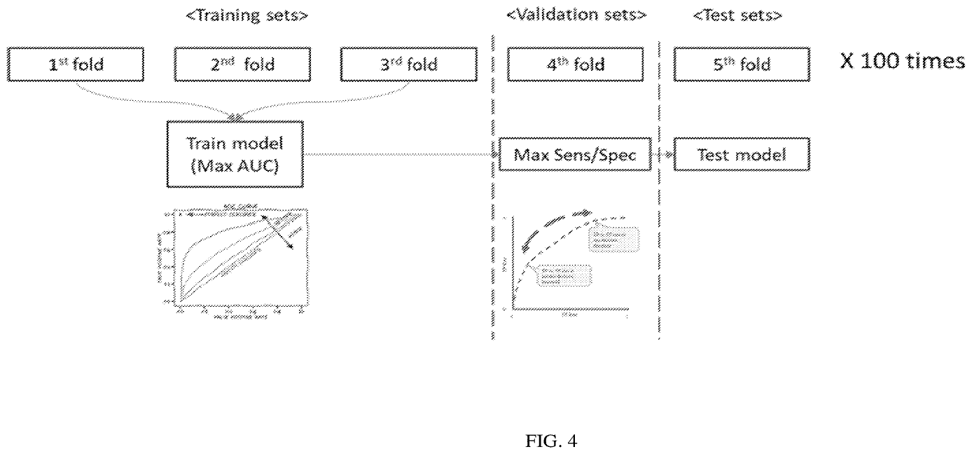

A graphical representation of the Random Forest methodology is provided as FIG. 4.

### Results

Demographic information of the subjects with SABs, Eps, or IUPs are provided in Table 11.

TABLE 11
Baseline Demographics

| | SAB | EP | IUP |
|---|---|---|---|
| | (n = 75) | (n = 68) | (n = 75) |
| Race | | | |
| Asian | 3 (4.0) | 3 (4.4) | 4 (5.3) |
| Black | 51 (68.0) | 42 (61.8) | 56 (74.7) |
| White | 15 (20.0) | 18 (26.5) | 10 (13.3) |
| Unknown | 6 (8.0) | 5 (7.4) | 5 (6.7) |
| Ethnicity | | | |
| Hispanic | 5 (6.7) | 6 (8.8) | 3 (4.0) |
| Non-Hispanic | 69 (92.0) | 60 (88.2) | 69 (92.0) |
| Unknown | 1 (1.3) | 2 (2.9) | 3 (4.0) |
| Mean hCG (mIU/mL) | 12,909.0 | 4,032.0 | 40,413 |
| | (5,019.0- | (1,365.7- | (18,524.1) |
| | 22,024.0) | 7,518.0) | |
| Mean GA based on | 68.5 (55.0-82.0) | 45.5 (18.2) | 46.9 (6.9) |
| last menstrual | | | |
| period (days) | | | |
| Mean GA based | 48.0 (44.0-56.0) | 41.4 (4.2) | 45.4 (4.4) |
| on ultrasound | | | |
| (days) | | | |

Data are presented as n (%) for categorical variables and median (IQR) or mean (SD) for continuous variables.

The results from the pregnancy location assay (EP vs. IUP and SAB) are provided in Table 12 as the concentration of biomarker for each outcome.

TABLE 12
Biomarker Performance: Location

| | Out- | | | Std | | | | |
|---|---|---|---|---|---|---|---|---|
| Biomarker | come | N | Mean | Dev | Median | Min | Max | AUC |
| Activin | EP | 68 | 0.375 | 0.16 | 0.339 | 0.14 | 0.90 | 0.34 |
| A | Non | 150 | 0.542 | 0.35 | 0.436 | 0.13 | 2.28 | |
| | EP | | | | | | | |
| ADAM12 | EP | 68 | 0.592 | 0.474 | 0.465 | 0.156 | 2.646 | 0.261 |
| | Non | 150 | 1.113 | 1.028 | 0.849 | 0.156 | 10.015 | |
| | EP | | | | | | | |
| ANGPT2 | EP | 68 | 3732.36 | 2282.5 | 2949.73 | 470 | 13897.5 | 0.266 |
| | Non | 150 | 6453.91 | 5107.12 | 4874.02 | 1683.45 | 30000 | |
| | EP | | | | | | | |
| PRG | EP | 67 | 8.758 | 8.773 | 7.2 | 0.741 | 49.9 | 0.382 |
| | Non | 150 | 10.267 | 6.138 | 9.6 | 0.1 | 33.3 | |
| | EP | | | | | | | |
| sFLT | EP | 68 | 849.64 | 784.77 | 515.48 | 289.54 | 4235.5 | 0.138 |
| | Non | 148 | 2543.52 | 2122.71 | 2046.41 | 288.41 | 16869.94 | |
| | EP | | | | | | | |
| PSG1 | EP | 68 | 16.071 | 24.301 | 3.428 | 3.2 | 114.61 | 0.11 |
| | Non | 150 | 90.902 | 75.949 | 70.144 | 3.2 | 381.58 | |
| | EP | | | | | | | |
| PSG3 | EP | 68 | 12.239 | 8.025 | 9.255 | 0.625 | 33.902 | 0.272 |
| | Non | 150 | 43.067 | 72.639 | 21.637 | 0.96 | 457.994 | |
| | EP | | | | | | | |
| PSG9 | EP | 68 | 4030.07 | 6347.54 | 1783.03 | 85.26 | 32363.75 | 0.168 |
| | Non | 150 | 17072.37 | 14743.95 | 13368.53 | 495.18 | 87952.3 | |
| | EP | | | | | | | |
| NOTUM | EP | 68 | 0.942 | 0.596 | 0.78 | 0.78 | 4.27 | 0.467 |
| | Non | 150 | 0.991 | 0.616 | 0.78 | 0.78 | 5.098 | |
| | EP | | | | | | | |
| PAPPA | EP | 68 | 0.428 | 0.182 | 0.39 | 0.39 | 1.711 | 0.363 |
| | Non | 150 | 0.871 | 1.927 | 0.39 | 0.39 | 20.644 | |
| | EP | | | | | | | |
| PLGF | EP | 68 | 12.698 | 4.806 | 11.179 | 7.777 | 35.93 | 0.352 |
| | Non | 149 | 16.509 | 13.546 | 14.067 | 7.777 | 149.12 | |
| | EP | | | | | | | |
| EHD3 | EP | 68 | 19.96 | 16.141 | 14.92 | 3.1 | 75.92 | 0.618 |
| | Non | 150 | 15.468 | 18.475 | 7.954 | 3.1 | 100 | |
| | EP | | | | | | | |
| Fibronectin | EP | 68 | 210489.6 | 72785.4 | 202249.3 | 16658.2 | 522893.1 | 0.396 |
| | Non | 150 | 232308.4 | 61763.2 | 222376.5 | 108324.6 | 438861.4 | |
| | EP | | | | | | | |
| hCG | EP | 68 | 7639.3 | 12022.5 | 3011 | 339 | 60645 | 0.17 |
| | Non | 150 | 29534.4 | 22600.3 | 26167.5 | 269 | 112645 | |
| | EP | | | | | | | |
| Kisspeptin | EP | 68 | 4.137 | 5.233 | 2.335 | 0.556 | 25 | 0.413 |
| | Non | 150 | 6.563 | 7.705 | 2.842 | 0.02 | 25 | |
| | EP | | | | | | | |
| IGFBP1 | EP | 68 | 13889.4 | 15286.9 | 7398.2 | 630 | 71692.9 | 0.432 |
| | Non | 150 | 17560.7 | 20041 | 11197.3 | 1115.8 | 80000 | |
| | EP | | | | | | | |
| GDF15 | EP | 68 | 1351.35 | 1260.83 | 1003.23 | 117 | 6873.09 | 0.163 |
| | Non | 150 | 4012.43 | 2862.23 | 3403.65 | 447.76 | 15000 | |
| | EP | | | | | | | |
| HAGH | EP | 68 | 12.805 | 4.531 | 12.298 | 5.977 | 30.2 | 0.384 |
| | Non | 150 | 14.413 | 4.59 | 13.257 | 1.203 | 31.997 | |
| | EP | | | | | | | |
| CD9 | EP | 68 | 0.963 | 1.845 | 0.31 | 0.31 | 13.623 | 0.43 |
| | Non | 144 | 1.416 | 2.718 | 0.36 | 0.31 | 20 | |
| | EP | | | | | | | |
| TFPI1 | EP | 67 | 127.6 | 88.79 | 102.01 | 39 | 652.99 | 0.276 |
| | Non | 149 | 328.24 | 365.36 | 207.53 | 41.236 | 1855.4 | |
| | EP | | | | | | | |
| SIGLEC-6 | EP | 67 | 2533.36 | 5811.1 | 326.49 | 237 | 29328.91 | 0.262 |
| | Non | 150 | 2626.72 | 4586.84 | 1139.8 | 237 | 30000 | |
| | EP | | | | | | | |
| OPN | EP | 67 | 10.92 | 9.01 | 8.964 | 2.434 | 60 | 0.578 |
| | Non | 149 | 8.85 | 6.79 | 7.057 | 1.692 | 60 | |
| | EP | | | | | | | |
| ELAFIN | EP | 68 | 8646.91 | 10107.25 | 5033.15 | 747.8 | 40000 | 0.385 |
| | Non | 150 | 10606.02 | 10590.5 | 6527.31 | 368.7 | 40000 | |
| | EP | | | | | | | |
| CG- | EP | 68 | 2498.86 | 3799.67 | 973.01 | 500 | 19338.34 | 0.192 |
| ALPHA | Non | 150 | 9572.63 | 8663.87 | 7656.9 | 500 | 48954.23 | |
| | EP | | | | | | | |

| | | | | T- | | | |
|---|---|---|---|---|---|---|---|
| | | | AUC | test | | % | |
| | Out- | 95% | (Same | p- | Ranksum | Not | |
| Biomarker | come | CI | Direction) | value | p-value | Detected | Direction |
| Activin | EP | 0.269-0.420 | 0.655 | 0.0003 | 0.0002 | 0.00% | Down |
| A | Non | | | | | 0.00% | in |
| | EP | | | | | | EP |
| ADAM12 | EP | 0.189-0.333 | 0.739 | 0.0001 | <0.0001 | 4.41% | Down |
| | Non | | | | | 2.67% | in |
| | EP | | | | | | EP |
| ANGPT2 | EP | 0.194-0.338 | 0.734 | <0.0001 | <0.0001 | 1.47% | Down |
| | Non | | | | | 1.33% | in |
| | EP | | | | | | EP |
| PRG | EP | 0.302-0.462 | 0.618 | 0.147 | 0.005 | 0.00% | Down |
| | Non | | | | | 0.67% | in |
| | EP | | | | | | EP |
| sFLT | EP | 0.081-0.194 | 0.862 | <0.0001 | <0.0001 | 0.00% | Down |
| | Non | | | | | 0.00% | in |
| | EP | | | | | | EP |
| PSG1 | EP | 0.064-0.157 | 0.89 | <0.0001 | <0.0001 | 50.00% | Down |
| | Non | | | | | 3.33% | in |
| | EP | | | | | | EP |
| PSG3 | EP | 0.206-0.337 | 0.728 | <0.0001 | <0.0001 | 0.00% | Down |
| | Non | | | | | 0.00% | in |
| | EP | | | | | | EP |
| PSG9 | EP | 0.111-0.225 | 0.832 | <0.0001 | <0.0001 | 0.00% | Down |
| | Non | | | | | 0.00% | in |
| | EP | | | | | | EP |
| NOTUM | EP | 0.420-0.513 | 0.533 | 0.582 | 0.199 | 91.18% | Down |
| | Non | | | | | 83.33% | in |
| | EP | | | | | | EP |
| PAPPA | EP | 0.308-0.417 | 0.637 | 0.061 | 0.0001 | 91.18% | Down |
| | Non | | | | | 56.67% | in |
| | EP | | | | | | EP |
| PLGF | EP | 0.272-0.431 | 0.648 | 0.025 | 0.0005 | 5.88% | Down |
| | Non | | | | | 3.36% | in |
| | EP | | | | | | EP |
| EHD3 | EP | 0.536-0.700 | 0.618 | 0.086 | 0.005 | 4.41% | Up |
| | Non | | | | | 12.00% | in |
| | EP | | | | | | EP |
| Fibronectin | EP | 0.312-0.480 | 0.604 | 0.023 | 0.014 | 0.00% | Down |
| | Non | | | | | 0.00% | in |
| | EP | | | | | | EP |
| hCG | EP | 0.112-0.229 | 0.83 | <0.0001 | <0.0001 | 0.00% | Down |
| | Non | | | | | 0.00% | in |
| | EP | | | | | | EP |
| Kisspeptin | EP | 0.334-0.492 | 0.587 | 0.019 | 0.04 | 2.94% | Down |
| | Non | | | | | 12.00% | in |
| | EP | | | | | | EP |
| IGFBP1 | EP | 0.348-0.516 | 0.568 | 0.181 | 0.109 | 4.41% | Down |
| | Non | | | | | 5.33% | in |
| | EP | | | | | | EP |
| GDF15 | EP | 0.106-0.219 | 0.837 | <0.0001 | <0.0001 | 1.47% | Down |
| | Non | | | | | 0.67% | in |
| | EP | | | | | | EP |
| HAGH | EP | 0.302-0.466 | 0.616 | 0.017 | 0.006 | 0.00% | Down |
| | Non | | | | | 1.33% | in |
| | EP | | | | | | EP |
| CD9 | EP | 0.353-0.506 | 0.57 | 0.214 | 0.08 | 55.88% | Down |
| | Non | | | | | 45.14% | in |
| | EP | | | | | | EP |
| TFPI1 | EP | 0.209-0.343 | 0.724 | <0.0001 | <0.0001 | 2.99% | Down |
| | Non | | | | | 0.00% | in |
| | EP | | | | | | EP |
| SIGLEC-6 | EP | 0.176-0.347 | 0.738 | 0.899 | <0.0001 | 32.84% | Down |
| | Non | | | | | 4.67% | in |
| | EP | | | | | | EP |
| OPN | EP | 0.497-0.660 | 0.578 | 0.064 | 0.066 | 1.49% | Up |
| | Non | | | | | 0.67% | in |
| | EP | | | | | | EP |
| ELAFIN | EP | 0.299-0.471 | 0.615 | 0.201 | 0.007 | 7.35% | Down |
| | Non | | | | | 8.00% | in |
| | EP | | | | | | EP |
| CG- | EP | 0.131-0.252 | 0.808 | <0.0001 | <0.0001 | 2.99% | Down |
| ALPHA | Non | | | | | 0.00% | in |
| | EP | | | | | | EP |

Eleven markers were noted to have an AUC or prediction of >0.7 and a P value of <0.001 for prediction of viability or location (or both) (PSG1, sFLT, GDF15 PSG9, hCG, CG-Alpha, Adam12, ANGPT2, TFPI2, PSG3, PRG. (Table 13).

TABLE 13
Ranking of Biomarkers

| EP | | | Viability | | |
|---|---|---|---|---|---|
| | AUC | | | AUC | |
| AUC | Direction | Ttest p | AUC | Direction | Ttest p |

Sorted by EP

| | | | | | | |
|---|---|---|---|---|---|---|
| PSG1 | 0.110 | 0.890 | <0.001 | 0.656 | 0.656 | 0.034 |
| sFLT | 0.138 | 0.862 | <0.001 | 0.710 | 0.710 | 0.006 |
| GDF15 | 0.163 | 0.837 | <0.001 | 0.830 | 0.830 | <0.001 |
| PSG9 | 0.168 | 0.832 | <0.001 | 0.868 | 0.868 | <0.001 |
| hCG | 0.170 | 0.830 | <0.001 | 0.850 | 0.850 | <0.001 |
| CG-ALPHA | 0.192 | 0.808 | <0.001 | 0.865 | 0.865 | <0.001 |
| Adam12 | 0.261 | 0.739 | <0.001 | 0.474 | 0.526 | 0.222 |
| SIGLEC-6 | 0.262 | 0.738 | 0.899 | 0.629 | 0.629 | 0.453 |
| ANGPT2 | 0.266 | 0.734 | <0.001 | 0.491 | 0.509 | 0.886 |
| PSG3 | 0.272 | 0.728 | 0.001 | 0.902 | 0.902 | <0.001 |
| TFPI2 | 0.276 | 0.724 | <0.001 | 0.448 | 0.552 | 0.291 |
| Activin A | 0.345 | 0.655 | <0.001 | 0.394 | 0.606 | 0.020 |
| PLGF | 0.352 | 0.648 | 0.025 | 0.498 | 0.502 | 0.669 |
| PAPPA | 0.363 | 0.637 | 0.060 | 0.385 | 0.615 | 0.996 |
| PRG | 0.382 | 0.618 | 0.147 | 0.817 | 0.817 | <0.001 |
| EHD3 | 0.618 | 0.618 | 0.085 | 0.392 | 0.608 | 0.088 |
| HAGH | 0.384 | 0.616 | 0.017 | 0.648 | 0.648 | 0.002 |
| ELAFIN | 0.385 | 0.615 | 0.201 | 0.554 | 0.554 | 0.455 |
| Fibronectin | 0.396 | 0.604 | 0.023 | 0.555 | 0.555 | 0.125 |
| Kisspeptin | 0.413 | 0.587 | 0.019 | 0.544 | 0.544 | 0.248 |
| OPN | 0.578 | 0.578 | 0.064 | 0.357 | 0.643 | 0.004 |
| CD9 | 0.430 | 0.570 | 0.214 | 0.485 | 0.515 | 0.848 |
| IGFBP1 | 0.432 | 0.568 | 0.181 | 0.330 | 0.670 | <0.001 |
| NOTUM | 0.467 | 0.533 | 0.582 | 0.543 | 0.543 | 0.694 |

Sorted by Viability

| | | | | | | |
|---|---|---|---|---|---|---|
| PSG3 | 0.272 | 0.728 | 0.001 | 0.902 | 0.902 | <0.001 |
| PSG9 | 0.168 | 0.832 | <0.001 | 0.868 | 0.868 | <0.001 |
| CG-ALPHA | 0.192 | 0.808 | <0.001 | 0.865 | 0.865 | <0.001 |
| hCG | 0.170 | 0.830 | <0.001 | 0.850 | 0.850 | <0.001 |
| GDF15 | 0.163 | 0.837 | <0.001 | 0.830 | 0.830 | <0.001 |
| PRG | 0.382 | 0.618 | 0.147 | 0.817 | 0.817 | <0.001 |
| sFLT | 0.138 | 0.862 | <0.001 | 0.710 | 0.710 | 0.006 |
| IGFBP1 | 0.432 | 0.568 | 0.181 | 0.330 | 0.670 | <0.001 |
| PSG1 | 0.110 | 0.890 | <0.001 | 0.656 | 0.656 | 0.034 |
| HAGH | 0.384 | 0.616 | 0.017 | 0.648 | 0.648 | 0.002 |
| OPN | 0.578 | 0.578 | 0.064 | 0.357 | 0.643 | 0.004 |
| SIGLEC-6 | 0.262 | 0.738 | 0.899 | 0.629 | 0.629 | 0.453 |
| PAPPA | 0.363 | 0.637 | 0.060 | 0.385 | 0.615 | 0.996 |
| EHD3 | 0.618 | 0.618 | 0.085 | 0.392 | 0.608 | 0.088 |
| Activin A | 0.345 | 0.655 | <0.001 | 0.394 | 0.606 | 0.020 |
| Fibronectin | 0.396 | 0.604 | 0.023 | 0.555 | 0.555 | 0.125 |
| ELAFIN | 0.385 | 0.615 | 0.201 | 0.554 | 0.554 | 0.455 |
| TFPI2 | 0.276 | 0.724 | <0.001 | 0.448 | 0.552 | 0.291 |
| Kisspeptin | 0.413 | 0.587 | 0.019 | 0.544 | 0.544 | 0.248 |
| NOTUM | 0.467 | 0.533 | 0.582 | 0.543 | 0.543 | 0.694 |
| Adam12 | 0.261 | 0.739 | <0.001 | 0.474 | 0.526 | 0.222 |
| CD9 | 0.430 | 0.570 | 0.214 | 0.485 | 0.515 | 0.848 |
| ANGPT2 | 0.266 | 0.734 | <0.001 | 0.491 | 0.509 | 0.886 |
| PLGF | 0.352 | 0.648 | 0.025 | 0.498 | 0.502 | 0.669 |

### Classification Regression Tree

Feature selection using CART demonstrated PSG3, PSG9, CG_ALPHA, hCG, GDF15, and PRG were most predictive of viability and PSG1, sFLT, GDF15, PSG9, hCG, and CG_ALPHA were most predictive of location. The best models to predict viability used one maker (PSG3). The maximum sensitivity archived by a model was 93.3%. The maximum specificity archived was 98.6. Specificity refers to the number of women correctly classified as not have an ectopic pregnancy/the number of women actually not having ectopic pregnancies. Sensitivity refers to the number of women correctly classified as having an ectopic pregnancy/the number of women actually having ectopic pregnancies. PPV refers to the number of women correctly classified as having an ectopic pregnancy/the number of women that test positive (ectopic pregnancy); in other words, accuracy for ectopic pregnancies and NPV refers to the number of women correctly classified as not having an ectopic pregnancy/the number of women that test negative (non-ectopic pregnancy); in other words, accuracy for non-ectopic pregnancies. The model with the highest accuracy was 97.4% (with 70.2% receiving conclusive classification). (Table 14A)

TABLE 14A
Performance Model to predict location (EP vs SAP + IUP)

| | | | | | | Accuracy |
|---|---|---|---|---|---|---|
| Outcome | | | | | Conclusive | Amongst |
| (Protein Level) | Sensitivity | Specificity | PPV | NPV | Classification | Classified |
| Max Spec | 43/68 | 143/150 | 43/50 | 143/168 | 218/218 | 186/218 |
| Option 1(sFLT | (63.2%) | (95.3%) | (86.0%) | (85.1%) | (100%) | (85.3%) |
| Max Sens | 67/68 | 92/150 | 67/125 | 92/93 | 218/218 | 159/218 |
| Option 1 (PSG3 | (98.5%) | (61.3%) | (53.6%) | (98.9%) | (100%) | (72.9%) |
| and | | | | | | |
| TFPI2 | | | | | | |
| Balanced | 56/68 | 126/150 | 56/80 | 126/138 | 218/218 | 182/218 |
| Option 1 (PSG1 | (82.4%) | (84.0%) | (70.0%) | (91.3%) | (100%) | (83.5%) |
| Cross of max | 43/68 | 92/150 | 43/50 | 92/93 | 143/218 | 135/143 |
| Spec and Sens | (63.2%) | (61.3%) | (86.0%) | (98.9%) | (65.6%) | (94.4%) |
| to optimize | | | | | | |
| accuracy | | | | | | |
| (sFLT | | | | | | |
| PSG3 | | | | | | |
| and TFPI2) | | | | | | |

The best models to predict location used three markers (sFLT, PSG3 and TFPI2). The maximum sensitivity archived by a model was 98.5%. The maximum specificity archived was 95.3. The model with the highest accuracy was 94.4% (with 65.6% receiving conclusive classification). (Table 14B)

TABLE 14B
Performance of Model to predict Viability

| Outcome | | | | | | Accuracy |
|---|---|---|---|---|---|---|
| (Protein | | | | | Conclusive | Amongst |
| Levels) | Sensitivity | Specificity | PPV | NPV | Classification | Classified |
| Max Spec | 43/75 | 140/143 | 43/46 | 140/172 | 218/218 | 183/218 |
| Option 1 | (57.3%) | (97.9%) | (93.5%) | (81.4%) | (100%) | (83.9%) |
| (PSG3 (29.57), | | | | | | |
| PAPPA (0.52)) | | | | | | |
| Max Spec | 43/75 | 141/143 | 43/45 | 141/173 | 218/218 | 184/218 |
| Option 2 (CGA | (57.3%) | (98.6%) | (95.6%) | (81.5%) | (100%) | (84.4%) |
| (10960) and | | | | | | |
| PAPPA | | | | | | |
| (0.585)) | | | | | | |
| Max Sens | 70/75 | 109/143 | 70/104 | 109/114 | 218/218 | 179/218 |
| Option 1 | (93.3%) | (76.2%) | (67.3%) | (95.6%) | (100%) | (82.1%) |
| (PSG3 (16.7)) | | | | | | |
| Balanced | 70/75 | 109/143 | 70/104 | 109/114 | 218/218 | 179/218 |
| Option 1 | (93.3%) | (76.2%) | (67.3%) | (95.6%) | (100%) | (82.1%) |
| (PSG3 (16.7)) | | | | | | |
| Cross | 43/75 | 109/143 | 43/46 | 109/114 | 160/218 | 152/160 |
| Max Spec | (57.3%) | (76.2%) | (93.5%) | (95.6%) | (73.4%) | (95.0%) |
| Option 1 & | | | | | | |
| Max Sens | | | | | | |
| Option 1 | | | | | | |
| (PSG3 (29.57), | | | | | | |
| PAPPA (0.52) | | | | | | |
| Cross | 41/75 | 108/143 | 41/42 | 108/111 | 153/218 | 149/153 |
| Max Spec | (54.7%) | (75.5%) | (97.6%) | (97.3%) | (70.2%) | (97.4%) |
| Option 2 & | | | | | | |
| Max Sens | | | | | | |
| Option 1 | | | | | | |
| (CGA (10960) | | | | | | |
| and PAPPA | | | | | | |
| (0.585) and | | | | | | |
| PSG3 (29.57) | | | | | | |

When the models were used serially (viably used first and when inconclusive the results of the location tests were evaluated), the accuracy was 95.9% with 72.7% receiving conclusive classification). (Table 15)

TABLE 15
The MEANS Procedure

| | | | | Lower 95% | Upper 95% |
|---|---|---|---|---|---|
| Variable | N | Mean | Std Dev | CL for Mean | CL for Mean |
| sensitivity | 1000 | 54.49 | 4.22 | 54.23 | 54.76 |
| specificity | 1000 | 78.65 | 4.37 | 78.38 | 78.93 |
| ppv | 1000 | 99.20 | 1.04 | 99.14 | 99.26 |
| npv | 1000 | 90.37 | 3.27 | 90.17 | 90.57 |
| percent_classified | 1000 | 67.19 | 3.14 | 67.00 | 67.39 |
| accuracy | 1000 | 94.80 | 1.81 | 94.69 | 94.91 |

### Random Forest

All 11 markers were used in combination using other artificial intelligence models. While all methods produced similar results and had similar feature selection, the best performed regarding AUC and accuracy was with the use of Random Forest.

To predict viability, the maximum sensitivity archived by a model was 99%. The maximum specificity archived was 95%. The model with the highest accuracy was 97% (with 65% receiving conclusive classification). (Table 16A).

TABLE 16A
Performance of Random Forest (RF) Model to Predict Viability (IUP vs. SAB + EP)

| | | | | % | Accuracy Amongst |
|---|---|---|---|---|---|
| Test | Markers | Sensitivity | Specificity | Classified | Classified |
| Max Sens. | All 10 | 99% ± 3% | 39% ± 19% | 100% | 79% ± 6% |
| Max Spec. | All 10 | 75% ± 11% | 95% ± 11% | 100% | 81% ± 6% |
| Balanced | All 10 | 91% ± 6% | 83% ± 12% | 100% | 88% ± 6% |
| Cross of | All 10 | | | 65% | 97% |
| Max Sens. | | | | | |
| and | | | | | |
| Max Spec. | | | | | |
| 6 Markers | PSG3, PSG9, | 98% | 91% | 69% | 94% |
| | CG_ALPHA, | | | | |
| | hCG, PRG, | | | | |
| | CGF15 | | | | |
| 2 Markers | PSG3, PSG9 | 92% | 95% | 73% | 91% |

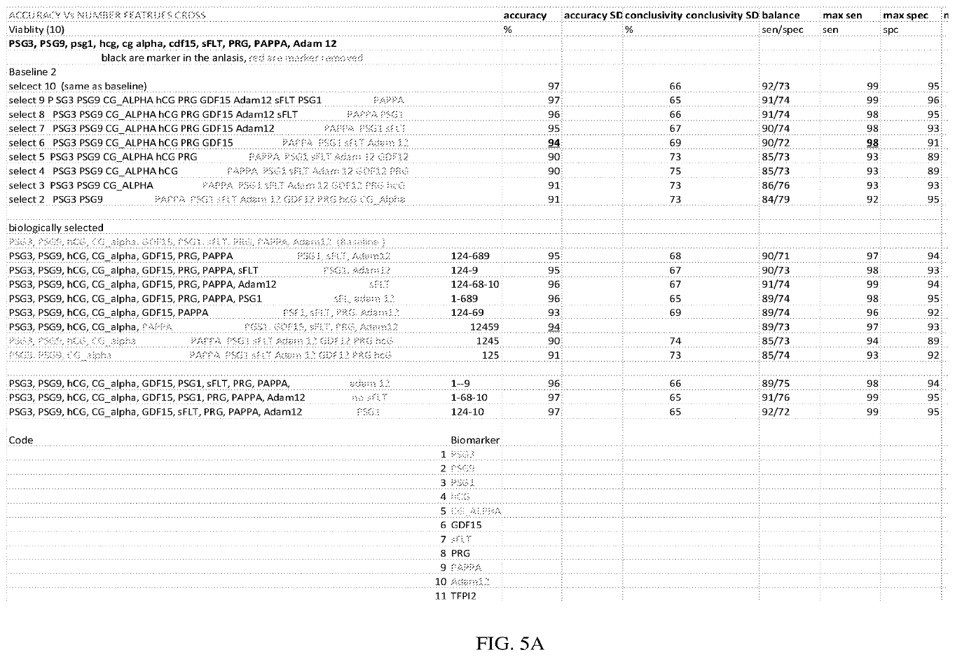

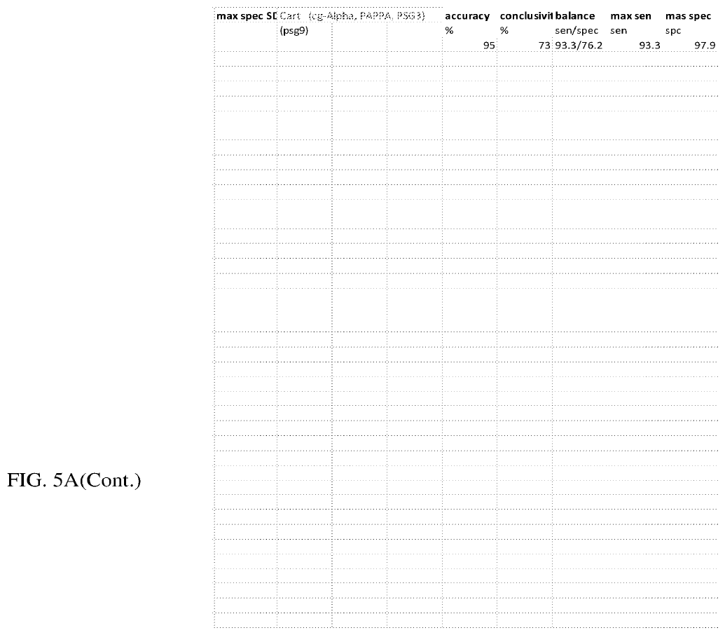

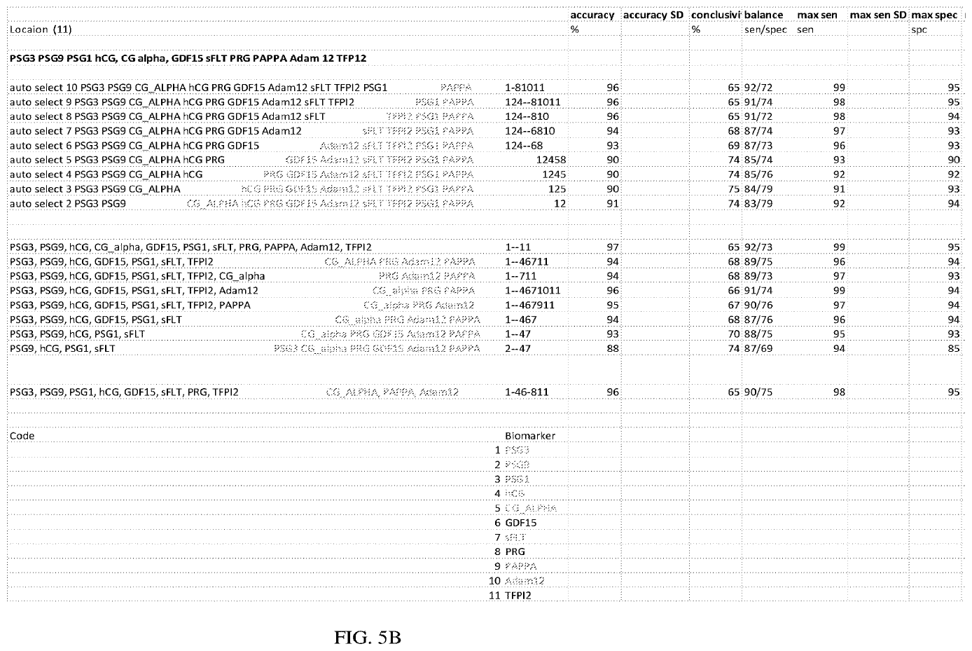

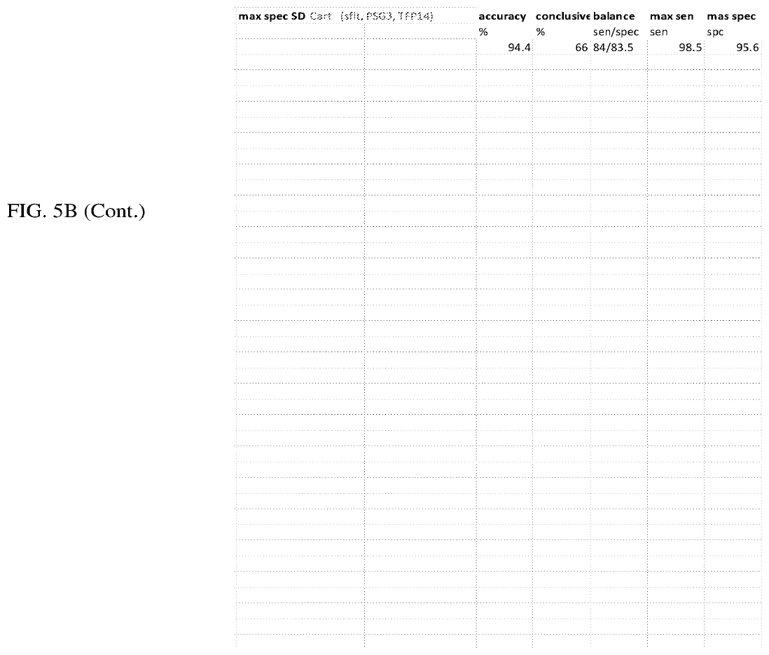

If the accuracy model was reduced to 6 markers the accuracy was 94% (with 69% receiving classification). If only two markers were used in the maximum accuracy models the accuracy was 91% (73% classified) (FIG. 5A).

To predict viability maximum sensitivity archived by a model was 99%. The maximum specificity archived was 95%. The model with the highest accuracy was 96% % (with 65% receiving conclusive classification). (Table 16B)

TABLE 16B
Performance of Random Forest (RF) Model to Predict Location (EP vs. SAB + IUP)

| | | | | | | | |
|---|---|---|---|---|---|---|---|
| | | | | | | | Accuracy |
| | | | | | | % | Amongst |
| Test | Markers | Sensitivity | Specificity | PPV | NPV | Classified | Classified |

| | | | | | |
|---|---|---|---|---|---|
| Max | All 11 | 99% ± 3% | 39% ± | 100% | 79% ± 6% |
| Sens. | | | 19% | | |
| Max | All 11 | 75% ± | 95% ± | 100% | 81% ± 6% |
| Spec. | | 11% | 11% | | |
| Balanced | All 11 | 92% ± | 72% ± | 100% | 86% ± 8% |
| | | 11% | 21% | | |
| Cross of | All 11 | | | 65% | 96% |
| Max | | | | | |
| Sens. and | | | | | |
| Max | | | | | |
| Spec. | | | | | |
| 6 | PSG3, | 96% | 93% | 69% | 93% |
| Markers | PSG9, | | | | |
| | CG_ALPHA, | | | | |
| | hCG, PRG, | | | | |
| | CGF15 | | | | |
| 2 | PSG3, PSG9 | 92% | 94% | 74% | 91% |
| Markers | | | | | |

If the accuracy model was reduced to 6 markers the accuracy was 93% (with 69% receiving classification). If only two markers were used in the maximum accuracy models the accuracy was 91% (74% classified) (FIG. 5B)

### Discussion

We have narrowed a large number pool of potential markers that can predict the viability or lection of a pregnancy (or both). Exploration of various AI modeling has demonstrated that accurate prediction can be obtain using CART or RF. Depending on the number of markers used prediction varies modestly.

We have also demonstrated the option of a novel model to maximize accuracy. The model can be adjusted to maximize the desired test characteristics. Such decisions will have advantages and limitations. Maximize accuracy will limit false neg and false positives but will limit classification to less the 100% of all subjects.

The data indicates that the best approach is to maximize accuracy (and thus minimize both false positive and false negative). Those who do not receive a classification can be followed with standard of care.

CART is the leading AI model, but we explored other AI methods. RF consistently produced the best results. RF modestly more accurate but CART used fewer markers and more intuitive. RF was explored to use a reduced number of features and there was a models decline in accuracy as features removed.

The data presented herein demonstrates that a relatively small pool of markers can be used to classify women with potential early pregnancy loss as viable and extra uterine. With the values of these makers, one can choose a model that maximizes sensitively, specify or accuracy. The overall test characteristics are modestly dependent on the number of markers used in the model but can be as few as 3 for each test.

All publications cited in this specification are incorporated herein by reference. While the invention has been described with reference to particular embodiments, it will be appreciated that modifications can be made without departing from the spirit of the invention.

Such modifications are intended to fall within the scope of the appended claims.

### REFERENCES FROM EXAMPLES 1-4

1. KISS1-Metastasis-suppressor KiSS-1 precursor-Homo sapiens (Human)-KISS1 gene & protein. 2018. Available at: https://www.uniprot.org/uniprot/Q15726. Accessed Jun. 1, 2022.
2. Larsson A, Palm M, Basu S, Axelsson, O. Insulin-like growth factor binding protein-1 (IGFBP-1) during normal pregnancy. 2012. Francis & Taylor Online. Available at: https://www.tandfonline.com/doi/full/10.3109/09513590.2012.730574?scroll-top&need Access=true. Accessed Jun. 1, 2022.
3 Bonassi S, Neri M, Puntoni R. Validation of biomarkers as early predictors of disease. Mutation Research 2001; 480 (1): 349-58.
4. Hall J A, Brown R, Paul J. An exploration into study design for biomarker identification: Issues and recommendations. Cancer Genomics Proteomics 2007; 4:111-20
5. Rothman N, Stewart W F, Schulte P A. Incorporating biomarkers into cancer epidemiology: A matrix of biomarker and study design categories. Cancer Epidem Biomark Prevention 1995; 4:301-11.
6. Rausch M E, Sammel M D, Takacs P, Chung K, Shaunik A, Barnhart K T. Development of a multiple marker test for ectopic pregnancy. Obstet Gynecol 2011; 117:573-82.
7. Senapati S, Sammel M D, Butts S F, Takacs P, Chung K, Barnhart K T. Predicting first trimester pregnancy outcome: derivation of a multiple marker test. Fertil Steril. 2016; 106:1725-32.
8. Beer L A, Tang, H Y, Sriswadi S, Barnhart K T, Speicher D W. Systematic discovery of ectopic pregnancy serum biomarkers using 3-D protein profiling coupled with label-free quantitation. J Proteome Res 2011; 10:1126-38.
9. Sullivan-Pyke C, Haisenleder D J, Senapati S, Nicolais O, Eisenberg E, Sammel M D, et al. Kisspeptin as a new serum biomarker to discriminate miscarriage from viable intrauterine pregnancy. Fertil Steril 2018; 109:137-141.e2.
10. Palmer S S, Barnhart K T. Biomarkers in reproductive medicine: the promise and can it be fulfilled? Fertil Steril 2013; 99:954-62.
11. Uhlen M, Fagerber L, Hallstrom B M, Lindskog C, Oksvold P, Mardinoglu A, et al. Proteomics. Tissue-based map of the human proteome. Science. 2015; 23: 347 (62220): 1260419.
12. Thomas P D, Ebert D, Muruganujan A, Mushayahama T, Laurent-Philippe A, Mi H. PANTHER: making genome-scale phylogenetics accessible to all. Protein Society 2022; 31:8-22.
13. Moldogazieva N T, Mokhosoev I M, Terentiev A A. Pregnancy-specific beta-1-glycoproteins: combined biomarker roles, structure/function relationships and implications for drug design. Curr Med Chem 2017; 24:245-67.
14. PSG9 pregnancy specific beta-1-glycoprotein 9 [Homo sapiens (human)]-Gene-NCBI. 2018. Available at: https://www.ncbi.nlm.nih.gov/gene/5678. Accessed Aug. 30, 2022.
15. Rong W, Yang L, Yin L, Gao Y, Xiao T, Cheng S. PSG9 promotes angiogenesis by stimulating VEGFA production and is associated with poor prognosis in hepatocellular carcinoma. 2017. Springer Link. Available at: https://link.springer.com/article/10.1007% 2Fs11427-016-0226-7. Accessed Jun. 3, 2022.
16. Yang L, Hu S, Tan J, Zhang X, Yuan W, Wang Q, et al. Pregnancy-specific glycoprotein 9 (PSG9), a driver for colorectal cancer, enhances angiogenesis via activation of SMAD4. Oncotarget 2016; 7:61562-61574.
17. Mueller M D, Raio L, Spoerri S, Ghezzi F, Dreher E, Bersinger N A. Novel placental and non-placental serum markers in ectopic versus normal intrauterine pregnancy. Fertil Steril 2004; 81:1106-11.
18. Horne C H, Towler C M, Pugh-Humphreys R G, Thomson A W, Bohn H. Pregnancy specific beta1-glycoprotein—a product of the syncytiotrophoblast. Experientia. 1976; 32:1197.
19. Earl U, Wells M, Bulmer J N. Immunohistochemical characterisation of trophoblast antigens and secretory products in ectopic tubal pregnancy. Int J Gynecol Pathol. 1986; 5:132-42.
20. Tornehave D, Chemnitz J, Westergaard J G, Teisner B, Poulsen H K, Bolton A E, et al. Placental proteins in peripheral blood and tissues of ectopic pregnancies. Gynecol Obstet Invest 1987; 23:97-102.
21. Witt B R, Wolf G C, Wainwright C J, Johnston P D, Thorneycroft I H. Relaxin, CA-125, progesterone, estradiol, Schwangerschaft protein, and human chorionic gonadotropin as predictors of outcome in threatened and nonthreatened pregnancies. Fertil Steril 1990; 53:1029-36.
22. Moore T, Dveksler G. Pregnancy-specific glycoproteins: complex gene families regulating maternal-fetal interactions.-PubMed-NCBI. 2014. Ncbi.nlm.nih.gov. Available at: https://www.ncbi.nlm.nih.gov/pubmed/25023693. Accessed Aug. 28, 2022.
23. Ha C, Wu J, Irmak S, Lisboa F, Dizon A, Warren J, et al. Human pregnancy specific beta-1-glycoprotein 1 (PSG1) has a potential role in placental vascular morphogenesis. National Center for Biotechnology Information. 2018. Available at: https://www.ncbi.nlm.nih.gov/pubmed/20335639. Accessed Jun. 1, 2022.
24. PSG1 pregnancy specific beta-1-glycoprotein 1 [Homo sapiens (human)]-Gene-NCBI. 2018. Available at: https://www.ncbi.nlm.nih.gov/gene/5669. Accessed Jun. 1, 2022.
25. Apweiler R, Bairoch A, Wu C H, Barker W C, Boeckmann B, Ferro S, et al. UniProt: the universal protein knowledgebase. Nucleic Acids Research 2004; 32: D115-9.
26. IGFBP1 insulin like growth factor binding protein 1 [Homo sapiens (human)]-Gene-NCBI. 2018. Available at: https://www.ncbi.nlm.nih.gov/gene/3484. Accessed Jun. 1, 2022.
27. Kyriacou C, Abbara A, Bobdiwala S, Fourie H, Al-Memar M, Phylactou M, et al. Circulating kisspeptin levels in ectopic pregnancy and pregnancy of unknown location. Ultrasound Obstet Gynecol 2020; 56:318.
28. Bilban M, Ghaffari-Tabrizi N, Hintermann E, Bauer S, Molzer S, Zoratti C, et al. Kisspeptin-10, a KiSS-1/metastin-derived decapeptide, is a physiological invasion inhibitor of primary human trophoblasts. 2004. Journal of Cell Science. Available at: http://jcs.biologists.org/content/117/8/1319.short. Accessed Jun. 1, 2022.
29. KISS1 KiSS-1 metastasis suppressor [Homo sapiens (human)]-Gene-NCBI. 2018. Available at: https://www.ncbi.nlm.nih.gov/gene/3814. Accessed Jun. 1, 2022.
30. Camolotto S, Racca A, Rena V, Nores R, Patrito L, Genti-Raimondi S, et al. Expression and transcriptional regulation of individual pregnancy-specific glycoprotein genes in differentiating trophoblast cells. Placenta 2010; 31:312-9.
31. Shanley D, Kiely P, Golla K, Allen S, Martin K, O'riordan R, et al. Pregnancy specific glycoproteins bind integrin aIIbb3 and inhibit the platelet-fibrinogen interaction. PLOS One 2013; 8: e57491.
32. PSG3 pregnancy specific beta-1-glycoprotein 3 [Homo sapiens (human)]-Gene-NCBI. 2018. Available at: https://www.ncbi.nlm.nih.gov/gene/5671. Accessed Jun. 1, 2022.
33. PSG3-Pregnancy-specific beta-1-glycoprotein 3 precursor—Homo sapiens (Human)-PSG3 gene & protein. 2018. Available at: https://www.uniprot.org/uniprot/Q16557. Accessed Jun. 1, 2022.
34. Robinson J F, Kapidzic M, Gormley M, Ona K, Dent T, Seifikar H, et al. Transcriptional dynamics of cultured human villous cytotrophoblasts. Endocrinology 2017; 158:1581-94.
35. Nicholson L, Madawala R, Lindsay L, Murphy C R. α-Parvin and β-parvin in the rat uterus during decidualisation and uterine receptivity. Histochem Cell Biol 2019; 151:395-406.
36. Liu J-L, Zhao M. Prioritization of susceptibility genes for ectopic pregnancy by gene network analysis. Int J Mol Sci 2016; 17:191.
37. Chen Q, Ni Y, Han M, Zhou W J, Zhu X B, Zhang A J. Integrin-linked kinase improves uterine receptivity formation by activating Wnt/β-catenin signaling and up-regulating MMP-3/9 expression. American journal of translational research 2020; 12:3011-22.
38. Kawano Y, Nakamura S, Nasu K, Fukuda J, Narahara H, Miyakawa I. Expression and 38. regulation of thrombospondin-1 by human endometrial stromal cells. Fertil Steril 2005; 83:1056-9.
39. Seppala M, Taylor R N, Koistinen H, Koistinen R, Milgrom E. Glycodelin: a major lipocalin protein of the reproductive axis with diverse actions in cell recognition and differentiation. Endocr Rev 2002; 23:401-30.
40. Foth D, Romer T. Glycodelin serum levels in women with ectopic pregnancy. Eur J Obstet Gynecol Reprod Biol 2003; 108:199-202.
41. Daponte A, Pournaras S, Zintzaras E, Kallitsaris A, Lialios G, Maniatis A N, et al. The value of a single combined measurement of VEGF, glycodelin, progesterone, PAPP-A, HPL and LIF for differentiating between ectopic and abnormal intrauterine pregnancy. Hum Reprod 2005; 20:3163-6.
42. Vigne J L, Hornung D, Mueller M D, Taylor R N. Purification and characterization of an immunomodulatory endometrial protein, glycodelin. J Biol Chem 2001; 276:17101-5.
43. Stabile I, Olajide F, Chard T, Grudzinkas J G. Circulating levels of placental protein 14 in ectopic pregnancy. British Journal of Obstetrics and Gynaecology 1994; 101:762-4.
44. Ruge S, Sorensen S, Vejtorp M, Vejerslev L O. The secretory endometrial protein, placental protein 14, in women with ectopic gestation. Fertil Steril 1992; 57:102-6.
45. Pedersen J F, Sorensen S, Ruge S. Serum level of secretory endometrial protein PP-14 in intact ectopic pregnancy. BJOG 1991; 98:414.
46. Lu Q, Insinna C, Ott C, Stauffer J, Pintado P, Rahajeng J, et al. Early steps in primary cilium assembly require EHD1/EHD3-dependent ciliary vesicle formation. 2015. Nature. Available at: https://www.nature.com/articles/ncb3109. Accessed Jun. 1, 2022.
47. Waxmonsky N, Conner S. Alphavbeta3-integrin-mediated adhesion is regulated through an AAKIL- and EHD3-dependent rapid-recycling pathway. 2013. Available at: http://jcs.biologists.org/content/126/16/3593. Accessed Jun. 1, 2022.
48. Dhiman P, Senthilkumar G P, Rajendiran S, Sivaraman K, Soundararaghavan S, Kulandhasamy M, Serum activin B concentration as predictive biomarker for ectopic pregnancy. Clinical biochemistry 2016; 49:609-12.
49. Refaat B, Bahathiq A O. The performances of serum activins and follistatin in the diagnosis of ectopic pregnancy: a prospective case-control study. Clin Chim Acta. 2020; 500:69-74.
50. Horne A W, van den Driesche S, King A E, Burgess S, Myers M, Ludlow H, et al. Endometrial inhibin/activin beta-B subunit expression is related to decidualization and is reduced in tubal ectopic pregnancy. J Clin Endocrinol Metab 2008; 93:2375-82.
51. Le Naour F, Rubinstein E, Jasmin C, Prenant M, Boucheix C. Severely reduced female fertility in CD9-deficient mice. Science 2000; 287:319-21.
52. Higginbottom A, Takahashi Y, Bolling L, Coonrod S, White J, Partridge L. et al. Structural requirements for the inhibitory action of the CD9 large extracellular domain in sperm/oocyte binding and fusion. Elsevier. 2003. Available at: https://www.sciencedirect.com/science/article/pii/S0006291X03020321?via % 3Dihub. Accessed Jun. 1, 2022.
53. Nakazawa Y, Sato S, Naito M, Kato Y, Mishima K, Arai H, et al. Tetraspanin family member CD9 inhibits Aggrus/podoplanin-induced platelet aggregation and suppresses pulmonary metastasis. 2008. American Society of Hematology. Available at: http://www.bloodjournal.org/content/112/5/1730.long?sso-checked=true. Accessed Jun. 1, 2022.
54. CD9 molecule [Homo sapiens (human)]-Gene-NCBI. 2018. Available at: https://www.ncbi.nlm.nih.gov/gene/928. Accessed Jun. 1, 2022.
55. Dou Q, Williams R S, Chegini N. Expression of integrin messenger ribonucleic acid in human endometrium: a quantitative reverse transcription polymerase chain reaction study. Fertil Steril 1999; 71:347-53.
56. Apparao K B, Murray M J, Fritz M A, Meyer W R, Chambers A F, Truong P R, et al. Osteopontin and its receptor alphavbeta (3) integrin are coexpressed in the human endometrium during the menstrual cycle but regulated differentially. J Clin Endocrinol Metab 2001; 86:4991-5000.
57. Lessey B A. Endometrial integrins and the establishment of uterine receptivity, Hum Reprod 1998; 13:247-58.
58. Sulz L, Valenzuela J P, Salvatierra A M, Ortiz M E, Croxatto H B. The expression of alpha (v) and beta3 integrin subunits in the normal human Fallopian tube epithelium suggests the occurrence of a tubal implantation window. Hum Reprod 1998; 3:2916-20.
59. Inan S, Giray G, Vatansever H S, Ozbilgin K, Kuscu N K, Sayhan S. Immunolocalization of integrins and fibronectin in tubal pregnancy. Acta Histochem 2004; 106:235-43.
60. Beer L A, Senapati S, Sammel M D, Barnhart K T, Schreiber C A, Speicher D W, et al. Proteome-defined changes in cellular pathways for decidua and trophoblast tissues associated with location and viability of early-stage pregnancy. Reprod Biol Endocrinol 2022; 20:36.
61. Yen C F, Wang H S, Lee C L, Liao, S K. Roles of integrin-linked kinase in cell signaling and its perspectives as a therapeutic target. Gynecol Minim Invasive Ther 2014; 3:67-72.
62. Elustondo P A, Hannigan G E, Caniggia I, MacPhee D J. Integrin-linked kinase (ILK) is highly expressed in first trimester human chorionic villi and regulates migration of a human cytotrophoblast-derived cell line. Biol Reprod 2006; 74:959-68.
63. Soleilhavoup C, Riou C, Tsikis G, et al. Proteomes of the female genital tract during the oestrous cycle. Mol Cell Proteomics 2016; 15:93-108.
64 Zhao Y, Kan F W K. Human OVGP1 enhances tyrosine phosphorylation of proteins in the fibrous sheath involving AKAP3 and increases sperm-zona binding. J Assist Reprod Genet 2019; 36:1363-77.
65. Zhao Y, Vanderkooi S, Kan F W K. The role of oviduct-specific glycoprotein (OVGP1) in modulating biological functions of gametes and embryos. Histochem Cell Biol 2022; 157:371-88.
66. Hao W, Xiaowei Z, Chunfeng L, Shengfu C, Xinyang L, Shangrong F. TAGLN2-Regulated Trophoblast Migration, Invasion and Fusion are Impaired in Preeclampsia. Front Cell Dev Biol 2022; 10:810633.
67. Liang X, Jin Y, Wang H, Meng X, Tan Z, Huang T, et al. Transgelin 2 is required for embryo implantation by promoting actin polymerization. FASEB J 2019; 33:5667-75.
68. Barnhart K, van Mello N M, Bourne T, Kirk E, Van Calster B, Bottomley C, et al. Pregnancy of unknown location: a consensus statement of nomenclature, definitions, and outcome. Fertil Steril. 2011; 95:857-66.
69. Chetty M, Sawyer E, Dew T, Chapman A J, Elson J. The use of novel biochemical markers in predicting spontaneously resolving ‘pregnancies of unknown location. Hum Reprod 2011; 26:1318-23.

### Example 5: Additional Validation of Biomarkers

In this experiment, the use of multiplexed biomarkers were analyzed to determine their effectiveness for improving the diagnosis of normal or abnormal early pregnancies. 24 markers were assessed with multiple machine learning-based methodologies to evaluate combinations of top candidates to develop a multiplexed prediction model for identification of 1) viability and 2) location of an early pregnancy.

In this example, we have demonstrated a pool of biomarkers from divergent biological pathways that can be used to classify individuals with potential early pregnancy loss. The biomarkers CG-Alpha, PAPPA and PSG3 can be used to predict viability and sFLT, TPFI2 and PSG3 can be used to predict pregnancy location.

### Materials and Methods

### Participants

Participants in the study cohort were selected from a prospective cohort [10,11,13]. Inclusion consisted of 1) complaints of abdominal pain, vaginal bleeding, or both; 2) serum hCG of 50-100,000 mIU/mL; and 3) 4-10 weeks gestation by ultrasound or last menstrual period. Serum biobank samples were collected from individuals presenting for care due to risk of early pregnancy failure at clinical care settings at the Hospital of the University of Pennsylvania, Northwestern University, or Eastern Virginia Medical School. Ultrasound or results for surgical intervention was used to definitively determine the phenotype of the pregnancy at the time of serum collection or within 48 hours of serum collection using international consensus via chart review (14).

The design of this study was a nested case-control design consisting of 218 individuals (75 IUPs, 68 EPs, and 75 EPLs). Serum samples from subjects within the prospective cohort were selected to be representative of each of the three outcomes (not to be similar across outcomes). Participants were excluded if 1) they had received treatment during prior to enrollment; 2) had evidence of gestational trophoblastic disease; 3) were diagnosed with a non-tubal ectopic pregnancy; or 4) evidence of multiple gestation. All individuals conceived without medical assistance and were not taking supplemental hormones. Each subject had three aliquots of 510 μl serum each available for analysis. All samples were de-identified and stored at −80 degrees Celsius.

### Analysis of Biomarkers

Immunoassays were performed at University of Virginia Ligand Assay and Analysis Core. All 24 biomarkers were assessed on the same specimen for each subject. The rational for each marker, the manufacturer of the assay and intra assay coefficient of variation are listed in Table 10.

### Statistical Analysis

Differences in baseline characteristics of subjects between groups were evaluated using the two sample t-test for continuous measures, and Pearson Chi-square or Fisher-exact tests for categorical variables. For each biomarker, the concentration was compared to assess ability to predict viability (IUP vs EPL+EP) and location (EP vs EPL+IUP). The biomarkers with the strongest association in the univariate analyses, were evaluated in multivariate analyses to assess prediction of each outcome: 10 for viability and 11 for location.

### Classification Regression Tree Analysis (CART)

Based on our previous data we started with classification and regression tree analysis with Minitab (Minitab Version 21, Minitab, LLC. (2021)). Trees were created in two ways. One set of trees balanced sensitivity and specificity using an equal cost for misclassification of a false positive or false negative. Additionally, separate trees were created with a cost of misdiagnosis set at 5 or higher to maximize sensitivity (and a set that maximized specificity). All trees were created using 5-fold cross validation (An Easy Guide to K-Fold Cross-Validation—Statology).

The optimized trees in terms of sensitivity and specificity were used to define a two-step prediction to maximize accuracy (10,13,15). If the same prediction classification (e.g., viable [IUP] or nonviable [EPL+EP]) was achieved with both the tree that maximized specificity and the tree that maximized sensitivity, the prediction was classified accordingly (e.g., viable or nonviable). If the two trees predicted different outcomes, the prediction was considered indeterminate, and a classification was not made (not conclusively classified). This process was conducted independently for the prediction of viability and also separately (with different trees and different markers) to predict the location of a gestation (EP vs IUP+EPL). The predicted classification by each model was compared to actual diagnosis and sensitivity, specificity, positive predictive value (PPV), negative predictive value (NPV), conclusive classification, and accuracy statistics were calculated.

### Random Forest Analysis (RF)

We also assessed the predictive ability of the biomarkers with models derived using SVM-Polynominal, SVM-Linear, SVM-RBF, Logistic-regression, logistic regression with lasso, logistic regression with elasticnet, random forest and deep neural network using Python. We found the RF model consistently outperformed the other methods.

The random forest analysis used the same two classification tasks: viability and location. The dataset was partitioned into five equally sized folds: three for training, one for tuning the decision threshold, and one for testing. In the training set, we employed nested five-fold cross validation to optimize hyperparameters. The model was trained on four of the five folds and computed AUC-ROC on the remaining validation fold. This process was done iteratively five times, averaging the five AUC-ROC scores (FIG. 4).

Random forest models were created by adjusting the decision thresholds to maximize both sensitivity and specificity, subject to an accuracy constraint >0.8. As described above, the optimized trees for sensitivity and specificity were used a two-step prediction to maximize accuracy (10,13,15). If the same prediction classification was achieved with both the tree that maximized specificity and the tree that maximized sensitivity, the prediction was classified accordingly. If the two trees predicted different outcomes a classification was not made. The training-validation tuning processes was repeated 100 times and reported the mean and standard deviation of the testing performance. Analyses to investigate the predictive performance of the random forest model when limited to a subset of biologically plausible and agonistically selected markers were performed.

### Combined Use of Models for Viability and Location

The final CART and RF models to predict viability and location were assessed serially. If the viability test reported normal (IUP), then the outcome was predicted to be normal. If viability test reported abnormal (EP or EPL), then outcome was predicted to be abnormal (not viable). If viability test reported inconclusive and location test reported inconclusive, then outcome was predicted as inconclusive. If the viability test reported inconclusive and the location test reported EP, then the outcome was predicted as abnormal (not viable). If the viability test reported inconclusive and the location test reported non-EP (IUP or EPL), then the outcome was predicted as inconclusive.

### Results

The comparisons of demographics of the subjects across outcomes are presented in Table 17. The concentration of each of the 24 biomarkers and its ability to predict location and viability are presented in Table 12. Eleven markers were noted to be predictive, when used singly, with an AUC of prediction >0.7 and a P value <0.001 for prediction of viability or location (or both) (PSG1, sFLT, GDF15 PSG9, hCG, CG-Alpha, ADAM12, ANGPT2, TFPI2, PSG3, and PRG) (Table 13 and 18) and were used in combination to predict outcome.

TABLE 17
Baseline Demographics

| | Viability | | | | | Location | | | | |
|---|---|---|---|---|---|---|---|---|---|---|
| | IUP | | EPL & EP | | | EP | | IUP & EPL | | |
| | (n = 75) | | (n = 143) | | p value | (n = 68) | | (n = 150) | | p value |
| Race, n (%) | | | | | 0.35 | | | | | 0.41 |
| Asian | 4 | (5.3%) | 6 | (4.2%) | | 3 | (4.4%) | 7 | (4.7%) | |
| Black | 56 | (74.7%) | 93 | (65.0%) | | 42 | (61.8%) | 107 | (71.3%) | |
| White | 10 | (13.3%) | 33 | (23.1%) | | 18 | (26.5%) | 25 | (16.7%) | |
| Unknown | 5 | (6.7%) | 11 | (7.7%) | | 5 | (7.4%) | 11 | (7.3%) | |
| Ethnicity n(%) | | | | | 0.45 | | | | | 0.61 |
| Hispanic | 3 | (4.0%) | 11 | (7.7%) | | 6 | (8.8%) | 8 | (5.3%) | |
| Non-Hispanic | 69 | (92.0%) | 129 | (90.2%) | | 60 | (88.2%) | 138 | (92.0%) | |
| Unknown | 3 | (4.0%) | 3 | (2.1%) | | 2 | (2.9%) | 4 | (2.7%) | |
| hCG (mIU/mL), | 40413.0 | | 13783.2 | | <0.001 | 9000.3 | | 29451.1 | | <0.001 |
| mean (SD) | (18524.1) | | (17244.0) | | | (14707.9) | | (21511.5) | | |
| GA based on | 45.4 | (4.4) | 50.0 | (10.4) | 0.002 | 41.4 | (4.2) | 48.6 | (8.7) | 0.007 |

Ultrasound
(days), mean
(SD)

* IUP, intrauterine pregnancy; EPL, pregnancy loss; EP, ectopic pregnancy; hCG, human chorionic gonadotropin; SD, standard deviation; GA, gestational age
** Data are presented as mean (SD) for continuous measures using the Two-sample t test and n (%) for categorical measures using Pearson's chi-squared or Fisher's exact test.

TABLE 18
Biomarker Performance: Viability (IUP vs. EP and EPL)

| | | | | Std | | | | |
|---|---|---|---|---|---|---|---|---|
| Biomarker | Outcome | N | Mean | Dev | Median | Min | Max | AUC |
| Activin | IUP | 75 | 0.421 | 0.262 | 0.346 | 0.133 | 1.676 | 0.394 |
| A | Non | 143 | 0.526 | 0.34 | 0.43 | 0.147 | 2.281 | |
| | Via | | | | | | | |
| ADAM12 | IUP | 75 | 0.845 | 0.647 | 0.694 | 0.156 | 4.326 | 0.474 |
| | Non | 143 | 1.006 | 1.037 | 0.765 | 0.156 | 10.015 | |
| | Via | | | | | | | |
| ANGPT2 | IUP | 75 | 5542.94 | 4821.8 | 4046.75 | 1985.53 | 30000 | 0.491 |
| | Non | 143 | 5637.52 | 4488.65 | 4345.44 | 470 | 30000 | |
| | Via | | | | | | | |
| PRG | IUP | 75 | 13.724 | 5.622 | 13 | 0.1 | 33.3 | 0.817 |
| | Non | 142 | 7.729 | 6.891 | 6.045 | 0.74 | 49.9 | |
| | Via | | | | | | | |
| sFLT | IUP | 74 | 2520.43 | 1724.39 | 2297.83 | 370.41 | 11183.34 | 0.71 |
| | Non | 142 | 1744.4 | 2074.23 | 1041.64 | 288.41 | 16869.94 | |
| | Via | | | | | | | |
| PSG1 | IUP | 75 | 82.043 | 67.024 | 68.203 | 3.2 | 325.592 | 0.657 |
| | Non | 143 | 59.965 | 75.275 | 25.481 | 3.2 | 381.58 | |
| | Via | | | | | | | |
| PSG3 | IUP | 75 | 64.436 | 84.794 | 37.207 | 9.207 | 457.994 | 0.902 |
| | Non | 143 | 17.201 | 36.88 | 9.299 | 0.625 | 409.132 | |
| | Via | | | | | | | |
| PSG9 | IUP | 75 | 23141.04 | 12934.82 | 21966.5 | 826.05 | 87952.3 | 0.868 |
| | Non | 143 | 7687.57 | 11542.61 | 3206.15 | 85.263 | 73974.2 | |
| | Via | | | | | | | |
| NOTUM | IUP | 75 | 0.998 | 0.586 | 0.78 | 0.78 | 3.6 | 0.543 |
| | Non | 143 | 0.964 | 0.622 | 0.78 | 0.78 | 5.098 | |
| | Via | | | | | | | |
| PAPPA | IUP | 75 | 0.734 | 2.352 | 0.39 | 0.39 | 20.644 | 0.385 |
| | Non | 143 | 0.733 | 1.046 | 0.39 | 0.39 | 9.977 | |
| | Via | | | | | | | |
| PLGF | IUP | 74 | 15.787 | 16.434 | 13.919 | 7.777 | 149.12 | 0.498 |
| | Non | 143 | 15.07 | 8.241 | 12.951 | 7.777 | 68.006 | |
| | Via | | | | | | | |
| EHD3 | IUP | 75 | 14.014 | 17.039 | 7.018 | 3.1 | 100 | 0.392 |
| | Non | 143 | 18.367 | 18.163 | 11.954 | 3.1 | 100 | |
| | Via | | | | | | | |
| Fibronectin | IUP | 75 | 234968.1 | 62286.33 | 221531.6 | 120458.2 | 414139.3 | 0.555 |
| | Non | 143 | 220538.1 | 67567.21 | 218401.2 | 16658.24 | 522893.1 | |
| | Via | | | | | | | |
| hCG | IUP | 75 | 39640.76 | 19792.9 | 40219 | 930 | 112645 | 0.85 |
| | Non | 143 | 13822.15 | 18083.4 | 6829 | 269 | 89776 | |
| | Via | | | | | | | |
| Kisspeptin | IUP | 75 | 6.575 | 7.779 | 2.746 | 0.496 | 25 | 0.544 |
| | Non | 143 | 5.403 | 6.719 | 2.623 | 0.02 | 25 | |
| | Via | | | | | | | |
| IGFBP1 | IUP | 75 | 8833.6 | 7393.6 | 5995.5 | 1425.1 | 42204.9 | 0.33 |
| | Non | 143 | 20392 | 21479.5 | 11840.1 | 630 | 80000 | |
| | Via | | | | | | | |
| GDF15 | IUP | 75 | 5039.9 | 2809.01 | 4808.5 | 724 | 15000 | 0.83 |
| | Non | 143 | 2208.2 | 2188.5 | 1359.7 | 117 | 14941.6 | |
| | Via | | | | | | | |
| HAGH | IUP | 75 | 15.265 | 4.451 | 14.447 | 6.38 | 32 | 0.648 |
| | Non | 143 | 13.202 | 4.567 | 12.176 | 1.2 | 30.2 | |
| | Via | | | | | | | |
| CD9 | IUP | 70 | 1.317 | 2.789 | 0.316 | 0.31 | 20 | 0.485 |
| | Non | 142 | 1.248 | 2.316 | 0.331 | 0.31 | 14.87 | |
| | Via | | | | | | | |
| TFPI2 | IUP | 74 | 234.02 | 317.41 | 137.21 | 41.97 | 1778.4 | 0.448 |
| | Non | 142 | 282.67 | 322.52 | 172.05 | 39 | 1855.39 | |
| | Via | | | | | | | |
| SIGLEC-6 | IUP | 75 | 2247.24 | 3180.29 | 1126.09 | 243.41 | 21923.91 | 0.629 |
| | Non | 142 | 2783.1 | 5712.64 | 664.46 | 237 | 30000 | |
| | Via | | | | | | | |
| OPN | IUP | 74 | 7.42 | 4.97 | 6.01 | 2.18 | 25.49 | 0.357 |
| | Non | 142 | 10.57 | 8.47 | 8.86 | 1.69 | 60 | |
| | Via | | | | | | | |
| ELAFIN | IUP | 75 | 10727.82 | 10819.46 | 6563.47 | 368.7 | 40000 | 0.554 |
| | Non | 143 | 9610.53 | 10281.88 | 6053.64 | 747.8 | 40000 | |
| | Via | | | | | | | |
| CG- | IUP | 75 | 13350.76 | 7811.66 | 12914.48 | 500 | 48954.23 | 0.865 |
| ALpha | Non | 143 | 4227.34 | 6436.17 | 1585.44 | 500 | 41756.2 | |
| | Via | | | | | | | |

| | | | | T- | | | |
|---|---|---|---|---|---|---|---|
| | | | AUC | test | Ranksum | % | |
| | | 95% | (Same | p- | p- | Not | |
| Biomarker | Outcome | CI | Direction) | value | value | Detected | Direction |
| Activin | IUP | 0.317-0.471 | 0.606 | 0.02 | 0.01 | 0.00% | Down |
| A | Non | | | | | 0.00% | in |
| | Via | | | | | | IUP |
| ADAM12 | IUP | 0.398-0.551 | 0.526 | 0.222 | 0.531 | 1.33% | Down |
| | Non | | | | | 4.20% | in |
| | Via | | | | | | IUP |
| ANGPT2 | IUP | 0.413-0.570 | 0.509 | 0.886 | 0.834 | 1.33% | Down |
| | Non | | | | | 1.40% | in |
| | Via | | | | | | IUP |
| PRG | IUP | 0.760-0.874 | 0.817 | <0.0001 | <0.0001 | 1.33% | Up in |
| | Non | | | | | 0.00% | IUP |
| | Via | | | | | | |
| sFLT | IUP | 0.641-0.779 | 0.71 | 0.006 | <0.0001 | 0.00% | Up in |
| | Non | | | | | 0.00% | IUP |
| | Via | | | | | | |
| PSG1 | IUP | 0.585-0.728 | 0.657 | 0.034 | 0.0001 | 4.00% | Up in |
| | Non | | | | | 25.17% | IUP |
| | Via | | | | | | |
| PSG3 | IUP | 0.863-0.941 | 0.902 | <0.0001 | <0.0001 | 0.00% | Up in |
| | Non | | | | | 0.00% | IUP |
| | Via | | | | | | |
| PSG9 | IUP | 0.818-0.918 | 0.868 | <0.0001 | <0.0001 | 0.00% | Up in |
| | Non | | | | | 0.00% | IUP |
| | Via | | | | | | |
| NOTUM | IUP | 0.489-0.597 | 0.543 | 0.694 | 0.089 | 78.67% | Up in |
| | Non | | | | | 89.51% | IUP |
| | Via | | | | | | |
| PAPPA | IUP | 0.325-0.445 | 0.615 | 0.996 | 0.0008 | 80.00% | Down |
| | Non | | | | | 60.84% | in |
| | Via | | | | | | IUP |
| PLGF | IUP | 0.418-0.578 | 0.502 | 0.669 | 0.965 | 6.76% | Down |
| | Non | | | | | 4.90% | in |
| | Via | | | | | | IUP |
| EHD3 | IUP | 0.313-0.472 | 0.608 | 0.088 | 0.009 | 13.33% | Down |
| | Non | | | | | 7.69% | in |
| | Via | | | | | | IUP |
| Fibronectin | IUP | 0.476-0.633 | 0.555 | 0.126 | 0.185 | 0.00% | Up in |
| | Non | | | | | 0.00% | IUP |
| | Via | | | | | | |
| hCG | IUP | 0.795-0.904 | 0.85 | <0.0001 | <0.0001 | 0.00% | Up in |
| | Non | | | | | 0.00% | IUP |
| | Via | | | | | | |
| Kisspeptin | IUP | 0.463-0.624 | 0.544 | 0.248 | 0.29 | 10.67% | Up in |
| | Non | | | | | 8.39% | IUP |
| | Via | | | | | | |
| IGFBP1 | IUP | 0.259-0.402 | 0.67 | <0.0001 | <0.0001 | 0.00% | Down |
| | Non | | | | | 7.69% | in |
| | Via | | | | | | IUP |
| GDF15 | IUP | 0.776-0.884 | 0.83 | <0.0001 | <0.0001 | 1.33% | Up in |
| | Non | | | | | 0.70% | IUP |
| | Via | | | | | | |
| HAGH | IUP | 0.575-0.722 | 0.648 | 0.002 | 0.0003 | 1.33% | Up in |
| | Non | | | | | 0.70% | IUP |
| | Via | | | | | | |
| CD9 | IUP | | 0.515 | 0.848 | 0.707 | 51.43% | Down |
| | Non | 0.406-0.564 | | | | 47.18% | in |
| | Via | | | | | | IUP |
| TFPI2 | IUP | 0.370-0.527 | 0.552 | 0.291 | 0.212 | 0.00% | Down |
| | Non | | | | | 1.41% | in IUP |
| | Via | | | | | | |
| SIGLEC-6 | IUP | 0.557-0.702 | 0.629 | 0.453 | 0.002 | 0.00% | Up in |
| | Non | | | | | 20.42% | IUP |
| | Via | | | | | | |
| OPN | IUP | 0.280-0.434 | 0.643 | 0.004 | 0.0006 | 0.00% | Down |
| | Non | | | | | 1.41% | in IUP |
| | Via | | | | | | |
| ELAFIN | IUP | 0.476-0.633 | 0.554 | 0.455 | 0.188 | 6.67% | Up in |
| | Non | | | | | 8.39% | IUP |
| | Via | | | | | | |
| CG- | IUP | 0.814-0.917 | 0.865 | <0.0001 | <0.0001 | 1.33% | Up in |
| ALpha | Non | | | | | 10.49% | IUP |
| | Via | | | | | | |

### Classification Regression Tree

Feature selection using CART demonstrated PSG3, PSG9, CG_Alpha, hCG, GDF15, and PRG were most predictive of viability and PSG1, sFLT, GDF15, PSG9, hCG, and CG_ALPHA were most predictive of location.

Viability: The best models to predict viability used three makers (PSG3, CG-Alpha and PAPPA). The maximum sensitivity and specificity achieved by a model were 93.3% and 98.6 respectively. The model with the highest accuracy was 97.4% (with 70.2% receiving classification). (Table 18)

Location: The best models to predict location used three markers (sFLT, PSG3 and TFP12). The maximum sensitivity and specificity achieved by a model were 98.5% and 95.3 respectively. The model with the highest accuracy was 94.4% (with 65.6% receiving classification). (Table 19)

When the models for viability and location were used serially, the number receiving classification increased to 72.7% with accuracy of 95.9%.

TABLE 18
Performance of CART Models to Predict Viability (IUP vs. EPL + EP) with 95% CI

| | | | | | | | Accuracy |
|---|---|---|---|---|---|---|---|
| | | | | | | % | Amongst |
| Model | Markers | Sensitivity | Specificity | PPV | NPV | Classified | Classified |
| Balance of | PSG3 | 93.3% | 76.2% | 67.3% | 95.6% | 100% | 82.1% |
| sensitivity | | (87.7, 99.0) | (69.3, 83.2) | (58.3, 76.3) | (91.9, 99.4) | (100.0, 100.0) | (77.0, 87.2) |
| and | | | | | | | |
| specificity | | | | | | | |
| ¹Maximum | PSG3 | 93.3% | 76.2% | 67.3% | 95.6% | 100% | 82.1% |
| Sensitivity | | (87.7, 99.0) | (69.3, 83.2) | (58.3, 76.3) | (91.9, 99.4) | (100.0, 100.0) | (77.0, 87.2) |
| Maximum | PSG3 | 57.3% | 97.9% | 93.5% | 81.4% | 100% | 83.9% |
| Specificity | PAPPA | (46.1, 68.5) | (95.6, 100.0) | (86.3, 100.0) | (75.6, 87.2) | (100.0, 100.0) | (79.1, 88.8) |
| Maximum | PSG3 | 54.7% | 75.5% | 97.6% | 97.3% | 70.2% | 97.4% |
| Accuracy | CG_{—}Alpha | (43.4, 65.9) | (68.5, 82.6) | (93.0, 100.0) | (94.3, | (64.1, 76.3) | (94.9, 99.9) |
| | PAPPA | | | | 100.0) | | |

The bolded values highlight the test characteristics that were optimized in each model.
The bolded model is the preferred model.
Maximum Accuracy model uses both the Maximum Sensitivity and Maximum Specificity models.
¹The Balanced model, while balancing sensitivity and specificity, happens to maximize sensitivity, resulting in identical values to those of the Maximum Sensitivity model.

TABLE 19
Performance of CART Models to Predict Location (EP vs. EPL + IUP) with 95% CI

| | | | | | | | Accuracy |
|---|---|---|---|---|---|---|---|
| | | | | | | | Amongst |
| Model | Marker(s) | Sensitivity | Specificity | PPV | NPV | % Classified | Classified |
| Balance | PSG1 | 82.4% | *84.0% | 70 0% | 91.3% | 100% | 83.5% |
| of | | (73.3, 91.5) | (78.1, 89.9) | (60 0, 80 0) | (86 6, 96.0) | 100.0, 100.0) | (78,6, 88.4) |
| sensitivity | | | | | | | |
| and | | | | | | | |
| specificity | | | | | | | |
| Maximum | PSG3 | 98.5% | 61.3% | 53.6% | 98.9% | 100% | 72.9% |
| Sensitivity | TFPI2 | (95.7, 100.0) | (53.5, 69.1) | (44.9, 62.3) | (94.2, 99.9) | (100.0, 100 | (67.0, 78.8) |
| | | | | | | .0) | |
| Maximum | SFLT | 63,2% | 95.3% | 86 0% | 85.1% | 100% | 85.3% |
| Specificity | | (51 8, 74.7) | (92.0, 98.7) | (76,4, 95.6) | (79 7, 90 5) | (100.0, 100.0) | 80,6, 90,0) |
| Maximum | PSG3 | 63.2% | 61.3% | 86.0% | 98.9% | 65.6% | 94.4% |
| Accuracy | SFLT | (51.8, 74.7) | (53.5, 69.1) | (76.4, 95.6) | (96.8, 100.0) | (59.3, 71.9) | (90.6, 98.2) |
| | TFP12 | | | | | | |

The bolded values highlight the test characteristics that were optimized in each model
The bolded model is the preferred model.
Maximum Accuracy model uses both the Maximum Sensitivity and Maximum Specificity models

### Random Forest

Viability: RF used 10 markers to predict viability. The maximum sensitivity and specificity achieved by the models were 99% and 95% respectively. The model with the highest accuracy was 97% (with 65% receiving classification). (Table 20). When the number of markers was reduced accuracy decreased. When the model was restricted to 6 markers the accuracy was 95% (with 69% receiving classification), and when only two markers were the accuracy was 91% (73% classified).

TABLE 20
Performance of Random Forest (RF) Model to Predict Viability (IUP vs EPL + EP)

| | | | | | | | Accuracy |
|---|---|---|---|---|---|---|---|
| | | | | | | % | Amongst |
| Model | Markers | Sensitivity | Specificity | PPV | NPV | Classified | Classified |
| Balance of | All 10 | 91.8% | 73.1% | 88.0% | 84.0% | 100% | 85.6% |
| sensitivity | | (6.7%) | (18.5%) | (6.7%) | (11.7%) | | (5.4%) |
| and | | | | | | | |
| specificity | | | | | | | |
| Maximum | All 10 | 99.1% | 38.8% | 76.8% | 95.4% | 100% | 79.0% |
| Sensitivity | | (1.9%) | (19.1%) | (5.7%) | (7.1%) | | (6.0%) |
| Maximum | All 10 | 74.7% | 94.9% | 97.2% | 66.6% | 100% | 81.4% |
| Specificity | | (11.2%) | (10.5%) | (4.5%) | (10.5%) | | (6.6%) |
| § Maximum | All 10 | 98.9% | 88.4% | 97.2% | 95.4% | 65.0% | 96.9% |
| Accuracy | | (2.3%) | (16.4%) | (4.5%) | (7.1%) | (11.8%) | (4.4%) |
| Maximum | PSG3 | 97.2% | 84.1% | 95.2% | 93.5% | 69.6% | 94.2% |
| Accuracy | PSG9 | (5.9%) | (18.1%) | (5.8%) | (12.6%) | (13.9%) | (6.1%) |
| with 6 | hCG | | | | | | |
| Markers | CG_Alpha | | | | | | |
| | GDF15 | | | | | | |
| | ADAM12 | | | | | | |
| Maximum | PSG3 | 90.5% | 92.0% | 97.1% | 78.9% | 72.9% | 91.0% |
| Accuracy | hCG | (7.5%) | (12.8%) | (4.7%) | (13.3%) | (14.4%) | (6.1%) |
| with 2 | | | | | | | |
| Markers | | | | | | | |

The performance was reported in terms of the mean and standard deviation of the 100 testing results.
The bolded values highlight the test characteristics that were optimized in each model.
The bolded model is the preferred model.
“All 10” refers to the markers: PSG3, PSG9, PSG1, hCG, CG_Alapha, GDF15, sFLT, PRG, PAPPA, and ADAM 12.
Maximum Accuracy refers to a cross of the Maximum Sensitivity and Maximum Specificity models.

Location: RF used 11 markers to predict location. The maximum sensitivity and specificity achieved by a model were 97% and 81% respectively. The model with the highest accuracy was 89% (with 75% receiving classification) (Table 21). When the models were restricted to 6 markers the accuracy was 89% (76% receiving classification) and when only two markers were used the accuracy was 89% (78% classified).

When the models for viability and location were used serially the number receiving classification increased to 81.0% with accuracy of 94.1%.

TABLE 21
Performance of Random Forest (RF) Model to Predict Location (EP vs. EPL + IUP

| | | | | | | | Accuracy |
|---|---|---|---|---|---|---|---|
| | | | | | | % | Amongst |
| Model | Markers | Sensitivity | Specificity | PPV | NPV | Classified | Classified |
| Balance of | All 11 | 93.0% | 62.2% | 85.1% | 82.3% | 100% | 83.5% |
| sensitivity | | (6.0%) | (17.1%) | (5.6%) | (12.0%) | | (5.0%) |
| and | | | | | | | |
| specificity | | | | | | | |
| Maximum | All 11 | 96.6% | 41.1% | 79.0% | 86.7% | 100% | 79.4% |
| Sensitivity | | (4.1%) | (20.0%) | (5.8%) | (13.8%) | | (5.5%) |
| Maximum | All 11 | 78.4% | 81.4% | 90.8% | 65.4% | 100% | 79.3% |
| Specificity | | (11.7%) | (11.8%) | (5.1%) | (12.6%) | | (7.4%) |
| Maximum | All 11 | 95.8% | 67.5% | 90.8% | 86.7% | 75.0% | 89.4% |
| Accuracy | | (4.9%) | (20.1%) | (5.1%) | (13.8%) | (12.8%) | (4.9%) |
| Maximum | PSG3 | 96.0% | 62.2% | 89.6% | 85.8% | 75.7% | 88.6% |
| Accuracy | PSG9 | (4.3%) | (22.5%) | (5.0%) | (16.3%) | (13.1%) | (5.1%) |
| with 6 | PSG1 | | | | | | |
| Markers | hCG | | | | | | |
| | GDF15 | | | | | | |
| | sFLT | | | | | | |
| Maximum | PSG3 | 96.1% | 65.8% | 89.7% | 88.8% | 77.5% | 88.9% |
| Accuracy | hCG | h(C4G.9%) | (18.8%) | (5.2%) | (12.6%) | (12.4%) | (5.5%) |
| with 2 | | | | | | | |
| Markers | | | | | | | |

The performance was reported in terms of the mean and standard deviation of the 100 testing results.
The bolded values highlight the test characteristics that were optimized in each model.
The bolded model is the preferred model.
“All 11” refers to the markers: PSG3, PSG9, PSG1, hCG, CG_Alpha, GDF15, sFLT, PRG, PAPPA, ADAM 12, and TFP12.
Maximum Accuracy refers to a cross of the Maximum Sensitivity and Maximum Specificity models.

### Results

We have demonstrated that a small group of biomarkers from different biological pathways can be combined to accurately predict the viability of an early pregnancy. An overlapping group of biomarkers can predict the location of a pregnancy. The biomarkers have been narrowed from a large number of potential markers that were identified by agnostic discovery and biological plausibility. The proposed biological pathways include trophoblast cell function, angiogenesis, cell adhesion and extracellular matrix constituents. Prediction using a variety of machine learning techniques varies modestly depending on the number of markers used in each model. Models derived with CART were the most parsimonious and logical. Accurate prediction can be obtained using CART with 3 markers for either viability (PSG3, PAPPA and CG-Alpha) or location (PSG3, sFLT, TPRI2) of an early gestion.

A companion diagnostic using multiplexed biomarkers needs to balance accuracy with simplicity and parsimony (17). There is a dilemma regarding whether a desired companion diagnostic for early pregnancy loss (4.9%) sizes sensitivity, maximizes specificity, or balance the two. These data demonstrate that models can be adjusted to maximize the desired test characteristics. Such decisions will have advantages and limitations. Maximizing sensitivity will identify more individuals with an ectopic pregnancy or nonviable pregnancy but will increase the number intrauterine or viable pregnancies falsely classified as nonviable or ectopic. Maximizing specificity will identify more individuals with an intrauterine pregnancy or a viable pregnancy but will increase the number of individuals with an ectopic pregnancy or nonviable pregnancy classified as normal. Because there are consequences of both false positive and false negative prediction, we advocate the models presented that maximize accuracy. Those who do not receive a conclusive classification can be followed with standard of care, minimizing inaccurate diagnosis.

The first step in developing a diagnostic model is to identify and validate the most promising candidates (8). The biomarkers associated with divergent pathways of the biology of early pregnancy synergize allow better prediction. We have previously demonstrated that the use of multiple markers enhances prediction over the use of any single marker (10,13). Upon finalizing a relatively small group of high priority candidates we explored multiple methods of machine learning to identify the most promising models. While the pathology of miscarriage and an ectopic pregnancy may overlap, some markers were more predictive of one outcome or the other. Ten of eleven makers contributed to both the prediction of viability and location.

The model using CART uses the fewest markers and is the most intuitive. Viability can be predicted with 97.4% accuracy using three markers. Location can be predicted with 94.4% accuracy using three makers. Exploration of other computer learning models demonstrated that random forest consistently produced better results than the use of logistic regression or deep neural networks. Models using random forest were modestly more accurate than models using CART (97% accuracy for viability and 96% accuracy for location) when using all 11 markers. We explored random forest models that limited the number of markers, but as features were removed the accuracy of prediction declined.

The three markers that predicted viability (PSG3, CG-Alpha, PAPPA) all affect trophoblast function and cell regulation, likely from different mechanisms. CG-Alpha, secreted from the pituitary gland may affect trophoblast growth. In contrast, PSG3 is secreted by the trophoblast and may reflect function. While PAPPA is also associated with trophoblast function, it is also associated with collagen breakdown (metalloproteinase) and cell structure. It was noted that some models predicted similarly when two biomarkers that are highly correlated were substituted (e.g. hCG and CG-Alpha, or PSG3 and PG9). Our final model included those with the best prediction and did not include hCG.

The alpha subunit of human chorionic gonadotropin (CG-Alpha) has also been examined as a potential biomarker for ectopic pregnancy. It has been theorized that ectopic pregnancies may have an impaired ability to synthesize the beta subunit, but not the alpha subunit of hCG. This may result in a lack of utilization of CG-Alpha, leading to increased levels while overall intact hCG is low (17).

PSG3 was found to predict both viability and location. Prediction was improved when used in combination with sFLT and TFPI2. sFLT is associated with abnormal angiogenesis and TFPI2 is associated with the structural constitution of the extracellular matrix. These functions may be more important in the prediction of an ectopic gestation. Of note the only two markers that were noted to be elevated in an ectopic pregnancy in our preliminary data were not selected in our final models (EHD3 and OPN).

Biomarkers must have a reliable, commercially-available assay, be validated in an external population, and achieve sufficient test characteristics before they can enter the clinical space. The development of biomarkers as a companion diagnostic is a complex process with distinct phases of identification and development (8, 18). These data represent the culmination of the first three phases of biomarker development. In the first phase, preclinical exploration identifies promising candidates (11,12). Phase two validates markers using a clinical assay in a similar population (13, 18). These data, from the third stage, demonstrate promising predictive ability in a case control study. This multiplexed biomarker panel is currently being assessed in a prospective setting (phase four of development) to validate test characteristics and determine the optimal population of use (8, 19).

While these models are not ready for immediate clinical use, these data summarized here will lead to Important next steps. Future goals include extending possible indications for individuals when transvaginal ultrasound is not expected to visualize a normal gestation, or during the evaluation of an individual with a pregnancy of unknown location. Additionally, determining the natural history and physiological role of novel biomarkers will have scientific value. It is possible that some of these markers can be used serially, similar to the use of hCG, to optimize management of individuals at risk for early pregnancy loss. Multiplexing the assays will also simplify the test and reduce potential cost.

The major limitation of an assessment of the utility of biomarkers is false positive findings (20). The chances of false positives are minimized because we conducted several investigations to validate the potential of putative and novel makers before combining them using machine leaning. We have also validated our model to optimize accuracy in independent populations (13). All specimens were carefully phenotyped based on international standards (15) and were obtained, stored, and analyzed using state of the art procedures. In the present phase of biomarker development, we have purposely compared the prediction of our biomarker test to the gold standard of diagnosis, and clinical care was not informed by our test results. Individuals who did not have a clear diagnosis or were treated presumptively as an ectopic pregnancy or miscarriage were excluded from this study.

Models based on machine learning can be data driven resulting in false positive findings. We have limited that possibility with robust internal validation. We are confident results are robust given similar findings with multiple machine learning platforms and improved prediction compared to our previous studies (13).

We have demonstrated that a small pool of markers, used in combination, can be used to classify individuals with potential early pregnancy loss as not viable and extra uterine. The overall test characteristics are modestly dependent on the number of markers used. The biomarkers CG-Alpha, PAPPA and PSGE and be used to predict viability and sFLT, TPFI2 and PSG3 can be used to predict pregnancy location.

### REFERENCES

1. Barnhart K T, Simhan H, Kamelle S A. Diagnostic accuracy of ultrasound above and below the beta-hCG discriminatory zone. Obstet Gynecol 1999; 94:583-7. doi: 10.1016/s0029-7844 (99) 00347-6.
2. Barnhart K T, Fay C A, Suescum M, et al. Clinical factors affecting the accuracy of ultrasonography in symptomatic first-trimester pregnancy. Obstet Gynecol 2011; 117:299-306. doi: 10.1097/AOG.0b013e3182050ed0.
3. Condous G, Okaro E, Khalid A, et al. The accuracy of transvaginal ultrasonography for the diagnosis of ectopic pregnancy prior to surgery. Human Reprod 2005; 20:1404-9. doi: 10.1093/humrep/deh770.
4. Morse C B, Sammel M D, Shaunik A, et al. Performance of human chorionic gonadotropin curves in women at risk for ectopic pregnancy: exceptions to the rules. Fertil Steril 2012; 97:101-6.e2. doi: 10.1016/j.fertnstert.2011.10.037.
5. Committee on Practice Bulletins-Gynecology. ACOG Practice Bulletin No. 191: Tubal Ectopic Pregnancy. Obstet Gynecol 2018; 131: e65-e77. doi: 10.1097/AOG.0000000000002464.
6. NICE Guideline 126. Ectopic pregnancy and miscarriage: diagnosis and initial management. Updated Nov. 24, 2021. Accessed Jul. 31, 2023. https://www.nice.org.uk/guidance/ng126/chapter/Recommendations.
7. BMJ Best Practice: Ectopic Pregnancy. Updated Jun. 20, 2023. Accessed Jul. 31, 2023. bestpractice.bmj.com/topics/en-us/174.
8. Pepe M S, Etzioni R, Feng A Z, et al. Phases of biomarker development for early detection of cancer. J Nat Cancer Inst 2001; 93:1054-61. doi: 10.1093/jnci/93.14.1054.
9. Rausch M E, Sammel M D, Takacs P, Chung K, Shaunik A, Barnhart K T. Development of a multiple marker test for ectopic pregnancy. Obstet Gynecol 2011; 117:573-82. doi: 10.1097/AOG.0b013e31820b3c61.
10. Senapati S, Sammel M D, Butts S F, Takacs P, Chung K, Barnhart K T. Predicting first trimester pregnancy outcome: derivation of a multiple marker test. Fertil Steril 2016; 106:1725-32. doi: 10.1016/j.fertnstert.2016.08.044.
11. Beer L A, Tang, H Y, Sriswadi S, Barnhart K T, Speicher D W. Systematic discovery of ectopic pregnancy serum biomarkers using 3-D protein profiling coupled with label-free quantitation. J Proteome Res 2011; 10:1126-38. doi: 10.1021/pr1008866.
12. Lee I T, Senapati S, Schreiber C, Koelper N, Takacs P, Barnhart K. Application of a Multiplex Platform to Identify Novel Biomarkers for Pregnancy Location and Viability. 2023 May. doi: 10.21203/rs.3.rs-2777020/v1.
13. Bollig K J, Senapati S, Sammel M D, et al. Validation of a multiple marker test for early pregnancy outcome prediction. J Assist Reprod Genet 2023; 40:837-844. doi: 10.1007/s10815-023-02719-w.
14. Barnhart K, van Mello N M, Bourne T, et al. Pregnancy of unknown location: a consensus statement of nomenclature, definitions, and outcome. Fertil Steril 2011; 95:857-66. doi: 10.1016/j.fertnstert.2010.09.006.
15. Seeber B, Sammel M D, Fan X, et al. Panel of markers can accurately predict endometriosis in a subset of patients. Fertil Steril 2008; 89:1973-81. doi: 10.1016/j.fertnstert.2007.05.014.
16. Sackett D L, Haynes R B, Guyatt G H, Tugwell P. The selection of diagnostic tests. In: Sackett D, ed. Clinical epidemiology. Boston, Toronto, and London: Little, Brown and Company; 1991:47-57.
17. Wolf G C, Medford E E, Wynn R M. Trophoblast production of hCG and ahCG from normal and abnormal pregnancies. Gynecol Obstet Invest 1994; 37:236-41. doi: 10.1159/000292568.
18. Pepe M S, Feng Z, Janes H, Bossuyt P M, Potter J D. Pivotal evaluation of the accuracy of a biomarker used for classification or prediction: standards for study design. J Natl Cancer Inst 2008; 100:1432-8. doi: 10.1093/jnci/djn326.
19. Bossuyt P M, Reitsma J B, Bruns D E, et al. Towards complete and accurate reporting of studies of diagnostic accuracy: The STARD Initiative. Ann Intern Med 2003; 138:40-4. doi: 10.7326/0003-4819-138-1-200301070-00010.
20. Palmer S S, Barnhart K T. Biomarkers in reproductive medicine: the promise, and can it be fulfilled? Fertil Steril 2013; 99:954-62. doi: 10.1016/j.fertnstert.2012.11.019.
21. Mueller M D, Raio L, Spoerri S, Ghezzi F, Dreher E, Bersinger N A. Novel placental and nonplacental serum markers in ectopic versus normal intrauterine pregnancy. Fertil Steril 2004; 81:1106-11. doi: 10.1016/j.fertnstert.2003.08.049.
22. Horne C H, Towler C M, Pugh-Humphreys R G, Thomson A W, Bohn H. Pregnancy specific beta1-glycoprotein—a product of the syncytiotrophoblast. Experientia 1976; 32:1197. doi: 10.1007/BF01927624.
23. Earl U, Wells M, Bulmer J N. Immunohistochemical characterisation of trophoblast antigens and secretory products in ectopic tubal pregnancy. Int J Gynecol Pathol 1986; 5:132-42.
24. Tornehave D, Chemnitz J, Westergaard J G, et al. Placental proteins in peripheral blood and tissues of ectopic pregnancies. Gynecol Obstet Invest 1987; 23:97-102. doi: 10.1159/000298842.
25. Witt B R, Wolf G C, Wainwright C J, Johnston P D, Thorneycroft I H. Relaxin, CA-125, progesterone, estradiol, Schwangerschaft protein, and human chorionic gonadotropin as predictors of outcome in threatened and nonthreatened pregnancies. Fertil Steril 1990; 53:1029-36.
26. Moore T, Dveksler G S. Pregnancy-specific glycoproteins: complex gene families regulating maternal-fetal interactions. Int J Dev Biol 2014; 58:273-80. doi: 10.1387/ijdb.130329gd.
27. Daponte A, Pournaras S, Polyzos N P, et al. Soluble FMS-like tyrosine kinase-1 (sFlt-1) and serum placental growth factor (PIGF) as biomarkers for ectopic pregnancy and missed abortion. J Clin Endocrinol Metab. 2011; 96: E1444-51. doi: 10.1210/jc.2011-0037.
28. Martínez-Ruiz A, Sarabia-Meseguer M D, Pérez-Fornieles J, Vílchez J A, Tovar-Zapata I, Noguera-Velasco J A. Placental growth factor, soluble fms-like tyrosine kinase 1 and progesterone as diagnostic biomarkers for ectopic pregnancy and missed abortion. Clin Biochem 2014; 47:844-7. doi: 10.1016/j.clinbiochem.2014.03.013.
29. Barnhart K, Sammel M D, Chung K, Zhou L, Hummel A C, Guo W. Decline of serum human chorionic gonadotropin and spontaneous complete abortion: defining the normal curve. Obstet Gynecol 2004; 104:975-81. doi: 10.1097/01.AOG.0000142712.80407.fd.
30. Condous G, Okaro E, Khalid A, et al. The use of a new logistic regression model for predicting the outcome of pregnancies of unknown location. Hum Reprod 2004; 19:1900-10. doi: 10.1093/humrep/deh341.
31. Condous G, Van Calster B, Kirk E, et al. Prediction of ectopic pregnancy in women with a pregnancy of unknown location. Ultrasound Obstet Gynecol 2007; 29:680-7. doi: 10.1002/uog.4015.
32. Barnhart K T, Sammel M D, Appleby D, et al. Does a prediction model for pregnancy of unknown location developed in the U K validate on a US population? Hum Reprod 2010; 25:2434-40. doi: 10.1093/humrep/deq217.
33. Horne A W, Brown J K, Tong S, Kaitu'u-Lino T. Evaluation of ADAM-12 as a Diagnostic Biomarker of Ectopic Pregnancy in Women with a Pregnancy of Unknown Location. PLOS One 2012; 7: e41442. doi: 10.1371/journal.pone.0041442.
34. Daponte A, Deligeoroglou E, Pournaras S, et al. Angiopoietin-1 and angiopoietin-2 as serum biomarkers for ectopic pregnancy and missed abortion: a case-control study. Clin Chim Acta 2013; 415:145-51. doi: 10.1016/j.cca.2012.10.031.
35. Florio P, Severi F M, Bocchi C, et al. Single serum activin a testing to predict ectopic pregnancy. J Clin Endocrinol Metab 2007; 92:1748-53. doi: 10.1210/jc.2006-2188.
36. Warrick J, Gronowski A, Moffett C, Zhao Q, Bishop E, Woodworth A. Serum activin A does not predict ectopic pregnancy as a single measurement test, alone or as part of a multi-marker panel including progesterone and hCG. Clin Chim Acta 2012; 413:707-11. doi: 10.1016/j.cca.2011.12.018.
37. Kirk E, Papageorghiou A T, Van Calster B, et al. The use of serum inhibin A and activin A levels in predicting the outcome of ‘pregnancies of unknown location’ Hum Reprod 2009; 24:2451-6. doi: 10.1093/humrep/dep066.
38. Horne A W, Shaw J L, Murdoch A, et al. Placental growth factor: a promising diagnostic biomarker for tubal ectopic pregnancy. J Clin Endocrinol Metab 2011; 96: E104-8. doi: 10.1210/jc.2010-1403.
39. Bischof P, Reyes H, Herrmann W L, Sizonenko P C. Circulating levels of pregnancy-associated plasma protein-A (PAPP-A) and human chorionic gonadotrophin (hCG) in intrauterine and extrauterine pregnancies. Br J Obstet Gynaecol 1983; 90:323-5. doi: 10.1111/j.1471-0528.1983.tb08917.x.
40. Dumps P, Meisser A, Pons D, et al. Accuracy of single measurements of pregnancy-associated plasma protein-A, human chorionic gonadotropin and progesterone in the diagnosis of early pregnancy failure. Eur J Obstet Gynecol Reprod Biol 2002; 100:174-80. doi: 10.1016/s0301-2115 (01) 00470-5.
41. Ugurlu E N, Ozaksit G, Karaer A, Zulfikaroglu E, Atalay A, Ugur M. The value of vascular endothelial growth factor, pregnancy-associated plasma protein-A, and progesterone for early differentiation of ectopic pregnancies, normal intrauterine pregnancies, and spontaneous miscarriages. Fertil Steril 2009; 91:1657-61. doi: 10.1016/j.fertnstert.2008.02.002.
42. Daponte A, Pournaras S, Zintzaras E, et al. The value of a single combined measurement of VEGF, glycodelin, progesterone, PAPP-A, HPL and LIF for differentiating between ectopic and abnormal intrauterine pregnancy. Hum Reprod 2005; 20:3163-6. doi: 10.1093/humrep/dei218.
43. Mol B W, Lijmer J G, Ankum W M, van der Veen F, Bossuyt P M. The accuracy of single serum progesterone measurement in the diagnosis of ectopic pregnancy: a meta-analysis. Hum Reprod 1998; 13:3220-7. doi: 10.1093/humrep/13.11.3220.
44. Zakizadeh F, Mahmoudzadeh-Sagheb H, Asemi-Rad A, et al. Upregulation of elafin expression in the fallopian tube of ectopic tubal pregnancies compared to the normal tubes. J Reprod Immunol 2020; 141:103136. doi: 10.1016/j.jri.2020.103136.
45. Ness R B, Mclaughlin M T, Heine R P, Bass D C, Mortimer L. Fetal fibronectin as a marker to discriminate between ectopic and intrauterine pregnancies. Am J Obstet Gynecol 1998; 179 (3 Pt 1): 697-702. doi: 10.1016/s0002-9378 (98) 70067-5.
46. Sullivan-Pyke C, Haisenleder D J, Senapati S, et al. Kisspeptin as a new serum biomarker to discriminate miscarriage from viable intrauterine pregnancy. Fertil Steril 2018; 109:137-141.e2. doi: 10.1016/j.fertnstert.2017.09.029.
47. Kyriacou C, Abbara A, Bobdiwala S, et al. VP59.15: Circulating kisspeptin levels in ectopic pregnancy and pregnancy of unknown location. Ultrasound Obstet Gynecol 2020; 56:318. https://doi.org/10.1002/uog 23299.
48. Bilban M, Ghaffari-Tabrizi N, Hintermann E, et al. Kisspeptin-10, a KiSS-1/metastin-derived decapeptide, is a physiological invasion inhibitor of primary human trophoblasts. J Cell Sci 2004; 117:1319-28. doi: 10.1242/jcs.00971.
49. Le Naour F, Rubinstein E, Jasmin C, Prenant M, Boucheix C. Severely reduced female fertility in CD9-deficient mice. Science 2000; 287:319-21. doi: 10.1126/science.287.5451.319.
50. Higginbottom A, Takahashi Y, Bolling L, et al. Structural requirements for the inhibitory action of the CD9 large extracellular domain in sperm/oocyte binding and fusion. Biochem Biophys Res Commun 2003; 311:208-14. doi: 10.1016/j.bbrc.2003.09.196.
51. Larsson A, Palm M, Basu S, Axelsson O. Insulin-like growth factor binding protein-1 (IGFBP-1) during normal pregnancy. Gynecol Endocrinol 2013; 29:129-32. doi: 10.3109/09513590.2012.730574.
52. Robinson J F, Kapidzic M, Gormley M, et al. Transcriptional dynamics of cultured human villous cytotrophoblasts. Endocrinology 2017; 158:1581-94. doi: 10.1210/en.2016-1635.

1. A diagnostic reagent or kit for use in diagnosing an ectopic pregnancy or a non-viable pregnancy in a mammalian subject comprising:
(a) a ligand that that binds to a peptide or protein selected from the biomarkers identified in Tables 2A and 2B, or
(b) a combination of ligand (a), wherein each ligand binds to a different peptide or protein;
wherein at least one of the ligands is associated with a detectable label or with a substrate.

2. The reagent or kit according to claim 1, wherein the peptide or proteins are selected from the group consisting of:
i. disintegrin and metalloproteinase domain-containing protein 12 (ADAM12),
ii. Glycoprotein hormones alpha chain (CGA),
iii. beta human chorionic gonadotropin (hCG),
iv. Pappalysin-1 (PAPPA),
v. pregnancy-specific beta-1-glycoprotein 1 (PSG1),
vi. pregnancy-specific beta-1-1glycoprotein 3 (PSG3),
vii. pregnancy-specific beta-1-glycoprotein 9 (PSG9),
viii. soluble fms-like tyrosine kinase-1 (sFLT),
ix. growth-differentiation faction-15 (GDF15), and
x. progesterone (PRG),
xi. and tissue factor pathway inhibitor 2 (TFPI2), or
xii. a combination of ligands, each binding a peptide or protein of (i) through (xi).

3. The reagent or kit according to claim 1, wherein the kit diagnoses pregnancy viability and the peptides or proteins are one of
a) PSG3;
b) PAPPA and PSG3;
c) CGA and PAPPA;
d) CGA and PSG3;
e) CGA, PAPPA, and PSG3; or
f) PSG3, PSG9, CGA, hCG, PRG, and GF15;
g) PSG3 and PSG9;
h) ADAM12, CGA, hCG, PAPPA, PSG1, PSG3, PSG9, sFLT, GDF15, and PRG.

4.-7. (canceled)

8. The reagent or kit according to claim 1, wherein the kit diagnoses pregnancy location and the peptides or proteins are one of:
a) PSG3 and TFPI2;
b) sFLT;
c) PGS1;
d) sFLT, PSG3, and TFPI2;
e) PSG1, PSG3, and TFPI2; or
f) ADAM12, CGA, hCG, PAPPA, PSG1, PSG3, PSG9, sFLT, GDF15, PRG, and TFPI2;
g) PSG1, sFLT, GDF15, PSG9, hCG, and CGA.

9.-17. (canceled)

18. The reagent or kit according to claim 1, wherein the peptides or proteins are all of biomarkers (i)-(xi).

19. A diagnostic reagent or kit for use in diagnosing an ectopic pregnancy in a mammalian subject comprising:
(a) a ligand that that binds to a peptide or protein selected from the group consisting of:
i. pregnancy specific beta-1-glycoprotein 9 (PSG9),
ii. pregnancy specific beta-1 glycoprotein 1 (PSG1),
iii. insulin-like growth factor binding protein 1 (IGFBP1),
iv. kisspeptin (KISS1),
v. pregnancy specific beta-1 glycoprotein 3 (PSG3), and
vi. beta-parvin (PARVB); or
(b) a combination of ligand (a), wherein each ligand binds to a different peptide or protein (i) through (vi);
wherein at least one of the ligands is associated with a detectable label or with a substrate.

20. The reagent or kit according to claim 19, further comprising:
(c) a ligand that binds to a protein or peptide fragment selected from the group consisting of:
vii. EH domain-containing protein 3 (EHD3);
viii. WAP four-disulfide core domain protein 2 (HE4); and
ix. quiescin sulfhydryl oxidase 2 (QSOX2); or
(d) a combination of ligands, each ligand binding a different peptide or protein of (vii) through (ix).

21.-26. (canceled)

27. The reagent or kit according to claim 1, which comprises a substrate upon which said polynucleotide or oligonucleotide or ligand is immobilized.

28. The reagent or kit according to claim 1, wherein said ligands or said expression products are proteins or peptides.

29. The reagent or kit according to claim 28, wherein said ligand is an antibody or fragment thereof.

30. The reagent or kit according to claim 1, wherein the polynucleotide or oligonucleotide sequence or ligand is associated with a detectable label.

31. The reagent or kit according to claim 1, comprising a microarray, a microfluidics card, a chip or a chamber.

32. (canceled)

33. A method for diagnosing an ectopic pregnancy or a non-viable pregnancy in a mammalian subject comprising:
(a) measuring in a biological fluid sample of the subject the expression level of a gene, gene fragment, gene transcript or expression product selected from the biomarkers identified in Tables 2A and 2B, and
(b) comparing said subject's selected gene, gene fragment, gene transcript or expression product expression level with the level of the same gene, gene fragment, gene transcript or expression product in the biological fluid of a reference or control female mammalian subject having a normal intrauterine pregnancy (IUP),
wherein changes in expression of the subject's selected gene, gene fragment, gene transcript or expression products from those of the reference or control correlates with a diagnosis of ectopic pregnancy or non-viable pregnancy.

34. The method according to claim 33, wherein the biomarkers are selected from the group consisting of:
i. disintegrin and metalloproteinase domain-containing protein 12 (ADAM12),
ii. Glycoprotein hormones alpha chain (CGA),
iii. beta human chorionic gonadotropin (hCG),
iv. Pappalysin-1 (PAPPA),
v. pregnancy-specific beta-1-glycoprotein 1 (PSG1),
vi. pregnancy-specific beta-1-1glycoprotein 3 (PSG3),
vii. pregnancy-specific beta-1-glycoprotein 9 (PSG9),
viii. soluble fms-like tyrosine kinase-1 (sFLT),
ix. growth-differentiation faction-15 (GDF15), and
x. progesterone (PRG), and tissue factor pathway inhibitor 2 (TFPI2), or
xi. a combination of ligands, each binding a peptide or protein of (i) through (x).

35. The method according to claim 33, wherein the method diagnoses pregnancy viability and the peptides or proteins are one of:
a) PSG3;
b) PAPPA and PSG3;
c) CGA and PAPPA;
d) CGA and PSG3;
e) CGA, PAPPA, and PSG3; or
f) PSG3, PSG9, CGA, hCG, PRG, and GF15;
g) PSG3 and PSG9;
h) ADAM12, CGA, hCG, PAPPA, PSG1, PSG3, PSG9, sFLT, GDF15, and PRG.

36.-39. (canceled)

40. The method according to claim 33, wherein the method diagnoses pregnancy location and the peptides or proteins are one of:
a) PSG3 and TFPI2;
b) sFLT;
c) PGS1;
d) sFLT, PSG3, and TFPI2;
e) PSG1, PSG3, and TFPI2; or
f) ADAM12, CGA, hCG, PAPPA, PSG1, PSG3, PSG9, sFLT, GDF15, PRG, and TFPI2;
g) PSG1, sFLT, GDF15, PSG9, hCG, and CGA.

41.-49. (canceled)

50. The method according to claim 33, wherein the peptides or proteins are all of biomarkers (i)-(xi).

51. (canceled)

62. The method of claim 33, further comprising terminating the non-viable or ectopic pregnancy.

63.-80. (canceled)
